(19) United States
(12) Patent Application Publication
(10) Pub. No.: US 2023/0416809 A1
(43) Pub. Date: Dec. 28, 2023

(54) SPATIAL DETECTION OF BIOMOLECULE INTERACTIONS

(71) Applicant: Singular Genomics Systems, Inc., San Diego, CA (US)

(72) Inventor: Christian BERRIOS, San Diego, CA (US)

(21) Appl. No.: 18/339,628

(22) Filed: Jun. 22, 2023

Related U.S. Application Data
(60) Provisional application No. 63/354,846, filed on Jun. 23, 2022.
(60) Provisional application No. 63/497,554, filed on Apr. 21, 2023.

(51) Int. Cl. C12Q 1/6841

(52) U.S. Cl. CPC C12Q 1/6841

(57) ABSTRACT

Disclosed herein, inter alia, are compositions and methods for spatial detection of biomolecular interactions.

### CROSS-REFERENCES TO RELATED APPLICATIONS

This application claims the benefit of U.S. Provisional Application No. 63/497,554, filed Apr. 21, 2023, which claims the benefit of U.S. Provisional Application No. 63/354,846, filed Jun. 23, 2022, which are incorporated herein by reference in their entirety and for all purposes.

### SEQUENCE LISTING

The Sequence Listing written in file 051385-582001US_ST.26_SEQUENCE_LISTING.xml, created on Jun. 15, 2023, 73,991 bytes, machine format IBM-PC, MS Windows operating system, is hereby incorporated by reference.

### BACKGROUND

The study of proteins is emerging as a new frontier for understanding real-time human biology. Protein biomarker discovery enables identification of signatures with pathophysiological importance, bridging the gap between genomes and phenotypes. This type of data will have a profound impact on improving future healthcare, particularly with respect to precision medicine, but progress has been hampered by the lack of technologies that can provide reliable specificity, high throughput, good precision, and high sensitivity. Expanding the knowledge of cellular protein interaction networks is vital to improve our understanding of several types of diseases, including cancer. Improved methods to study these interaction networks, especially in clinical settings, is therefore of great importance both for increasing the knowledge of the underlying disease mechanics, and also for finding new biomarkers for improved disease diagnostics and treatment response prediction. Disclosed herein, inter alia, are solutions to these and other problems in the art.

### BRIEF SUMMARY

In an aspect is provided a method of forming an oligonucleotide including two barcode sequences, the method including: a) contacting a first biomolecule with a first proximity probe, wherein the first proximity probe includes a first oligonucleotide including, from 5′ to 3′, a first primer binding sequence, a first barcode sequence, and a first probe sequence; b) contacting a second biomolecule with a second proximity probe, wherein the second proximity probe includes a second oligonucleotide including, from 5′ to 3′, a second primer binding sequence, a second barcode sequence, and a second probe sequence; c) hybridizing the first probe sequence of the first oligonucleotide to the second probe sequence of the second oligonucleotide and extending the first probe sequence with a polymerase to form a first extended oligonucleotide including, from 5′ to 3′, the first primer binding sequence, the first barcode sequence, the first probe sequence, a complement of the second barcode sequence, and a complement of the second primer binding sequence.

In an aspect is provided a composition including: i) a biomolecule bound to a proximity probe, wherein the proximity probe includes an extended probe oligonucleotide including, from 5′ to 3′, a first primer binding sequence, a first barcode sequence, a first probe sequence, a complement of a second barcode sequence, and a complement of a second primer binding sequence; and ii) an oligonucleotide primer hybridized to the extended probe oligonucleotide, wherein the oligonucleotide primer includes, from 5′ to 3′, a first sequence complementary to the first primer binding sequence and a second sequence complementary to the complement of the second primer binding sequence.

In an aspect is provided a composition including: i) a biomolecule bound by a proximity probe, wherein the proximity probe includes an extended probe oligonucleotide including, from 5′ to 3′, a first primer binding sequence, a first barcode sequence, a first probe sequence, a complement of a second barcode sequence, a complement of a third probe sequence, a complement of a third barcode sequence, a complement of a fifth probe sequence, an internal cleavable site, and a complement of a second primer binding sequence; and ii) an oligonucleotide primer hybridized to the extended probe oligonucleotide, wherein the oligonucleotide primer includes, from 5′ to 3′, a first sequence complementary to the first primer binding sequence and a second sequence complementary to the complement of the second primer binding sequence.

### BRIEF DESCRIPTION OF THE DRAWINGS

FIGS. 1A-1E illustrate embodiments of proximity probes (e.g., oligonucleotide-conjugated antibodies). FIG. 1A shows an embodiment of an oligonucleotide-conjugated proximity probe, referred to herein as a first proximity probe (or also referred to as a primary proximity probe). The first proximity probe includes a specific binding molecule (e.g., an antibody, affimer, aptamer, etc.) linked to a first probe oligonucleotide (also referred to herein as a first oligonucleotide or a primary probe oligonucleotide). The first probe oligonucleotide includes, from 5′ to 3′, a first primer binding sequence (PB1; also referred to herein as a first padlock probe (PLP) binding sequence), a first barcode sequence (UMI1; also referred to herein as a first unique molecular identifier), and a first probe sequence (PS1; also referred to herein as a first oligo interaction sequence). FIG. 1B shows an embodiment of a second proximity probe (or also referred to as a secondary proximity probe). The secondary proximity probe includes a specific binding molecule (e.g., an antibody, affimer, aptamer, etc.) linked to a second probe oligonucleotide (also referred to herein as a second oligonucleotide or a secondary probe oligonucleotide). The second probe oligonucleotide includes, from 5′ to 3′, a cleavable site, a second primer binding sequence (PB2; also referred to herein as a second padlock probe (PLP) binding sequence), a second barcode sequence (UMI2; also referred to herein as a second unique molecular identifier), and a complement to the first probe sequence (PS1′). FIG. 1C illustrates an alternate embodiment of a second proximity probe that includes two orthogonal cleavable sites. The second probe oligonucleotide includes, from 5′ to 3′, a first cleavable site, a second primer binding sequence (PB2), a second internal cleavable site, a third probe sequence (PS3; also referred to herein as a third oligo interaction sequence), a second barcode sequence (UMI2), and a second probe sequence (PS2; also referred to herein as a second oligo interaction sequence). The second cleavable site (also referred to herein as a second internal cleavable site) may be cleaved by an orthogonal mechanism to the first cleavable site (e.g., the first cleavable site is cleaved by a RNAse and the second internal cleavable site is cleaved by a restriction endonuclease). FIG. 1D illustrates a circularizable probe (CP; also referred to herein as a padlock probe or gap-fill padlock probe). The circularizable probe includes, from 5′ to 3′, a first primer binding sequence complement (PB1′), optionally, one or more primer binding sequences (e.g., one or more sequencing primer binding sequences and/or one or more amplification primer binding sequences), and a second primer binding sequence (PB2), wherein, for example, the PB1′ sequence of the circularizable probe is complementary to the PB1 sequence of the first probe oligonucleotide, and the PB2 sequence of the circularizable probe is complementary to the PB2′ sequence of the second probe oligonucleotide, as described herein. FIG. 1E illustrates an embodiment of the first proximity probe described in FIG. 1A, wherein the probe sequence (PS1) is hybridized to a blocking element, thereby preventing non-specific hybridization of the probe sequence and complement of the probe sequence on the first and second probe oligonucleotides.

FIGS. 2A-2D illustrate in situ protein targeting embodiments using the proximity probes (e.g., oligonucleotide-conjugated antibodies) described herein, wherein one or more first proximity probes and second proximity probes bind to a protein complex within a cell and/or a tissue sample. FIG. 2A illustrates a protein complex in a cell including Protein A and Protein B, wherein a first proximity probe is bound to Protein A and a second proximity probe is bound to Protein B. Under suitable hybridization conditions, the PS1 sequence of the first probe oligonucleotide anneals to the PS1′ sequence of a proximal second probe oligonucleotide. FIG. 2B illustrates a protein in a cell (e.g., Protein A), wherein a first proximity probe is bound to Protein A and a second proximity probe is also bound to Protein A. Under suitable hybridization conditions, the PS1 sequence of the first probe oligonucleotide anneals to the PS1′ sequence of a proximal second probe oligonucleotide. FIG. 2C illustrates a protein complex in a cell including two copies of Protein A (e.g., a Protein A dimer), wherein an oligonucleotide-conjugated first proximity probe is bound to each copy. In this case, there will be no hybridization between the two probe oligonucleotides, as the two PS1 sequences are not complementary. FIG. 2D illustrates a protein complex including Protein A, Protein B, Protein C, and Protein D, wherein three oligonucleotide-conjugated first proximity probes are bound to Protein A (e.g., wherein each of the three proximity probes targets a different epitope on Protein A), and an oligonucleotide-conjugated second proximity probe is bound to each of Protein B, Protein C, and Protein D (e.g., wherein each second proximity probe is specific for either Protein B, Protein C, or Protein D). Under suitable hybridization conditions the PS1 sequence of each first probe oligonucleotide anneals to the PS1′ sequence of a proximal second probe oligonucleotide. In embodiments, not every first proximity probe bound to a single protein (e.g., bound to Protein A) will be proximal and associate with a second proximity probe.

FIGS. 3A-3D illustrate an embodiment of a method described herein for spatial detection of protein interactions using the proximity probes (e.g., oligonucleotide-conjugated antibodies) described herein. FIG. 3A illustrates a protein complex in a cell, wherein the complex includes Protein A bound to Protein B. A first proximity probe is bound to Protein A and is proximal to a second proximity probe bound to Protein B, such that the first and second probe oligonucleotides hybridize, as described in FIG. 2A. Using a polymerase, the 3′ end of each hybridized probe oligonucleotide is extended, generating a first extended oligonucleotide conjugated to the first proximity probe including, from 5′ to 3′, a first primer binding sequence (PB1), a first barcode sequence (UMI1), a first probe sequence (PS1), a complement of the second barcode sequence (UMI2′), and a complement of the second primer binding sequence (PB2′), and a second extended oligonucleotide conjugated to the secondary proximity antibody including, from 5′ to 3′, a second primer binding sequence (PB2), a second barcode sequence (UMI2), a complement of the first probe sequence (PS1′), a complement of the first barcode sequence (UMI1′), and a complement to the first primer binding sequence (PB1′). The cleavable site on the second probe oligonucleotide is then cleaved (e.g., RNAse cleavage of a ribonucleotide at or near the 5′ end of the second probe oligonucleotide), releasing the strand from the proximity probe (e.g., the antibody). FIG. 3B illustrates the steps of removing the cleaved strand (e.g., by lambda exonuclease 5′ to 3′ digestion), and subsequently hybridizing a circularizable probe onto the target nucleic acid sequence, wherein the PB1′ region at the 5′ end of the probe anneals to the PB1 sequence of the oligonucleotide, and wherein the PB2 region at the 3′ end anneals to the PB2′ sequence of the oligonucleotide. FIG. 3C illustrates the steps of extending the 3′ end of the circularizable probe (e.g., using a non-strand displace polymerase) to generate a complementary sequence, including from 3′ to 5′, the second barcode sequence (UMI2), the complement of the first probe sequence (PS1′), and the complement of the first barcode sequence (UMI1′). Following extension, the 3′ end of the complementary sequence is ligated to the 5′ end of the circularizable probe using, for example, a ligase, thereby generating a circularized probe. FIG. 3D illustrates the steps of amplifying the circularized probe (e.g., by rolling circle amplification using a processive strand-displacing polymerase), thereby generating a concatemer of amplification products. The amplification products are then detected, for example, by hybridizing a sequencing primer to a plurality of sequencing primer binding sequences on the amplification product, incorporated a labeled nucleotide (shown as a star) with a polymerase (shown as a cloud-like object), and detecting the label to identify the incorporated base. The amplification products may also be detected using fluorescently labeled probes. In embodiments, detection includes identifying the barcode sequence(s).

FIG. 4 illustrates a circularized probe (e.g., of FIG. 3C), primed with an amplification primer and extended with a strand-displacing polymerase to generate a concatemer containing multiple copies of the target nucleic acid sequence. The different colors in the resulting concatemer amplification product represents the generation of multiple copies of the original barcode are formed in the amplification product.

FIG. 5 is a schematic illustration of embodiments of the oligonucleotide primer (e.g., circularizable probe, such as a gap-fill padlock probe) described herein. In embodiments, the padlock probe (PLP) is a single-stranded oligonucleotide containing a first complementary region and a second complementary region (i.e., nucleic acid sequences complementary to nucleic acid sequences flanking the target nucleic acid sequence). In embodiments, the padlock probe further includes an amplification priming site (i.e., a nucleic acid sequence complementary to an amplification primer) and a distinct sequencing priming site (i.e., a nucleic acid sequence complementary to a sequencing primer). Alternatively, in embodiments, the padlock probe further includes an amplification priming site and a sequencing priming site that are the same, are partially overlapping, or in which one is internal to the other. The relative size of the constituents (e.g., complementary regions and/or priming sites) as illustrated in FIG. 5 is not indicative of the overall length.

FIGS. 6A-6F illustrate an embodiment of the methods described herein for detecting protein interactions, including a protein complex in situ using the proximity probes (e.g., oligonucleotide-conjugated antibodies) described herein. FIG. 6A illustrates a protein complex in a cell including Protein A, Protein B, and Protein C. A first proximity probe (as described in FIG. 1A) is bound to Protein A, and a second proximity probe and third proximity probe (each as described in FIG. 1C, each including both a first cleavable site and a second internal cleavable site), wherein the second proximity probe is bound to Protein B and the third proximity probe is bound to Protein C. Under conditions suitable for hybridization of the probe oligonucleotides (e.g., a buffered solution of suitable ionic strength for nucleic acid hybridization), two different probe oligonucleotide duplexes are possible between the first proximity probe bound to Protein A and either the second proximity probe bound to Protein B or the third proximity probe bound to Protein C. To aid the eye in orientating the probe oligonucleotides through each of the following figures, the probe sequences of each probe oligonucleotide have been labeled with a number (e.g., 1, 2, 3, 4 or 5), although it is to be understood that this does not imply that each of the probe sequences are necessarily different from one another (e.g., in some instances, two probe sequences may include the same sequence, such as the probe sequences of the second and third proximity probes). FIG. 6B illustrates extension of the annealed Protein A and Protein C probe oligonucleotides, wherein the first probe sequence (1) of the first probe oligonucleotide is duplexed to the second probe sequence (2) of the second probe oligonucleotide. Using a polymerase, the 3′ end of each hybridized probe oligonucleotide is extended, generating: a first extended oligonucleotide conjugated to the first proximity probe including, from 5′ to 3′, a first primer binding sequence (PB1), a first barcode sequence (UMI1), the first probe sequence (1), a complement to the second barcode sequence (UMI2′), a complement to the third probe sequence (2′), a cleavable complement of the second internal cleavable site, and a complement to the second primer binding sequence (PB2′); and a second extended oligonucleotide conjugated to the second proximity probe including, from 5′ to 3′, a second primer binding sequence (PB2), a second internal cleavable site, a third probe sequence (3), a second barcode sequence (UMI2), a second probe sequence (2, a complement of the first barcode sequence (UMI1′), and a complement of first primer binding sequence complement (PB1′). The second internal cleavable site of the second probe oligonucleotide and the cleavable complement of the second internal cleavable site are then cleaved (e.g., by endonuclease digestion with an enzyme that recognizes the duplexed second cleavable site and cleavable complement of the second cleavable site, as illustrated by the lightning bolts), releasing the second extended oligonucleotide from the second proximity probe. FIG. 6C illustrates the steps of removing the cleaved second probe oligonucleotide (e.g., by lambda exonuclease digestion at the free 5′-PO₄ of the second probe oligonucleotide), and subsequently hybridizing the first probe oligonucleotide to the third probe oligonucleotide on Protein B, wherein the complement of the third probe sequence (3′) of the first probe oligonucleotide anneals to the fourth probe sequence (4) of the third probe oligonucleotide. FIG. 6D illustrates extension of the annealed Protein A and Protein B probe oligonucleotides. Using a polymerase, the 3′ end of each hybridized probe oligonucleotide is extended, generating: a third extended oligonucleotide including, from 5′ to 3′, the first primer binding sequence (PB1), the first barcode sequence (UMI1), the first probe sequence (1), the complement of the second barcode sequence (UMI2′), the complement of the third probe sequence (3′), a complement of the third barcode sequence (UMI3′), a complement of the fifth probe sequence (5′), a complement of the second internal cleavable site, and the complement of the second primer binding sequence (PB2′); and a fourth extended oligonucleotide including, from 5′ to 3′, a second PLP binding sequence (PB2), a second internal cleavable site, a fifth probe sequence (5), a third barcode sequence (UMI3), the fourth probe sequence (4), the second barcode sequence (UMI2), the complement of the first probe sequence (1′), a complement of the first barcode sequence (UMI1′), and a complement of the first primer binding sequence (PB1′). The first cleavable site on the fourth extended oligonucleotide is then cleaved (e.g., RNAse cleavage of a ribonucleotide), releasing the fourth extended oligonucleotide from the antibody. FIG. 6E illustrates the steps of removing the cleaved fourth extended oligonucleotide (e.g., by lambda exonuclease 5′ to 3′ digestion), and subsequently hybridizing a circularizable probe onto the third extended oligonucleotide, wherein the PB1′ region at the 5′ end of the probe anneals to the PB1 sequence of the third extended oligonucleotide, and wherein the PB2 region at the 3′ end anneals to the PB2′ sequence of the third extended oligonucleotide. FIG. 6F illustrates the steps of extending the 3′ end of the circularizable probe (e.g., using a non-strand displacing polymerase) to generate a complementary sequence, including from 3′ to 5′, the second internal cleavable site, a fifth probe sequence (5), a third barcode sequence (UMI3), the third probe sequence (3), the second barcode sequence (UMI2), the complement of the first barcode sequence (1′), and the complement of the first barcode sequence (UMI1′). Following extension, the 3′ end of the complementary sequence is ligated to the 5′ end of the circularizable probe using, for example, a ligase, thereby generating a circularized probe. The circularized probe may then be amplified and detected, for example by sequencing, as described in FIG. 3D.

### DETAILED DESCRIPTION

The aspects and embodiments described herein relate to compositions and method for spatial detection of biomolecules.

### I. Definitions

All patents, patent applications, articles and publications mentioned herein, both supra and infra, are hereby expressly incorporated herein by reference in their entireties.

The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices, and materials similar or equivalent to those described herein can be used in the practice of embodiments of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the K_{D} (equilibrium dissociation constant) between two specific binding molecules is less than 10⁻⁶M, less than 10⁻⁷M, less than 10⁻⁸ M, less than 10⁻⁹M, less than 10⁻⁹M, less than 10⁻¹¹ M, or less than about 10⁻¹² M or less.

As used herein, the term “contacting” is used in accordance with its plain ordinary meaning and refers to the process of allowing at least two distinct species (e.g., chemical compounds, biomolecules, nucleotides, binding reagents, or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, a protein (e.g., an antibody), or enzyme.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

As used herein, the term “proximity probe” is used in accordance with its plain ordinary meaning and refers to a specific binding agent (e.g., an antibody) attached to an oligonucleotide. In embodiments, pairs or sets of proximity probes can be employed to target multiple biomolecules of interest. Alternatively, in embodiments, a pair of proximity probes may be employed for a single biomolecule of interest. When different proximity probes harboring complementary oligonucleotides are adjacent, these oligonucleotides can be ligated, extended, and/or amplified to facilitate the detection of proteins and/or complexes. Examples of biological assay that utilize proximity probes include proximity ligation assay (PLA) and proximity extension assay (PEA). In addition, proximity probes include an antibody fragment, an affimer, an aptamer, or nucleic acid to facilitate interaction between biomolecule of interest.

As used herein, the term “affimer” is used in accordance with its plain ordinary meaning and refers to non-antibody binding proteins. These small proteins bind to target proteins with nanomolar affinity to facilitate the labelling of biomolecules in cells. An example of affimer includes, and is not limited to, Affimer® Technology, which is commercialized by Avacta® for diagnostic applications.

As used herein, the term “aptamer” is used in accordance with its plain ordinary meaning and refers to oligonucleotide or peptide molecules that bind to a specific target molecule. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In embodiments, peptide aptamers consist of one (or more) short variable peptide domains, attached at both ends to a protein scaffold. Aptamers may be designed with any combination of the base modified nucleotides desired. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target with a much higher degree of affinity than it binds to other, non-target, components in a mixture or sample. An aptamer can be identified using any known method, including the SELEX process. See, e.g., U.S. Pat. No. 5,475,096 entitled “Nucleic Acid Ligands”. Once identified, an aptamer can be prepared or synthesized in accordance with any known method, including chemical synthetic methods and enzymatic synthetic methods.

Nucleic acid aptamers are nucleic acid species that are typically the product of engineering through repeated rounds of in vitro selection, such as SELEX (systematic evolution of ligands by exponential enrichment), to bind to various molecular targets such as small molecules, proteins, nucleic acids, and even cells, tissues and organisms. At the molecular level, aptamers bind to its target site through non-covalent interactions. Aptamers bind to these specific targets because of electrostatic interactions, hydrophobic interactions, and their +complementary shapes. In embodiments, peptide aptamers are artificial proteins selected or engineered to bind specific target molecules. These proteins may include or consist of one or more peptide loops of variable sequence displayed by a protein scaffold. They are typically isolated from combinatorial libraries and often subsequently improved by directed mutation or rounds of variable region mutagenesis and selection. An example of an aptamer is Macugen, which is a pegylated aptamer that targets the growth factor, VEFG165. (See Ni et al. ACS Appl Mater Interfaces. 2021 Mar. 3; 13(8):9500-9519 and Song et al. Sensors (Basel). 2012; 12(1):612-31).

As used herein, the term “complement,” refers to a nucleotide (e.g., RNA or DNA) or a sequence of nucleotides capable of base pairing with a complementary nucleotide or sequence of nucleotides. For example, complementarity exists between the two strands of a double stranded DNA molecule or between an oligonucleotide primer and a primer binding site on a single stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence, only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often comprise nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, one or both sequences in a pair of complementary sequences form portions of longer polynucleotides, which may or may not include additional regions of complementarity.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that are to ligated together will generally directly abut one another.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “polynucleotide template” refers to any polynucleotide molecule that may be bound by a polymerase and utilized as a template for nucleic acid synthesis. As used herein, the term “polynucleotide primer” refers to any polynucleotide molecule that may hybridize to a polynucleotide template, be bound by a polymerase, and be extended in a template-directed process for nucleic acid synthesis, such as in a PCR or sequencing reaction. Polynucleotide primers attached to a core polymer within a core are referred to as “core polynucleotide primers.” A primer can be of any length depending on the particular technique it will be used for. For example, amplification primers are generally between 10 and 40 nucleotides in length. The length and complexity of the nucleic acid fixed onto the nucleic acid template may vary. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide.

As used herein, the term “template polynucleotide” refers to any polynucleotide molecule that may be bound by a polymerase and utilized as a template for nucleic acid synthesis. In general, the terms “target polynucleotide” and “target nucleic acid” are used interchangeably herein refer to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

The term “adapter” as used herein refers to any oligonucleotide that can be ligated to a nucleic acid molecule, thereby generating nucleic acid products that can be sequenced on a sequencing platform (e.g., an Illumina or Singular Genomics™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., primer sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In embodiments, the adapter can include an index sequence (also referred to as barcode or tag) to assist with downstream error correction, identification or sequencing. In some embodiments, an adapter is hairpin adapter. In some embodiments, a hairpin adapter comprises a single nucleic acid strand comprising a stem-loop structure. In some embodiments, a hairpin adapter comprises a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter comprises a stem portion (i.e., stem) and a loop, wherein the stem portion is substantially double stranded thereby forming a duplex. In some embodiments, the loop of a hairpin adapter comprises a nucleic acid strand that is not complementary (e.g., not substantially complementary) to itself or to any other portion of the hairpin adapter. In some embodiments, a method herein comprises ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may comprise different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may comprise different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Other analog nucleic acids include bis-locked nucleic acids (bisLNAs; e.g., including those described in Moreno PMD et al. Nucleic Acids Res. 2013; 41(5):3257-73), twisted intercalating nucleic acids (TINAs; e.g., including those described in Doluca O et al. Chembiochem. 2011; 12(15):2365-74), bridged nucleic acids (BNAs; e.g., including those described in Soler-Bistue A et al. Molecules. 2019; 24(12): 2297), 2′-O-methyl RNA:DNA chimeric nucleic acids (e.g., including those described in Wang S and Kool E T. Nucleic Acids Res. 1995; 23(7):1157-1164), minor groove binder (MGB) nucleic acids (e.g., including those described in Kutyavin IV et al. Nucleic Acids Res. 2000; 28(2):655-61), morpholino nucleic acids (e.g., including those described in Summerton J and Weller D. Antisense Nucleic Acid Drug Dev. 1997; 7(3):187-95), C5-modified pyrimidine nucleic acids (e.g., including those described in Kumar P et al. J. Org. Chem. 2014; 79(11): 5047-5061), peptide nucleic acids (PNAs; e.g., including those described in Gupta A et al. J. Biotechnol. 2017; 259: 148-59), and/or phosphorothioate nucleotides (e.g., including those described in Eckstein F. Nucleic Acid Ther. 2014; 24(6):374-87).

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H4)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H4), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules (e.g., an internal cleavable site). In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase. Cleavage agents used in methods described herein may be selected from nicking endonucleases, DNA glycosylases, or any single-stranded cleavage agents described in further detail elsewhere herein. Enzymes for cleavage of single-stranded DNA may be used for cleaving heteroduplexes in the vicinity of mismatched bases, D-loops, heteroduplexes formed between two strands of DNA which differ by a single base, an insertion or deletion. Mismatch recognition proteins that cleave one strand of the mismatched DNA in the vicinity of the mismatch site may be used as cleavage agents. Nonenzymatic cleaving may also be done through photodegredation of a linker introduced through a custom oligonucleotide used in a PCR reaction.

As used herein, the term “cleavable complement” refers to a nucleotide (e.g., RNA or DNA) or a sequence of nucleotides capable of base pairing with a complementary nucleotide or sequence of nucleotides, wherein the complementary nucleotide or sequence of nucleotides includes a cleavable site, and the cleavable complement also includes a complement to the cleavable site. In embodiments, the cleavable complement of the cleavable site and the cleavable site are cleaved by the same mechanism (e.g., restriction enzyme digestion of the duplexed cleavable site and cleavable complement of the cleavable site).

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

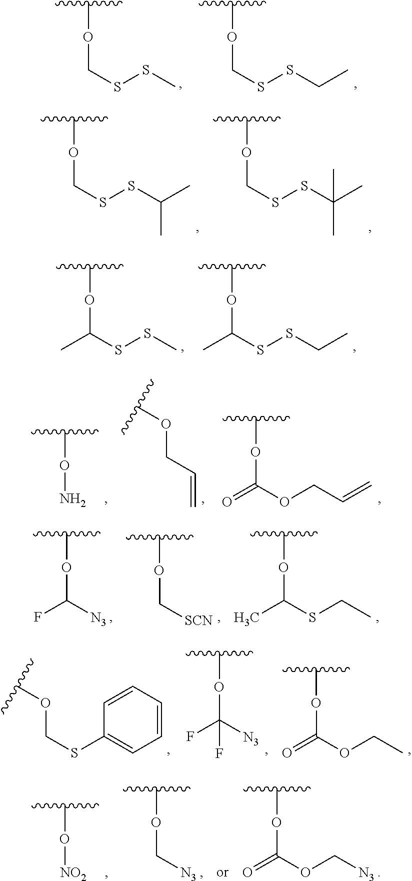

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

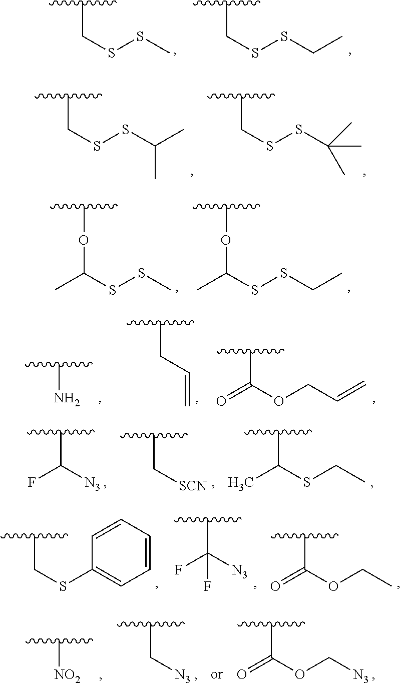

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

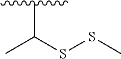 [SMILES: *C(C)SSC]

as described in U.S. Pat. No. 10,738,072, which is incorporated herein by reference for all purposes. For example, a nucleotide including a reversible terminator moiety may be represented by the formula:

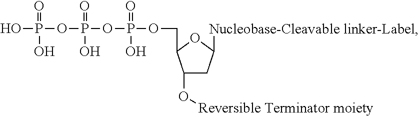

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcode sequence is a nucleic acid sequence (e.g., 8 to 24 nucleotides) from a known set of barcode sequences. In embodiments, each barcode sequence is unique within the known set of barcodes. In embodiments, the barcodes are selected to form a known set of barcodes, e.g., the set of barcodes may be distinguished by a particular Hamming distance. In embodiments, a barcode is associated with a particular proximity probe. In embodiments, a set of barcodes is associated with a particular proximity probe.

In embodiments, a nucleic acid (e.g., a probe or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at either the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996).

As used herein, the term “endonuclease” refers to enzymes that cleave the phosphodiester bond within a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” An endonuclease may cut a double-stranded polynucleotide on a single strand. The methods and compositions described herein may be applied to cleavage sites generated by endonucleases. In some alternatives of the system, the system can further provide nucleic acids that encode an endonuclease, such as Cas9, TALEN, or MegaTAL, or a fusion protein comprising a domain of an endonuclease, for example, Cas9, TALEN, or MegaTAL, or one or more portion thereof. These examples are not meant to be limiting and other endonucleases and alternatives of the system and methods comprising other endonucleases and variants and modifications of these exemplary alternatives are possible without undue experimentation. All such variations and modifications are within the scope of the current teachings.

As used herein, the term “nicking endonuclease” refers to any enzyme, naturally occurring or engineered, that is capable of breaking a phosphodiester bond on a single DNA strand, leaving a 3′-hydroxyl at a defined sequence. Nicking endonucleases can be engineered by modifying restriction enzymes to eliminate cutting activity for one DNA strand, or produced by fusing a nicking subunit to a DNA binding domain, for example, zinc fingers and DNA recognition domains from transcription activator-like effectors.

As used herein, “nick” generally refers to enzymatic cleavage of only one strand of a double-stranded nucleic acid at a particular region, while leaving the other strand intact, regardless of whether one or more bases are removed. In some cases, one or more bases are removed while in other cases no bases are removed and only phosphodiester bonds are broken. In some instances, such cleavage events leave behind intact double-stranded regions lacking nicks that are a short distance apart from each other on the double-stranded nucleic acid, for example a distance of about or at least about 1, 2, 3, 4, 5, 6, 7, 8, 9, 10, 11, 12, 13, 14, 15 bases or more. In some cases, the distance between the intact double-stranded regions is equal to or less than 15, 14, 13, 12, 11, 10, 9, 8, 7, 6, 5, 4, 3, or 2 bases. In some instances, the distance between the intact double-stranded regions is 2 to 10 bases, 3 to 9 bases, or 4 to 8 bases.

As used herein, the term “incorporating” or “chemically incorporating,” when used in reference to a primer and cognate nucleotide, refers to the process of joining the cognate nucleotide to the primer or extension product thereof by formation of a phosphodiester bond.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) are well known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “sequencing”, “sequence determination”, and “determining a nucleotide sequence”, are used in accordance with their ordinary meaning in the art, and refer to determination of partial as well as full sequence information of the nucleic acid being sequenced, and particular physical processes for generating such sequence information. That is, the term includes sequence comparisons, fingerprinting, and like levels of information about a target nucleic acid, as well as the express identification and ordering of nucleotides in a target nucleic acid. The term also includes the determination of the identification, ordering, and locations of one, two, or three of the four types of nucleotides within a target nucleic acid. Sequencing produces one or more sequencing reads.

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to binding and/or incorporating one or more nucleotides (e.g., a compound described herein) to the 3′ end of a polynucleotide with a polymerase, and detecting one or more labels that identify the one or more nucleotides. The sequencing may be accomplished by, for example, sequencing by synthesis, sequencing by binding, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic. In some embodiments, the alternating layers of polymeric gels described include a hydrophilic material.

As used herein, the term “hydrogel” refers to a three-dimensional polymeric structure that is substantially insoluble in water, but which is capable of absorbing and retaining water (e.g., large quantities of water) to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. Hydrogels can contain over 99% water and may include natural or synthetic polymers, or a combination thereof. Hydrogels also possess a degree of flexibility very similar to natural tissue, due to their significant water content. A detailed description of suitable hydrogels may be found in published U.S. patent application 2010/0055733, herein incorporated by reference. By “hydrogel subunits” or “hydrogel precursors” is meant hydrophilic monomers, prepolymers, or polymers that can be crosslinked, or “polymerized”, to form a three-dimensional (3D) hydrogel network. In some embodiments, the alternating layers of polymeric gels described herein are hydrogels. Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO-PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl)cystamine (BACy), or PEG/polypropylene oxide (PPO). In embodiments, the hydrogel includes chemical crosslinks (e.g., intermolecular or intramolecular joining of two or more molecules by a covalent bond) and may be referred to as a chemical hydrogel. In embodiments, the hydrogel includes physical crosslinks (e.g., intermolecular or intramolecular joining of two or more molecules by a non-covalent bond) and may be referred to as a physical hydrogel. In embodiments, the physical hydrogel include one or more crosslinks including hydrogen bonds, hydrophobic interactions, and/or polymer chain entanglements.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive | Bioconjugate reactive | |
|---|---|---|
| group 1 (e.g., | group 2 (e.g., | |
| electrophilic | nucleophilic | |
| bioconjugate | bioconjugate | Resulting Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or |
| | | anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

As used herein, the term “control” or “control experiment” is used in accordance with its plain and ordinary meaning and refers to an experiment in which the subjects, cells, tissues, or reagents of the experiment are treated as in a parallel experiment except for omission of a procedure, reagent, or variable of the experiment. In some instances, the control is used as a standard of comparison in evaluating experimental effects. In embodiments, a control cell is the same cell type as the cell being examined, wherein the control cell does not include the variable or is subjected to conditions being examined.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

As used herein, a “plurality” refers to two or more.

As used herein the terms “automated” and “semi-automated” mean that the operations are performed by system programming or configuration with little or no human interaction once the operations are initiated, or once processes including the operations are initiated.

Provided herein are methods, systems, devices, and compositions for analyzing a sample in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

As used herein, the term “disease state” is used in accordance with its plain and ordinary meaning and refers to any abnormal biological state or aberration of a cell. The presence of a disease state may be identified by the same collection of biological constituents used to determine the cell's biological state. In general, a disease state will be detrimental to a biological system. A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). The methods provided herein may also be applicable to monitoring the disease state or states of a subject undergoing one or more therapies. Thus, provided herein, for example, are methods for determining or monitoring efficacy of a therapy or therapies (i.e., determining a level of therapeutic effect) upon a subject. In embodiments, the methods provided herein can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, i.e., a particular disease state or therapy, even at a stage where changes in protein activity are undetectable.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell. A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (i.e., biological targets of interest) or used for detection (i.e., probes containing oligonucleotide barcodes) in the methods and devices as described herein.

The terms “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

As used herein a “genetically modifying agent” is a substance that alters the genetic sequence of a cell following exposure to the cell, resulting in an agent-mediated nucleic acid sequence. In embodiments, the genetically modifying agent is a small molecule, protein, pathogen (e.g., virus or bacterium), toxin, oligonucleotide, or antigen. In embodiments, the genetically modifying agent is a virus (e.g., influenza) and the agent-mediated nucleic acid sequence is the nucleic acid sequence that develops within a T-cell upon cellular exposure and contact with the virus. In embodiments, the genetically modifying agent modulates the expression of a nucleic acid sequence in a cell relative to a control (e.g., the absence of the genetically modifying agent).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided a composition including: i) a biomolecule bound to a proximity probe, wherein the proximity probe includes an extended probe oligonucleotide including, from 5′ to 3′, a first primer binding sequence, a first barcode sequence, a first probe sequence, a complement of a second barcode sequence, and a complement of a second primer binding sequence; and ii) an oligonucleotide primer hybridized to the extended probe oligonucleotide, wherein the oligonucleotide primer includes, from 5′ to 3′, a first sequence complementary to the first primer binding sequence and a second sequence complementary to the complement of the second primer binding sequence.

In an aspect is provided a composition including: i) a biomolecule bound by a proximity probe, wherein the proximity probe includes an extended probe oligonucleotide including, from 5′ to 3′, a first primer binding sequence, a first barcode sequence, a first probe sequence, a complement of a second barcode sequence, a complement of a third probe sequence, a complement of a third barcode sequence, a complement of a fifth probe sequence, an internal cleavable site, and a complement of a second primer binding sequence; and ii) an oligonucleotide primer hybridized to the extended probe oligonucleotide, wherein the oligonucleotide primer includes, from 5′ to 3′, a first sequence complementary to the first primer binding sequence and a second sequence complementary to the complement of the second primer binding sequence.

In embodiments, the composition is in a cell. In embodiments, the cell is attached to a substrate. In embodiments, the cell is attached to the substrate via a bioconjugate reactive moiety. In embodiments, the composition is within a cell or tissue sample. In embodiments, the cell or tissue sample is cleared (e.g., digested) of proteins, lipids, or proteins and lipids. In embodiments, the cell or tissue sample is processed according to a known technique in the art, for example CLARITY (Chung K., et al. Nature 497, 332-337 (2013)), PACT-PARS (Yang B et al. Cell 158, 945-958 (2014).), CUBIC (Susaki E. A. et al. Cell 157, 726-739 (2014)., 18), ScaleS (Hama H., et al. Nat. Neurosci. 18, 1518-1529 (2015)), OPTIClear (Lai H. M., et al. Nat. Commun. 9, 1066 (2018)), Cₑ3D (Li W., et al. Proc. Natl. Acad. Sci. U.S.A. 114, E7321-E7330 (2017)), BABB (Dodt H. U. et al. Nat. Methods 4, 331-336 (2007)), iDISCO (Renier N., et al. Cell 159, 896-910 (2014)), uDISCO (Pan C., et al. Nat. Methods 13, 859-867 (2016)), FluoClearBABB (Schwarz M. K., et al. PLOS ONE 10, e0124650 (2015)), Ethanol-ECi (Klingberg A., et al. J. Am. Soc. Nephrol. 28, 452-459 (2017)), and PEGASOS (Jing D. et al. Cell Res. 28, 803-818 (2018)).

In an aspect is provided a kit. In embodiments, the kit includes a composition as described herein. In embodiments, the kit includes the reagents and containers useful for performing the methods as described herein. Generally, the kit includes one or more containers providing a composition and one or more additional reagents (e.g., a buffer suitable for polynucleotide extension and/or sequencing). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores).

In as aspect is provided a kit including the proximity probe and oligonucleotide primer of any one of the aspects and embodiments herein.

In embodiments, the oligonucleotide primer (i.e., the circularizable oligonucleotide) includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the circularizable oligonucleotide includes one or more LNA nucleotides. In embodiments, the sequence complementary to the first hybridization sequence and/or the second sequence complementary to the second hybridization sequence of the circularizable oligonucleotide includes one or more LNA nucleotides.

In embodiments, the first hybridization sequence (i.e., a first sequence complementary to the first primer binding sequence) of each oligonucleotide primer is greater than 30 nucleotides. In embodiments, the first hybridization sequence of each oligonucleotide primer is about 5 to about 35 nucleotides in length. In embodiments, the first hybridization sequence is about 12 to 15 nucleotides in length. In embodiments, the first hybridization sequence is about 35 to 40 nucleotides in length to maximize specificity. In embodiments, the first hybridization sequence is greater than 12 nucleotides in length. In embodiments, the first hybridization sequence is about 5, about 10, about 15, about 20, about 25, about 30, or about 35 nucleotides in length.

In embodiments, the second hybridization sequence (i.e., the second sequence complementary to the complement of the second primer binding sequence) of each oligonucleotide primer is greater than 30 nucleotides. In embodiments, the second hybridization sequence of each oligonucleotide primer is about 5 to about 35 nucleotides in length. In embodiments, the second hybridization sequence is about 12 to 15 nucleotides in length. In embodiments, the second hybridization sequence is about 35 to 40 nucleotides in length to maximize specificity. In embodiments, the second hybridization sequence is greater than 12 nucleotides in length. In embodiments, the second hybridization sequence is about 5, about 10, about 15, about 20, about 25, about 30, or about 35 nucleotides in length.

In embodiments, each oligonucleotide primer (e.g., each oligonucleotide primer of a plurality of oligonucleotides) includes one or more primer binding sequences (i.e., a sequence complementary to a primer, such as an amplification or sequencing primer) located between a 5′ end and a 3′ end of the oligonucleotide primer. In embodiments, the circularizable oligonucleotide includes a primer binding sequence.

In embodiments, the oligonucleotide primer (e.g., the circularizable oligonucleotide) includes about 50 to about 150 nucleotides. In embodiments, the circularizable oligonucleotide includes about 50 to about 300 nucleotides. In embodiments, the circularizable oligonucleotide includes about 50 to about 500 nucleotides. In embodiments, the circularizable oligonucleotide includes about or more than about 50, 75, 100, 125, 150, 175, 200, 250, 300, 350, 400, or 500 nucleotides. In embodiments, the circularizable oligonucleotide includes less than about 50, 75, 100, 125, 150, 175, 200, 250, 300, 350, 400, or 500 nucleotides.

In embodiments, the extended probe oligonucleotide includes about 50 to about 150 nucleotides. In embodiments, the extended probe oligonucleotide includes about 50 to about 300 nucleotides. In embodiments, the extended probe oligonucleotide includes about 50 to about 500 nucleotides. In embodiments, the extended probe oligonucleotide includes about or more than about 50, 75, 100, 125, 150, 175, 200, 250, 300, 350, 400, or 500 nucleotides. In embodiments, the extended probe oligonucleotide includes less than about 50, 75, 100, 125, 150, 175, 200, 250, 300, 350, 400, or 500 nucleotides.

In embodiments, the circularizable oligonucleotide includes at least one amplification primer binding sequence or at least one sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, the amplification primer binding sequence and/or sequencing primer binding sequence includes any one of the sequences (e.g., all or a portion thereof), or complement thereof, as described in Table 2. In embodiments, the amplification primer binding sequence includes any one of the sequences, or complement thereof, of SEQ ID NO:21 to SEQ ID NO:74. In embodiments, the sequencing primer binding sequence includes any one of the sequences, or complement thereof, of SEQ ID NO:21 to SEQ ID NO:74. In embodiments, the amplification primer binding sequence includes any one of the sequences, or complement thereof, of SEQ ID NO: 21, SEQ ID NO:22, SEQ ID NO:23, SEQ ID NO:25, SEQ ID NO:27, SEQ ID NO:48, or SEQ ID NO:53. In embodiments, the sequencing primer binding sequence includes any one of the sequences, or complement thereof, of SEQ ID NO: 21, SEQ ID NO:22, SEQ ID NO:23, SEQ ID NO:25, SEQ ID NO:27, SEQ ID NO:48, or SEQ ID NO:53. In embodiments, the amplification primer binding sequence includes any one of the sequences, or complement thereof, of SEQ ID NO: 27, SEQ ID NO:62, SEQ ID NO:37, SEQ ID NO:48, SEQ ID NO:22, SEQ ID NO:67, or SEQ ID NO:53. In embodiments, the sequencing primer binding sequence includes any one of the sequences, or complement thereof, of SEQ ID NO: 27, SEQ ID NO:62, SEQ ID NO:37, SEQ ID NO:48, SEQ ID NO:22, SEQ ID NO:67, or SEQ ID NO:53.

In embodiments, each oligonucleotide primer includes a barcode sequence. In embodiments, the circularizable oligonucleotide includes a barcode sequence. In embodiments, the extended probe oligonucleotide includes a barcode sequence.

In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a proximity probe conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide) can be associated with a target of interest by knowing, a priori, the target of interest, such as a gene or protein. In embodiments, the barcodes further include one or more sequences capable of specifically binding a gene or nucleic acid sequence of interest. For example, in embodiments, the barcode includes a sequence capable of hybridizing to mRNA, e.g., one containing a poly-T sequence (e.g., having several T's in a row, e.g., 4, 5, 6, 7, 8, or more T's).

In embodiments, the barcode is included as part of an oligonucleotide of longer sequence length, such as a primer or a random sequence (e.g., a random N-mer). In embodiments, the barcode contains random sequences to increase the mass or size of the oligonucleotide tag. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides. In embodiments, each barcode sequence is selected from a known set of barcode sequences.

In embodiments, the kit includes a microplate, and reagents for sample preparation and purification, amplification, and/or sequencing (e.g., one or more sequencing reaction mixtures). In embodiments, the kit includes for protein detection includes a plurality of proximity probes linked to an oligonucleotide (e.g., DNA-conjugated antibodies).

In embodiments, amplification reagents and other reagents may be provided in lyophilized form. In embodiments, amplification reagents and other reagents may be provided in a container that includes wells within which the lyophilized reagent may be reconstituted.

In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit further includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label. In embodiments, the kit includes a modified terminal deoxynucleotidyl transferase (TdT) enzyme.

In embodiments, the kit includes a cleaving agent (e.g., a cleaving agent for cleaving the internal cleavable site of the extended oligonucleotide probe). In embodiments, the cleaving agent is a restriction endonuclease. In embodiments, the cleavable site is cleaved as a result of enzymatic cleaving, for example, the activity of one or more restriction enzymes that recognize particular restriction site sequences in one or both strands of the cleavable site result in cleavage of the cleavable site. For example, in embodiments, the restriction site recognition sequence included in the cleavable site may include any one of the sequences listed in Table 1. In embodiments, the restriction enzyme recognition sequence included in the cleavable site is selected to be a “rare-cutting” restriction enzyme recognition sequence, e.g., a restriction enzyme that cuts with low frequency in any given genome. For example, Nod is a rare cutter with an eight-base recognition site, which will occur on average about once every 65,000 base pairs in a genome (assuming an average frequency of each type of canonical base of ¼). Other rare-cutting enzymes are known in the art and commercially available, including AbsI, AscI, BbvCI, CciNI, FseI, MreI, PaIAI, RigI, SdaI, and SgsI.

In embodiments, the kit includes an endonuclease (e.g., a nicking endonuclease). In embodiments, the endonuclease includes one or more endonucleases selected from the group consisting of Nb.BbvCI, Nb.BsmI, NbBsrDI, Nb.BtsI, Nt.AlwI, Nt.BbvCI, Nb.BssSI, Nt.BsmAI, Nt.BspQI, Nt.BstNBI, and Nt.CviPII. In embodiments, the endonuclease is Nb.BbvCI or Nt.BsmAI. In embodiments, the endonuclease is Nb.BbvCI. In embodiments, the endonuclease is Nt.BssmAI.

In embodiments, the kit includes an oligonucleotide complementary to a cleavable site (e.g., an oligonucleotide including a sequence complementary to the cleavable site, wherein the cleavable site includes an endonuclease recognition sequence). In embodiments, the kit includes an oligonucleotide including a sequence complementary to the endonuclease recognition sequence (e.g., the endonuclease recognition sequence of the first cleavable site).

In embodiments, the kit includes an exonuclease. In embodiments, the exonuclease is a 5′-3′ exonuclease. In embodiments, the 5′-3′ exonuclease is lambda exonuclease, or a mutant thereof.

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g., Therminator 7, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100.

In embodiments, the kit includes one or more sequencing reaction mixtures. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, coomassie blue, cresyl violet, DAPI, eosin, ethidium bromide, acid fuchsin, hematoxylin, Hoechst stains, iodine, methyl green, methylene blue, neutral red, Nile blue, Nile red, osmium tetroxide, propidium iodide, rhodamine, safranin, or any combination thereof.

### III. Methods

In an aspect is provided a method of forming an oligonucleotide including two barcode sequences. In embodiments, the method includes associating a first barcode with a first biomolecule and associating a second barcode with a second biomolecule. In embodiments, the method includes: a) contacting a first biomolecule with a first proximity probe, wherein the first proximity probe includes a first oligonucleotide including, from 5′ to 3′, a first primer binding sequence, a first barcode sequence, and a first probe sequence; b) contacting a second biomolecule with a second proximity probe, wherein the second proximity probe includes a second oligonucleotide including, from 5′ to 3′, a second primer binding sequence, a second barcode sequence, and a second probe sequence; c) hybridizing the first probe sequence of the first oligonucleotide to the second probe sequence of the second oligonucleotide and extending the first probe sequence with a polymerase to form a first extended oligonucleotide including, from 5′ to 3′, the first primer binding sequence, the first barcode sequence, the first probe sequence, a complement of the second barcode sequence, and a complement of the second primer binding sequence. In embodiments, prior to step a), the method includes obtaining a sample and optionally immobilizing the sample to a solid support. In embodiments, the method includes isolating the first extended oligonucleotide, amplifying the first extended oligonucleotide, and sequencing the first extended oligonucleotide.

In an aspect is provided a method of forming an oligonucleotide including two barcode sequences, the method including: a) contacting a first biomolecule with a first proximity probe, wherein the first proximity probe includes a first oligonucleotide including, from 5′ to 3′, a first primer binding sequence, a first barcode sequence, and a first probe sequence; b) contacting a second biomolecule with a second proximity probe, wherein the second proximity probe includes a second oligonucleotide including, from 5′ to 3′, a second primer binding sequence, a second barcode sequence, and a second probe sequence; c) hybridizing the first probe sequence of the first oligonucleotide to the second probe sequence of the second oligonucleotide and extending the first probe sequence with a polymerase to form a first extended oligonucleotide including, from 5′ to 3′, the first primer binding sequence, the first barcode sequence, the first probe sequence, a complement of the second barcode sequence, and a complement of the second primer binding sequence.

In embodiments, the first oligonucleotide, the second oligonucleotide, or both the first and the second oligonucleotide include one or more cleavable site(s). In embodiments, both the first and the second oligonucleotide include a first cleavable site. In embodiments, the cleavable site (e.g., the first cleavable site) is at or near the 5′ end of the first oligonucleotide, the second oligonucleotide, or both the first and the second oligonucleotides. In embodiments, the cleavable site (e.g., the first cleavable site) of the first oligonucleotide is 5′ of the first primer binding sequence, or wherein the cleavable site (e.g., the first cleavable site) of the second oligonucleotide is 5′ of the second primer binding sequence. In embodiments, the first cleavable site of the first oligonucleotide is 5′ of the first primer binding sequence, and the first cleavable site of the second oligonucleotide is 5′ of the second primer binding sequence. In embodiments, the second oligonucleotide includes a first cleavable site. In embodiments, the first cleavable site of the second oligonucleotide is 5′ of the second primer binding sequence.

In embodiments, the first oligonucleotide includes, from 5′ to 3′, a first primer binding sequence, a first barcode sequence, and a first probe sequence. In embodiments, the second oligonucleotide includes, from 5′ to 3′, a second primer binding sequence, a second barcode sequence, and a second probe sequence. In embodiments, the first oligonucleotide includes, from 5′ to 3′, a first cleavable site, a first primer binding sequence, a first barcode sequence, and a first probe sequence. In embodiments, the second oligonucleotide includes, from 5′ to 3′, a first cleavable site, a second primer binding sequence, a second barcode sequence, and a second probe sequence.

In embodiments, the method includes cleaving the cleavable site (e.g. the first cleavable site), amplifying the first extended oligonucleotide including the two barcode sequences, or complements thereof, to form amplification products, and detecting the amplification products (e.g., sequencing the amplification products). In embodiments, the two barcode sequences, or complements thereof, include the first barcode sequence and the complement of the second barcode sequence.

In embodiments, the method includes cleaving the cleavable site (e.g., the first cleavable site) and removing the second oligonucleotide (e.g., leaving behind a single-stranded extended oligonucleotide attached to the first proximity probe). In embodiments, cleaving includes contacting the cleavable site with a cleaving agent.

In embodiments, the method further includes detecting the first extended oligonucleotide (e.g., detecting via sequencing methods described herein, or for example, by fluorescent detection methods). In embodiments, the method further includes sequencing the two barcode sequences, or complements thereof, of the extended oligonucleotide (e.g., the first extended oligonucleotide). In embodiments, the method further includes sequencing the three barcode sequences, or complements thereof, of the extended oligonucleotide (e.g., the third extended oligonucleotide). In embodiments, the method further includes sequencing one barcode sequence, or complement thereof. In embodiments, the method further includes sequencing two barcode sequences, or complements thereof. In embodiments, the method further includes sequencing three or more barcode sequences, or complements thereof.

In embodiments, the method further includes hybridizing an oligonucleotide primer to the first extended oligonucleotide, wherein the oligonucleotide primer includes, from 5′ to 3′, a first sequence complementary to the first primer binding sequence and a second sequence complementary to the complement of the second primer binding sequence, and extending the second sequence along the extended oligonucleotide to generate a complementary sequence, and ligating the complementary sequence to first sequence of the oligonucleotide primer to form a circular oligonucleotide including the complement of the first barcode sequence and the second barcode sequence. In embodiments, the method further includes amplifying the circular oligonucleotide by extending an amplification primer hybridized to the circular oligonucleotide with a strand-displacing polymerase, wherein the amplification primer extension generates an extension product including multiple complements of the circular oligonucleotide. In embodiments, the method further includes sequencing the circular oligonucleotide.

In embodiments, the first biomolecule and the second biomolecule are different biomolecules (e.g., a CD2 protein and a CD58 protein). In embodiments, the first biomolecule and the second biomolecule are the same biomolecule. In embodiments, the first proximity probe and the second proximity probe contact the same biomolecule (e.g., the first biomolecule and the second biomolecule are different epitopes on the same biomolecule, such as the same protein). In embodiments, the first proximity probe and the second proximity probe contact different biomolecules (e.g., the first biomolecule and the second biomolecule are different biomolecules, such as different proteins). In embodiments, the first biomolecule and the second biomolecule are different biomolecules. In embodiments, the first biomolecule and the second biomolecule are the same biomolecule (e.g., the first biomolecule is a first epitope and the second biomolecule is a second epitope, wherein the first and second epitope are on the same protein).

In embodiments, the second oligonucleotide includes, from 5′ to 3′, a second primer binding sequence, a second internal cleavable site, a third probe sequence, a second barcode sequence, and a second probe sequence, and the first extended oligonucleotide includes, from 5′ to 3′, the first primer binding sequence, the first barcode sequence, the first probe sequence, a complement of the second barcode sequence, a complement of the third probe sequence, a cleavable complement of the second internal cleavable site, and a complement of the second primer binding sequence. In embodiments, the method further includes d) cleaving the second internal cleavable site of the second oligonucleotide and the cleavable complement of the second internal cleavable site of the first extended oligonucleotide, thereby forming a cleaved second oligonucleotide and a cleaved first extended oligonucleotide, and removing the cleaved second oligonucleotide. In embodiments, the method further includes d) extending the second oligonucleotide with a polymerase to form a second extended oligonucleotide including, from 5′ to 3′, the second primer binding sequence, the second internal cleavable site, the third probe sequence, the second barcode sequence, the second probe sequence, a complement of the first barcode sequence, and the second primer binding sequence. In embodiments, the method further includes cleaving the second internal cleavable site of the second extended oligonucleotide and the cleavable complement of the second internal cleavable site of the first extended oligonucleotide, thereby forming a cleaved second extended oligonucleotide and a cleaved first extended oligonucleotide, and removing the cleaved second extended oligonucleotide. In embodiments, the cleaved first extended oligonucleotide includes, from 5′ to 3′, the first primer binding sequence, the first barcode sequence, the first probe sequence, a complement of the second barcode sequence, and the complement of the third probe sequence.

In embodiments, the method further includes: e) contacting a third biomolecule with a third proximity probe, wherein the third proximity probe includes a third oligonucleotide including, from 5′ to 3′, the second primer binding sequence, the second internal cleavable site, a fifth probe sequence, a third barcode sequence, and a fourth probe sequence; and f) hybridizing the complement of the third probe sequence of the cleaved first extended oligonucleotide to the fourth probe sequence of the third oligonucleotide and extending the complement of the third probe sequence with a polymerase to form a third extended oligonucleotide including, from 5′ to 3′, the first primer binding sequence, the first barcode sequence, the first probe sequence, the complement of the second barcode sequence, the complement of the third probe sequence, a complement of the third barcode sequence, a complement of the fifth probe sequence; the cleavable complement of the second internal cleavable site, and the complement of the second primer binding sequence. In embodiments, the method further includes g) extending the third oligonucleotide with the polymerase to form a fourth extended oligonucleotide including, from 5′ to 3′, the second primer binding sequence, the second internal cleavable site, the fifth probe sequence, the third barcode sequence, the fourth probe sequence, a complement of the first barcode sequence, a complement of the first probe sequence, the complement of the first barcode sequence, and the complement of the first primer binding sequence. In embodiments, the third oligonucleotide includes the first cleavable site at or near the 5′ end. In embodiments, the first cleavable site of the third oligonucleotide is 5′ of the second primer binding sequence. In embodiments, the method includes cleaving the first cleavable site of the third oligonucleotide, amplifying the third extended oligonucleotide including the three barcode sequences, or complements thereof, to form amplification products, and sequencing the amplification products. In embodiments, the method further includes detecting the third extended oligonucleotide. In embodiments, the method further includes cleaving the first cleavable site at or near the 5′ end of the third oligonucleotide and removing the third oligonucleotide. In embodiments, the method further includes cleaving the first cleavable site at or near the 5′ end of the third oligonucleotide, removing the fourth extended oligonucleotide, and detecting the third extended oligonucleotide.

In an aspect is provided a method of forming an oligonucleotide including at least three (e.g., at least three barcode sequences, or more than three barcode sequences) barcode sequences. In embodiments, the method includes: a) contacting a first biomolecule with a first proximity probe, wherein the first proximity probe includes a first oligonucleotide including, from 5′ to 3′, a first primer binding sequence, a first barcode sequence, and a first probe sequence; b) contacting a second biomolecule with a second proximity probe, wherein the second proximity probe includes a second oligonucleotide including, from 5′ to 3′, a second primer binding sequence, a second internal cleavable site, a third probe sequence, a second barcode sequence, and a second probe sequence; c) contacting a third biomolecule with a third proximity probe, wherein the third proximity probe includes a third oligonucleotide including, from 5′ to 3′, the second primer binding sequence, the second internal cleavable site, a fifth probe sequence, a third barcode sequence, and a fourth probe sequence; d) hybridizing the first probe sequence of the first oligonucleotide to the second probe sequence of the second oligonucleotide, and extending the first probe sequence with a polymerase to form a first extended oligonucleotide including, from 5′ to 3′, the first primer binding sequence, the first barcode sequence, the first probe sequence, a complement of the second barcode sequence, a complement of the third probe sequence, a cleavable complement of the second internal cleavable site, and a complement of the second primer binding sequence; e) cleaving the second internal cleavable site of the second oligonucleotide (e.g., of the extended second oligonucleotide, also referred to as a second extended oligonucleotide) and the cleavable complement of the second internal cleavable site of the first extended oligonucleotide, thereby forming a cleaved second oligonucleotide and a cleaved first extended oligonucleotide, and removing the second oligonucleotide; and f) hybridizing the complement of the third probe sequence of the cleaved first extended oligonucleotide to the fourth probe sequence of the third oligonucleotide and extending the complement of the third probe sequence with a polymerase to form a third extended oligonucleotide including, from 5′ to 3′, the first primer binding sequence, the first barcode sequence, the first probe sequence, the complement of the second barcode sequence, the complement of the third probe sequence, a complement of the third barcode sequence, a complement of the fifth probe sequence, the cleavable complement of the second internal cleavable site, and the complement of the second primer binding sequence.

In an aspect is provided a method of incorporating one or more additional barcode sequences into a first extended oligonucleotide, wherein the first extended oligonucleotide includes at least two barcode sequences. In embodiments, the method includes: a) contacting a first biomolecule with a first proximity probe, wherein the first proximity probe includes the first extended oligonucleotide including a first primer binding sequence, at least two barcode sequences (e.g., at least a first barcode sequence and a second barcode sequence), and a first probe sequence; b) contacting a second biomolecule with a second proximity probe, wherein the second proximity probe includes a second oligonucleotide including, from 5′ to 3′, a second primer binding sequence, a second internal cleavable site, a third probe sequence, a barcode sequence (e.g., a third barcode sequence), and a second probe sequence; c) hybridizing the first probe sequence of the first extended oligonucleotide to the second probe sequence of the second oligonucleotide, and extending the probe sequence of the first extended oligonucleotide with a polymerase to form a second extended oligonucleotide including the first primer binding sequence, the at least two barcode sequences of the first extended oligonucleotide, the first probe sequence, a complement of the third probe sequence, a complement of the barcode sequence of the second oligonucleotide, a complement of the second probe sequence, a cleavable complement of the second internal cleavable site, and a complement of the second primer binding sequence. In embodiments, the method further includes cleaving the second internal cleavable site of the second oligonucleotide and the cleavable complement of the second internal cleavable site of the second extended oligonucleotide, and removing the second oligonucleotide. In embodiments, the method further includes extending the second oligonucleotide to form a third extended oligonucleotide, including, from 5′ to 3′, the second primer binding sequence, the second internal cleavable site, the third probe sequence, the third barcode sequence, the second probe sequence, a complement of the first barcode sequence, a complement of the first probe sequence, the complement of the first barcode sequence, and the complement of the first primer binding sequence. In embodiments, the second oligonucleotide includes a first cleavable site at or near the 5′ end. In embodiments, the first cleavable site of the second oligonucleotide is 5′ of the second primer binding sequence. In embodiments, the method includes cleaving the first cleavable site of the second oligonucleotide, amplifying the second extended oligonucleotide including the three barcode sequences, or complements thereof, to form amplification products, and detecting (e.g., sequencing) the amplification products. In embodiments, the method further includes detecting the second extended oligonucleotide. In embodiments, the method further includes cleaving the first cleavable site at or near the 5′ end of the second oligonucleotide and removing the second oligonucleotide. In embodiments, the method further includes cleaving the first cleavable site at or near the 5′ end of the second oligonucleotide, removing the third extended oligonucleotide, and detecting the second extended oligonucleotide. In embodiments, the method is repeated for at least one additional barcode sequence (e.g., the extended oligonucleotide including one additional barcode sequence is hybridized to another probe oligonucleotide including a barcode sequence).

In embodiments, the first oligonucleotide, the second oligonucleotide, and the third oligonucleotide include one or more first cleavable site(s). In embodiments, the first oligonucleotide, the second oligonucleotide, or the third oligonucleotide include one or more first cleavable site(s). In embodiments, both the second and the third oligonucleotide include a first cleavable site. In embodiments, the cleavable site (e.g., the first cleavable site) is at or near the 5′ end of the first oligonucleotide, the second oligonucleotide, or the third oligonucleotide. In embodiments, the cleavable site (e.g., the first cleavable site) of the first oligonucleotide is 5′ of the first primer binding sequence. In embodiments, the first cleavable site of the second oligonucleotide is 5′ of the second primer binding sequence. In embodiments, the first cleavable site of the third oligonucleotide is 5′ of the second primer binding sequence.

In embodiments, cleaving the cleavable site provides a remnant sequence (e.g., leaves behind a probe sequence at the 3′ end of the oligonucleotide) that is then capable of hybridizing to a complementary probe sequence of a different oligonucleotide, wherein the oligonucleotides are conjugated to different proximity probes.

As used herein, “probe oligonucleotide” refers to the oligonucleotide attached, conjugated, or otherwise linked to a proximity probe. In embodiments, the probe oligonucleotide is a single-stranded oligonucleotide. In embodiments, the probe oligonucleotide is partially double-stranded. In embodiments, the 3′ end of the probe oligonucleotide is single-stranded. In embodiments, the proximity probe is covalently linked via a linker to the probe oligonucleotide. In embodiments, the linker includes one or more cleavable sites. In embodiments, the probe oligonucleotide includes the linker (i.e., the probe linker) covalently attached to the proximity probe.

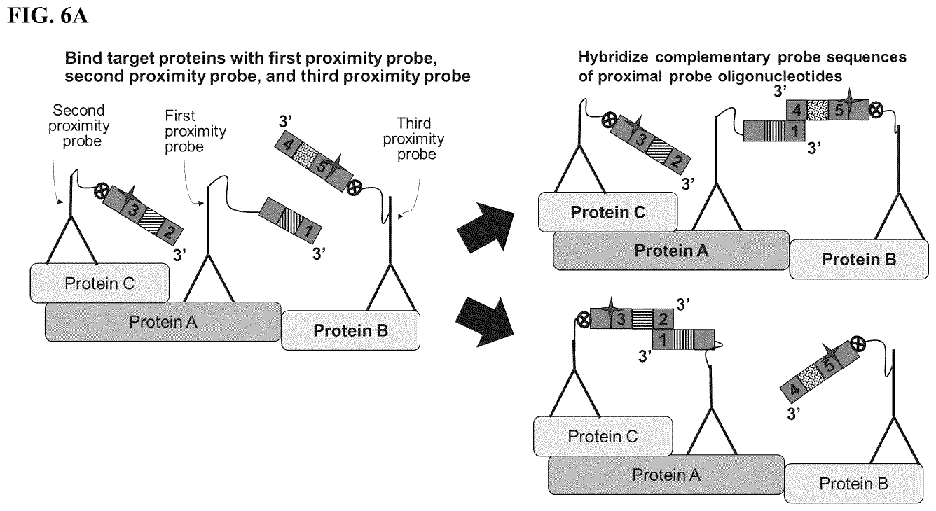

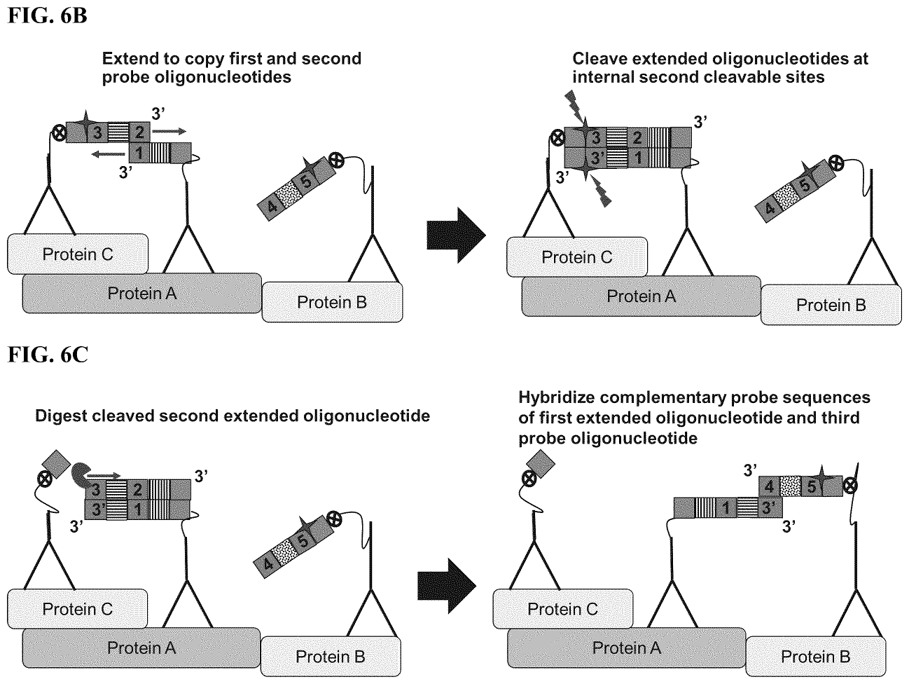

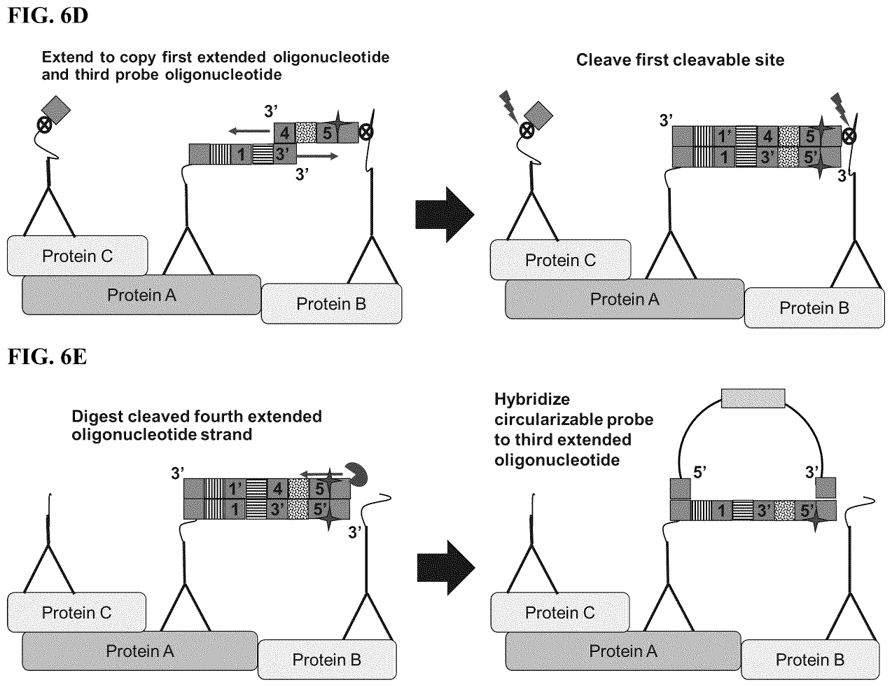

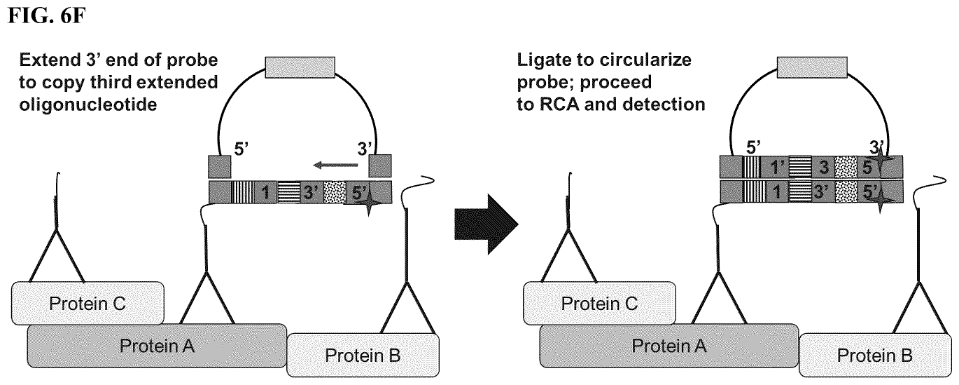

In embodiments, cleaving the internal cleavable site (e.g., the second internal cleavable sire, or cleavable complement thereof) of the second or third probe oligonucleotide forms a cleaved probe oligonucleotide. For example, cleaving the cleavable complement of the second internal cleavable site of a first extended oligonucleotide and cleaving the second internal cleavable site of a second extended oligonucleotide, wherein the first extended oligonucleotide and the second extended oligonucleotide are at least partially duplexed, generates a cleaved first extended oligonucleotide including a probe sequence, or complement thereof, at the 3′ end of the cleaved first extended oligonucleotide, and a cleaved second extended oligonucleotide including a probe sequence, or complement thereof, at the 5′ end of the cleaved second extended oligonucleotide (see, e.g., FIG. 6B).

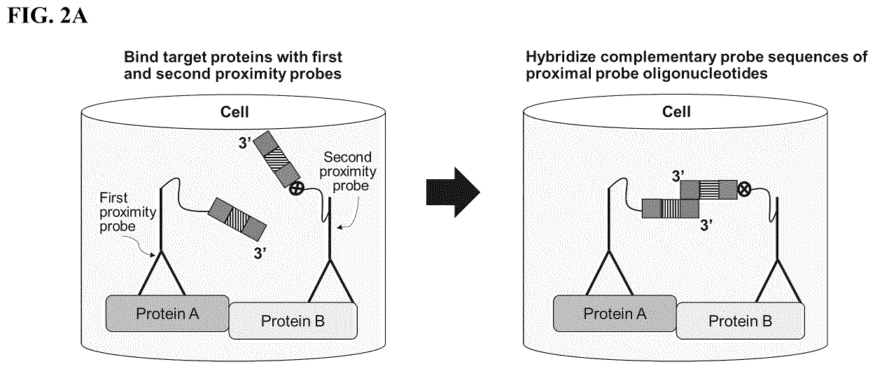

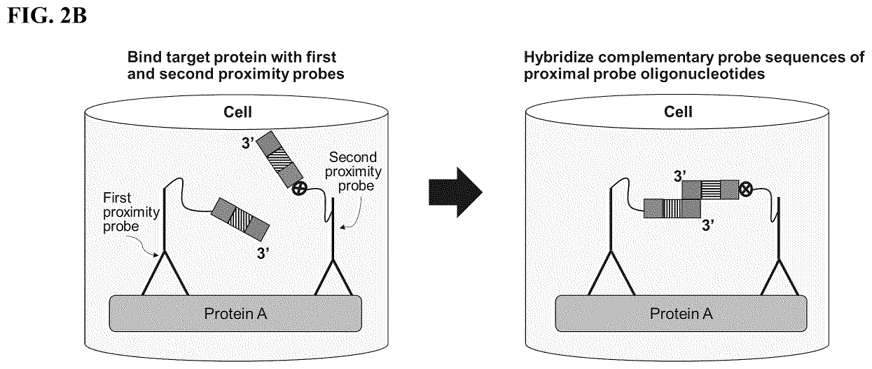

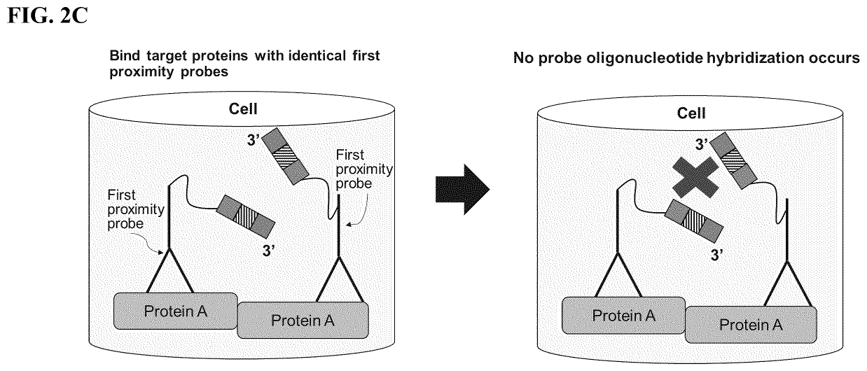

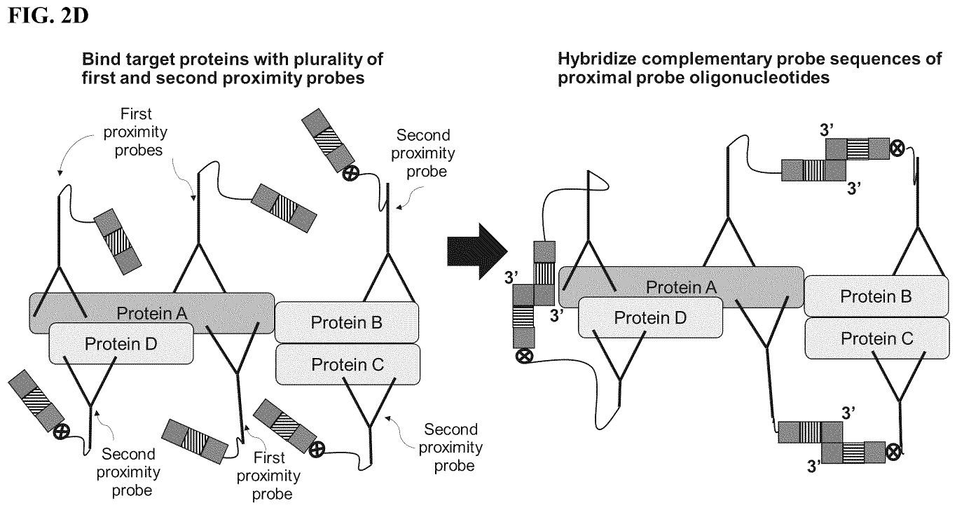

As described herein, and illustrated for example in FIGS. 2A-2B, 2D, 6A, and 6C, the probe sequence at the 3′ end of a probe oligonucleotide (e.g., the probe sequence at the 3′ end of a first probe oligonucleotide, a second probe oligonucleotide, a third probe oligonucleotide, or additional probe oligonucleotides) allows for a first probe oligonucleotide to hybridize to a proximal second probe oligonucleotide, wherein the probe sequence of the first probe oligonucleotide and the probe sequence of the second probe oligonucleotide are complementary. In embodiments, the first probe oligonucleotide includes a first probe sequence at the 3′ end of the first probe oligonucleotide, and the second probe oligonucleotide (or third probe oligonucleotide) contains a second probe sequence at the 3′ end of the second probe oligonucleotide and a third probe sequence located 5′ of the second probe sequence (see, e.g., FIG. 6A). As described herein, and illustrated in FIGS. 6B-6D, following hybridization and extension of the first probe oligonucleotide, a complement of the third probe sequence is incorporated into the first extended probe oligonucleotide. Following cleavage of the second internal cleavable site, and complement thereof, the complement of the third probe sequence may then hybridize to an additional proximal probe oligonucleotide (e.g., the complement of the third probe sequence of the cleaved first extended oligonucleotide may hybridize to a 3′ probe sequence of a third probe oligonucleotide, as illustrated in FIG. 6C).

The two components of the proximity probe (e.g., a biomolecule-binding domain and a probe oligonucleotide) are joined together either directly through a bond or indirectly through a linking group. Where linking groups are employed, such groups may be chosen to provide for covalent attachment of the probe oligonucleotide and biomolecule-binding domains through the linking group, as well as maintain the desired binding affinity of the biomolecule-binding domain for its target biomolecule. Linking groups of interest may vary widely depending on the biomolecule-binding domain. The linking group (i.e., the linker), when present, is in many embodiments biologically inert. A variety of linking groups are known to those of skill in the art and find use in the subject proximity probes. In embodiments, the linking group is at least between 50 Daltons to 1,000 Daltons, 1,000 Daltons to 10,000 Daltons, or 10,000 Daltons to 100,000 Daltons. In embodiments, the linking group is generally at least about 50 Daltons, 100 Daltons, 300 Daltons, 500 Daltons, 1000 Daltons, 2000 Daltons, 3000 Daltons, 6000 Daltons, 12,000 Daltons, 30,000 Daltons, or larger, for example up to 1,000,000 Daltons. In embodiments, the linker may contain a spacer. Generally, such linkers will include a spacer group terminated at either end with a reactive functionality capable of covalently bonding to the probe oligonucleotide or biomolecule-binding moieties. Spacer groups of interest may include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides.

Specific linker groups that may find use in the subject proximity probes include heterofunctional compounds, such as azidobenzoyl hydrazide, N-[4-(p-azidosalicylamino)butyl]-3′-[2′-pyridyldithio]propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl[4-azidophenyl]-1,3′-dithiopropionate, N-succinimidyl[4-iodoacetyl]aminobenzoate, glutaraldehyde, and succinimidyl-4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and the like.

In embodiments, the method further includes detecting the first extended oligonucleotide. In embodiments, the method further includes detecting the second extended oligonucleotide. In embodiments, the method further includes removing the second extended oligonucleotide, prior to detecting the first extended oligonucleotide. In embodiments, the method further includes removing the first extended oligonucleotide, prior to detecting the second extended oligonucleotide. In embodiments, both the first extended oligonucleotide and the second extended oligonucleotide (e.g., a duplex of both extended oligonucleotides) are isolated from one or more cells prior to detecting.

In embodiments, the method further includes detecting the third extended oligonucleotide. In embodiments, the method further includes detecting the fourth extended oligonucleotide. In embodiments, the method further includes removing the fourth extended oligonucleotide, prior to detecting the third extended oligonucleotide. In embodiments, the method further includes removing the third extended oligonucleotide, prior to detecting the fourth extended oligonucleotide. In embodiments, both the third extended oligonucleotide and the fourth extended oligonucleotide (e.g., a duplex of both extended oligonucleotides) are isolated from one or more cells prior to detecting.

In embodiments, the second oligonucleotide, the third oligonucleotide, or both of the second and third oligonucleotides include a cleavable site at or near the 5′ end. In embodiments, the first oligonucleotide, the second oligonucleotide, the third oligonucleotide, or each of the first, second, and third oligonucleotides include a cleavable site at or near the 5′ end.

In embodiments, the first proximity probe binds to the first biomolecule with a specific binding affinity (e.g., a specific dissociation constant K_{D}). In embodiments, the second proximity probe binds to the second biomolecule with a specific binding affinity (e.g., a specific dissociation constant K_{D}). In embodiments, the third proximity probe binds to the third biomolecule with a specific binding affinity (e.g., a specific dissociation constant K_{D}). The equilibrium dissociation constant, K_{D}, is a measure of the strength of an interaction between a biomolecule and its binding partner. In embodiments, the proximity probe binds to the first molecule with a K_{D} in the low micromolar (10⁻⁶) to nanomolar (10⁻⁷ to 10⁻¹) range. In embodiments, the proximity probe binds to the first molecule with a K_{D} in the low nanomolar range (10⁻¹). In embodiments, the proximity probe binds to the first molecule with a K_{D} in the picomolar (10⁻¹²) range. In embodiments, the proximity probe binds to the first molecule with a K_{D} of at least 10⁻⁹ nM. In embodiments, the proximity probe binds to the first molecule with a K_{D} of at least 10⁻¹² nM.

In embodiments, specific binding entails a binding affinity, expressed as a KD (such as a KD measured by surface plasmon resonance at an appropriate temperature, such as 37° C.). In embodiments, the KD of a specific binding interaction is less than about 100 nM, 50 nM, 10 nM, 1 nM, 0.05 nM, or lower. In embodiments, the KD of a specific binding interaction is about 0.01-100 nM, 0.1-50 nM, or 1-10 nM. In embodiments, the KD of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically a specific or selective reaction will be at least twice background signal to noise and more typically more than 10 to 100 times greater than background.

In embodiments, the method includes cleaving the cleavable site at or near the 5′ end of the third oligonucleotide, amplifying the extended oligonucleotide including the three barcode sequences, or complements thereof, to form amplification products, and detecting (e.g., sequencing) the amplification products. In embodiments, the method includes cleaving the cleavable site at or near the 5′ end of the second and third oligonucleotides, amplifying the extended oligonucleotide including the three barcode sequences, or complements thereof, to form amplification products, and detecting (e.g., sequencing) the amplification products. In embodiments, the method includes cleaving the cleavable site at or near the 5′ end of each of the oligonucleotides, amplifying the extended oligonucleotide including the three barcode sequences, or complements thereof, to form amplification products, and sequencing the amplification products. In embodiments, following cleavage of the cleavable site at or near the 5′ end of each of the oligonucleotide, the oligonucleotide is removed.

In embodiments, the cleaved oligonucleotide (e.g., the oligonucleotide with a free 5′ end) is removed by an exonuclease enzyme (e.g., contacting the oligonucleotide with a free 5′ end with an enzyme capable of digesting 5′ ends). In embodiments, the exonuclease enzyme is a 3′-5′ exonuclease. In embodiments, the exonuclease enzyme is a 5′-3′ exonuclease. In embodiments, the 3′-5′ exonuclease is exonuclease I, exonuclease T, a proofreading polymerase, or a mutant thereof. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading” activity. In embodiments, the proofreading polymerase is a phi29 polymerase, or mutant thereof. In embodiments, the 5′-3′ exonuclease is lambda exonuclease, or a mutant thereof.

In embodiments, removing the cleaved oligonucleotide (e.g., the oligonucleotide with a free 5′ end) includes incubation in a denaturant as described herein, for example, wherein the denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. Incubation in a denaturant should only remove the cleaved oligonucleotide and not remove the bound proximity probes from the biomolecule(s). Optimization of denaturant conditions may be performed to identify conditions suitable for selective denaturation. In embodiments, the reaction conditions are modified to denaturing conditions by i) increasing the temperature, ii) contacting the oligonucleotide with a chemical denaturant, or iii) a combination thereof.

The one or more cleavable sites may include a modified nucleotide, ribonucleotide, or a sequence containing a modified or unmodified nucleotide that is specifically recognized by a cleavage agent. The cleavable site(s) may be deoxyuracil triphosphate (dUTP), deoxy-8-Oxo-guanine triphosphate (d-8-oxoG), or other modified nucleotide(s), such as those described, for example, in US 2012/0238738, which is incorporated herein by reference for all purposes. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more ribonucleotides. In embodiments, the cleavable site includes 2 to 5 ribonucleotides. In embodiments, the cleavable site includes one ribonucleotide. In embodiments, the cleavable sites can be cleaved at or near a modified nucleotide or bond by enzymes or chemical reagents, collectively referred to here and in the claims as “cleaving agents.” Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In embodiments, when the modified nucleotide is a ribonucleotide, the cleavable site can be cleaved with an endoribonuclease. In embodiments, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In embodiments, the cleaving agent is an endonuclease enzyme such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In embodiments, the cleaving agent includes a restriction endonuclease, including, for example a type IIS restriction endonuclease. In embodiments, the cleaving agent is an exonuclease (e.g., RecBCD), restriction nuclease, endoribonuclease, exoribonuclease, or RNase (e.g., RNAse I, II, or III). In embodiments, the cleaving agent is a restriction enzyme. In embodiments, the cleaving agent includes a glycosylase and one or more suitable endonucleases. In embodiments, cleavage is performed under alkaline (e.g., pH greater than 8) buffer conditions at between 40° C. to 80° C.

TABLE 1
Restriction site sequences and corresponding
restriction enzymes (the “|” denotes the location
of the cleavage site following contact with the
restriction enzyme)

| Restriction Site Sequence | Restriction Enzyme |
|---|---|
| GACGT\|C | Aat II |
| CG\|CG | Acc II |
| T\|CCGGA | Aor13H I |
| AGC\|GCT | Aor51H I |
| TT\|CGAA | BspT104 I |
| G\|CGCGC | BssH II |
| AT\|CGAT | Cla I |
| C\|GGCCG | Eco52 I |
| C\|CGG | Hap II |
| GCG\|C | Hha I |
| A\|CGCGT | Mlu I |
| GCC\|GGC | Nae I |
| GC\|GGCCGC | Not I |
| TCG\|CGA | Nru I |
| TGC\|GCA | Nsb I |
| G\|TCGAC | Sal I |
| CCC\|GGG | Sma I |
| TAC\|GTA | SnaB I |
| A\|GATCT | Bgl II |

In embodiments, the method includes cleaving the cleavable site at or near the 5′ end of the third oligonucleotide and removing the third oligonucleotide. In embodiments, the method includes cleaving the cleavable site located upstream of the primer binding sequence, or complement thereof, of the third oligonucleotide and removing the third oligonucleotide. In embodiments, the method includes cleaving the cleavable site located upstream of the barcode sequence, or complement thereof, of the third oligonucleotide and removing the third oligonucleotide. In embodiments, the method includes cleaving the cleavable site at or near the 5′ end of each of the second and third oligonucleotides and removing the second and third oligonucleotides. In embodiments, the method includes cleaving the cleavable site located upstream of the primer binding sequence, or complement thereof, of each of the second and third oligonucleotides and removing the second and third oligonucleotides. In embodiments, the method includes cleaving the cleavable site located upstream of the barcode sequence, or complement thereof, of each of the second and third oligonucleotides and removing the second and third oligonucleotides.

In embodiments, cleaving the first cleavable site (e.g., the cleavable site at the 5′ end of the first probe oligonucleotide) includes contacting the first cleavable site with a nicking endonuclease. In embodiments, the method further includes contacting the first cleavable site with a complementary sequence (e.g., an oligonucleotide including a sequence complementary to the first cleavable site, wherein the first cleavable site includes a nicking endonuclease recognition sequence), thereby forming a double-stranded recognition sequence. These nicking endonucleases typically recognize non-palindromes. They can be bona fide nicking enzymes, such as frequent cutter Nt.CviPII and Nt.CviQII, or rare-cutting homing endonucleases I-BasI and I-HmuI, both of which recognize a degenerate 24-bp sequence. As well, isolated large subunits of heterodimeric Type IIS restriction endonucleases such as BtsI, BsrDI and BstNBI/BspD6I display nicking activity. Thus, properties of restriction endonucleases that make double-strand cuts may be retained by engineering variants of these enzymes such that they make single-strand breaks. In various embodiments, recognition sequence-specific nicking endonucleases are used as cleavage agents that cleave only a single-strand of double-stranded DNA at a cleavage site. Nicking endonucleases useful in various embodiments of methods and compositions described herein include Nb.BbvCI, Nb.BsmI, Nb.BsrDI, Nb.BtsI, Nt.AlwI, Nt.BbvCI, Nt.BsmAI, Nt.BspQI, Nt.BstNBI, and Nt.CviPII, used either alone or in various combinations. In various embodiments, nicking endonucleases that cleave outside of their recognition sequence, e.g., Nb.BsrDI, Nb.BtsI, Nt.AlwI, Nt.BsmAI, Nt.BspQI, Nt.BstNBI, and Nt.CviPII, are used. In some instances, nicking endonucleases that cut within their recognition sequences, e.g. Nb.BbvCI, Nb.BsmI, or Nt.BbvCI are used. Recognition sites for the various specific cleavage agents used herein, such as the nicking endonucleases, comprise a specific nucleic acid sequence.

The nickase Nb.BbvCI (New England Biolabs, Ipswich, Mass.) nicks at the following cleavage site with respect to its recognition site (with “I” specifying the nicking (cleavage) site and “N” representing any nucleoside, e.g. one of C, A, G or T): 5′-CCTCAGC-3′ (SEQ ID NO:1) and 3′-GGAGTICG-5′ (SEQ ID NO:2). The nickase Nb.BsmI (New England Biolabs, Ipswich, Mass.) nicks at the following cleavage site with respect to its recognition site: 5′-GAATGCN-3′ (SEQ ID NO:3) and 3′-CTTACIGN-5′ (SEQ ID NO:4). The nickase Nb.BsrDI (New England Biolabs, Ipswich, Mass.) nicks at the following cleavage site with respect to its recognition site: 5′-GCAATGNN-3′ (SEQ ID NO:5) and 3′-CGTTACINN-5′ (SEQ ID NO:6). The nickase Nb.BtsI (New England Biolabs, Ipswich, Mass.) nicks at the following cleavage site with respect to its recognition site: 5′-GCAGTGNN-3′ (SEQ ID NO:7) and 3′-CGTCACINN-5′ (SEQ ID NO:8). The nickase Nt.AlwI (New England Biolabs, Ipswich, Mass.) nicks at the following cleavage site with respect to its recognition site: 5′-GGATCNNNNIN-3′ (SEQ ID NO:9) and 3′-CCTAGNNNNN-5′ (SEQ ID NO:10). The nickase Nt.BbvCI (New England Biolabs, Ipswich, Mass.) nicks at the following cleavage site with respect to its recognition site: 5′-CCITCAGC-3′ (SEQ ID NO:11) and 3′-GGAGTCG-5′ (SEQ ID NO:12). The nickase Nt.BsmAI (New England Biolabs, Ipswich, Mass.) nicks at the following cleavage site with respect to its recognition site: 5′-GTCTCNIN-3′ (SEQ ID NO:13) and 3′-CAGAGNN-5′ (SEQ ID NO.: 14). The nickase Nt.BspQI (New England Biolabs, Ipswich, Mass.) nicks at the following cleavage site with respect to its recognition site: 5′-GCTCTTCNI-3′ (SEQ ID NO.: 15) and 3′-CGAGAAGN-5′ (SEQ ID NO:16). The nickase Nt.BstNBI (New England Biolabs, Ipswich, Mass.) nicks at the following cleavage site with respect to its recognition site: 5′-GAGTCNNNNIN-3′ (SEQ ID NO:17) and 3′-CTCAGNNNNN-5′ (SEQ ID NO:18). The nickase Nt.CviPII (New England Biolabs, Ipswich, Mass.) nicks at the following cleavage site with respect to its recognition site (wherein D denotes A or G or T and wherein H denotes A or C or T: 5′-ICCD-3′ (SEQ ID NO:19) and 3′-GGH-5′ (SEQ ID NO:20).

In embodiments, the endonuclease includes one or more endonucleases selected from the group consisting of Nb.BbvCI, Nb.BsmI, NbBsrDI, Nb.BtsI, Nt.AlwI, Nt.BbvCI, Nb.BssSI, Nt.BsmAI, Nt.BspQI, Nt.BstNBI, and Nt.CviPII. In embodiments, the endonuclease includes Nb.BbvCI. In embodiments, the endonuclease is Nb.BbvCI. In embodiments, the endonuclease is Nt.BsmAI.

In embodiments, the double-stranded recognition sequence includes SEQ ID NO:1 and SEQ ID NO:2. In embodiments, the double-stranded recognition sequence includes SEQ ID NO:3 and SEQ ID NO:4. In embodiments, the double-stranded recognition sequence includes SEQ ID NO:5 and SEQ ID NO:6. In embodiments, the double-stranded recognition sequence includes SEQ ID NO:7 and SEQ ID NO:8. In embodiments, the double-stranded recognition sequence includes SEQ ID NO:9 and SEQ ID NO:10. In embodiments, the double-stranded recognition sequence includes SEQ ID NO:11 and SEQ ID NO:12. In embodiments, the double-stranded recognition sequence includes SEQ ID NO:13 and SEQ ID NO:14. In embodiments, the double-stranded recognition sequence includes SEQ ID NO:15 and SEQ ID NO:16. In embodiments, the double-stranded recognition sequence includes SEQ ID NO:17 and SEQ ID NO:18. In embodiments, the double-stranded recognition sequence includes SEQ ID NO:19 and SEQ ID NO:20.

In embodiments, the double-stranded recognition sequence includes SEQ ID NO:1 duplexed to SEQ ID NO:2. In embodiments, the double-stranded recognition sequence includes SEQ ID NO:3 duplexed to SEQ ID NO:4. In embodiments, the double-stranded recognition sequence includes SEQ ID NO:5 duplexed to SEQ ID NO:6. In embodiments, the double-stranded recognition sequence includes SEQ ID NO:7 duplexed to SEQ ID NO:8. In embodiments, the double-stranded recognition sequence includes SEQ ID NO:9 duplexed to SEQ ID NO:10. In embodiments, the double-stranded recognition sequence includes SEQ ID NO:11 duplexed to SEQ ID NO:12. In embodiments, the double-stranded recognition sequence includes SEQ ID NO:13 duplexed to SEQ ID NO:14. In embodiments, the double-stranded recognition sequence includes SEQ ID NO:15 duplexed to SEQ ID NO:16. In embodiments, the double-stranded recognition sequence includes SEQ ID NO:17 duplexed to SEQ ID NO:18. In embodiments, the double-stranded recognition sequence includes SEQ ID NO:19 duplexed to SEQ ID NO:20.

In embodiments, the endonuclease includes one or more endonucleases selected from the group consisting of Nb.BbvCI, Nb.BsmI, NbBsrDI, Nb.BtsI, Nt.AlwI, Nt.BbvCI, Nb.BssSI, Nt.BsmAI, Nt.BspQI, Nt.BstNBI, and Nt.CviPII. In embodiments, the endonuclease is Nb.BbvCI or Nt.BsmAI. In embodiments, the endonuclease is Nb.BbvCI. In embodiments, the endonuclease is Nt.BsmAI.

In embodiments, cleaving (e.g., nicking) includes maintaining suitable reaction conditions to permit efficient cleavage (e.g., buffer, pH, temperature conditions). In embodiments, cleaving is performed at about 20° C. to about 60° C. In embodiments, cleavage is performed at about 20° C. to about 30° C., about 30° C. to about 40° C., about 40° C. to about 50° C., or about 50° C. to about 60° C. In embodiments, cleavage is performed at about 20° C., about 25° C., about 30° C., about 35° C., about 37° C., about 40° C., about 42° C., about 45° C., about 48° C., about 50° C., about 55° C., or about 60° C. In embodiments, cleavage is performed at less than 20° C. In embodiments, cleavage is performed at greater than 60° C.

In embodiments, cleavage (e.g., nicking) is performed for about 5 seconds (sec) to about 24 hours (hrs). In embodiments, cleavage is performed for about 5 sec to about 30 sec, about 30 sec to about 60 sec, about 1 minute (min) to about 5 min, about 5 min to about 15 min, about 15 min to about 30 min, about 30 min to about 60 min, about 1 hr to about 4 hrs, about 4 hrs to about 12 hrs, or about 12 hrs to about 24 hrs. In embodiments, cleavage is performed for about 5 sec, 15 sec, 30 sec, 45 sec, 1 min, 2 min, 3 min, 4 min, 5 min, 6 min, 7 min, 8 min, 9 min, 10 min, 11 min, 12 min, 13 min, 14 min, or about 15 min. In embodiments, cleavage is performed for about 20 min, 25 min, 30 min, 35 min, 40 min, 45 min, 50 min, 55 min, or about 1 hr. In embodiments, cleavage is performed for about 2 hrs, 3 hrs, 4 hrs, 5 hrs, 6 hrs, 7 hrs, 8 hrs, 9 hrs, 10 hrs, 11 hrs, or about 12 hrs. In embodiments, cleavage is performed for about 14 hrs, 16 hrs, 18 hrs, 20 hrs, 22 hrs, or about 24 hrs.

In embodiments, cleavage (e.g., nicking) is performed with about 1 unit (U) to about 50 U of endonuclease. The term “unit (U)” or “enzyme unit (U)” is used in accordance with its plain and ordinary meaning, and refers to the amount of the enzyme that catalyzes the conversion of one micromole of substrate per minute under the specified conditions of a given assay. In embodiments, cleavage is performed with about 1 U to about 5 U of endonuclease. In embodiments, cleavage is performed with about 5 U to about 10 U of endonuclease. In embodiments, cleavage is performed with about 10 U to about 15 U of endonuclease. In embodiments, cleavage is performed with about 15 U to about 20 U of endonuclease. In embodiments, cleavage is performed with about 20 U to about 25 U of endonuclease. In embodiments, cleavage is performed with about 25 U to about 35 U of endonuclease. In embodiments, cleavage is performed with about 35 U to about 50 U of endonuclease. In embodiments, cleavage is performed with about 1, 2, 3, 4, 5, 6, 7, 8, 9, 10, 11, 12, 13, 14, 15, 16, 17, 18, 19, 20, 21, 22, 23, 24, 25, 30, 35, 40, 45 or 50 U of endonuclease. In embodiments, cleavage is performed with less than about 1 U of endonuclease. In embodiments, cleavage is performed with greater than about 50 U of endonuclease.

In embodiments, the method further includes hybridizing an oligonucleotide primer to the third extended oligonucleotide, wherein the oligonucleotide primer includes, from 5′ to 3′, a first sequence complementary to the first primer binding sequence and a second sequence complementary to the complement of the second primer binding sequence, extending the second sequence along the third extended oligonucleotide to generate a complementary sequence, and ligating the complementary sequence to the first sequence of the oligonucleotide primer to form a circular oligonucleotide including the complement of the first barcode sequence, the second barcode sequence, and the third barcode sequence. In embodiments, the method further includes amplifying the circular oligonucleotide by extending an amplification primer hybridized to the circular oligonucleotide with a strand-displacing polymerase, wherein the amplification primer extension generates an extension product including multiple complements of the circular oligonucleotide. In embodiments, the method further includes sequencing the circular oligonucleotide. In embodiments, the method further includes sequencing the extension product.

In as aspect is provided a method of forming a circular oligonucleotide including two barcode sequences. In embodiments, the method includes: a) contacting a first biomolecule with a first proximity probe, wherein the first proximity probe includes a first oligonucleotide including, from 5′ to 3′, a first primer binding sequence, a first barcode sequence, and a first probe sequence; b) contacting a second biomolecule with a second proximity probe, wherein the second proximity probe includes a second oligonucleotide including, from 5′ to 3′, a cleavable site, a second primer binding sequence, a second barcode sequence, and a second probe sequence; c) hybridizing the first probe sequence of the first oligonucleotide to the second probe sequence of the second oligonucleotide, and extending the first probe sequence with a polymerase to form an extended oligonucleotide including, from 5′ to 3′, the first primer binding sequence, the first barcode sequence, the first probe sequence, a complement of the second barcode sequence, and a complement of the second primer binding sequence; d) cleaving the cleavable site and removing the second oligonucleotide; and e) hybridizing an oligonucleotide primer to the extended oligonucleotide, wherein the oligonucleotide primer includes, from 5′ to 3′, a first sequence complementary to the first primer binding sequence and a second sequence complementary to the complement of the second primer binding sequence, and extending the second sequence along the extended oligonucleotide to generate a complementary sequence, and ligating the complementary sequence to first sequence of the oligonucleotide primer to form a circular oligonucleotide including the complement of the first barcode sequence and the second barcode sequence.

In as aspect is provided a method of forming a circular oligonucleotide including three barcode sequences, the method including: a) contacting a first biomolecule with a first proximity probe, wherein the first proximity probe includes a first oligonucleotide including, from 5′ to 3′, a first primer binding sequence, a first barcode sequence, and a first probe sequence; b) contacting a second biomolecule with a second proximity probe, wherein the second proximity probe includes a second oligonucleotide including, from 5′ to 3′, a first cleavable site, a second primer binding sequence, a second cleavable site, a second probe sequence, a second barcode sequence, and a third probe sequence; c) contacting a third biomolecule with a third proximity probe, wherein the third proximity probe includes a third oligonucleotide including, from 5′ to 3′, a first cleavable site, the second primer binding sequence, a second cleavable site, a fourth probe sequence, a third barcode sequence, and a fifth probe sequence; d) hybridizing the first probe sequence of the first oligonucleotide to the third probe sequence of the second oligonucleotide, and extending the first probe sequence with a polymerase to form a first extended oligonucleotide including the first primer binding sequence, the first barcode sequence, the first probe sequence, a complement of the second barcode sequence, a complement of the second probe sequence, the second cleavable site, and a complement of the second primer binding sequence; e) cleaving the first cleavable site of the second oligonucleotide, cleaving the second cleavable site of the first extended oligonucleotide, and removing the second oligonucleotide; and f) hybridizing the complement of the second probe sequence of the first extended oligonucleotide to the fifth probe sequence of the third oligonucleotide and extending the complement of the second probe sequence with a polymerase to form a second extended oligonucleotide including the first primer binding sequence, the first barcode sequence, the first probe sequence, the complement of the second barcode sequence, the complement of the second probe sequence, a complement of the third barcode sequence, a complement of the fourth probe sequence, the second cleavable site, and the complement of the second primer binding sequence; g) cleaving the first cleavable sites and removing the third oligonucleotide; and h) hybridizing an oligonucleotide primer to the second extended oligonucleotide, wherein the oligonucleotide primer includes, from 5′ to 3′, a first sequence complementary to the first primer binding sequence and a second sequence complementary to the complement of the second primer binding sequence, and extending the second sequence along the extended oligonucleotide to generate a complementary sequence, and ligating the complementary sequence to first sequence of the oligonucleotide primer to form a circular oligonucleotide including the complement of the first barcode sequence, the second barcode sequence, and the third barcode sequence.

In embodiments, the oligonucleotide (e.g., probe oligonucleotide) includes more than one cleavable site (e.g., a cleavable site at or near the 5′ end of the oligonucleotide or within the linker, and a cleavable site between the 5′ and 3′ end of the oligonucleotide). In embodiments, the oligonucleotide (e.g., probe oligonucleotide) includes a first cleavable site and a second cleavable site, wherein the first and the second cleavable site are separated by about 10, 20, 30, 40, or 50 nucleotides.

In embodiments, cleaving the one or more cleavable sites include orthogonal cleaving methods. In embodiments, the cleavable site includes a sequence that is specifically recognized by a restriction enzyme (e.g., an endonuclease). In embodiments, the restriction endonuclease is BglII. In embodiments, the restriction enzyme is an enzyme described in Table 1. In embodiments, the restriction enzyme recognition sequence included in the cleavable site is selected to be a “rare-cutting” restriction enzyme recognition sequence, e.g., a restriction enzyme that cuts with low frequency in any given genome. For example, Nod is a rare cutter with an eight-base recognition site, which will occur on average about once every 65,000 base pairs in a genome (assuming an average frequency of each type of canonical base of ¼). Other rare-cutting enzymes are known in the art and commercially available, including AbsI, AscI, BbvCI, CciNI, FseI, MreI, PaIAI, RigI, SdaI, and SgsI.

In embodiments, the cleavable site includes one or more deoxyuracil nucleobases (dUs). Any suitable enzymatic, chemical, or photochemical cleavage reaction may be used to cleave the cleavable site. The cleavage reaction may result in removal of a part or the whole of the strand being cleaved. Suitable cleavage means include, for example, restriction enzyme digestion, in which case the cleavable site is an appropriate restriction site for the enzyme which directs cleavage of one or both strands of a duplex template; RNase digestion or chemical cleavage of a bond between a deoxyribonucleotide and a ribonucleotide, in which case the cleavable site may include one or more ribonucleotides; chemical reduction of a disulfide linkage with a reducing agent (e.g., THPP or TCEP), in which case the cleavable site should include an appropriate disulfide linkage; chemical cleavage of a diol linkage with periodate, in which case the cleavable site should include a diol linkage; generation of an abasic site and subsequent hydrolysis, etc. In embodiments, the cleavable site is included in the surface immobilized primer (e.g., within the polynucleotide sequence of the primer). In embodiments, cleavage may be accomplished by using a modified nucleotide as the cleavable site (e.g., uracil, 8oxoG, 5-mC, 5-hmC) that is removed or nicked via a corresponding DNA glycosylase, endonuclease, or combination thereof.

In embodiments, the method includes circularizing and ligating the complementary sequence (e.g., the sequence generated by extending the 3′ end of the oligonucleotide primer which is complementary to the first extended probe oligonucleotide, for example) to the 5′ end of the oligonucleotide primer (e.g., the 5′ end of the extended oligonucleotide primer). In embodiments, the ligation includes enzymatic ligation. In embodiments, the two ends of the extended oligonucleotide primer are ligated directly together. In embodiments, the two ends of the extended oligonucleotide primer are ligated together with the aid of a bridging oligonucleotide (sometimes referred to as a splint oligonucleotide) that is complementary with the two ends of the extended oligonucleotide primer. In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR™ ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, ligating includes chemical ligation (e.g., enzyme-free, click-mediated ligation). In embodiments, the oligonucleotide primer includes a first bioconjugate reactive moiety capable of bonding upon contact with a second (complementary) bioconjugate reactive moiety.

The oligonucleotide primer is similar to a padlock probe, however with an important distinction. Typically, padlock probes hybridize to adjacent sequences and are then ligated together to form a circular oligonucleotide. The oligonucleotide primers hybridize to sequences adjacent to the target nucleic acid sequence resulting in a gap (e.g., a gap spanning the length of the target nucleic acid sequence). Padlock probes are specialized ligation probes, examples of which are known in the art, see for example Nilsson M, et al. Science. 1994; 265(5181):2085-2088), and has been applied to detect transcribed RNA in cells, see for example Christian A T, et al. Proc Natl Acad Sci USA. 2001; 98(25):14238-14243, both of which are incorporated herein by reference in their entireties. The construction of the oligonucleotide primer allows for selective targeting, enabling detection of specific targets within the cell. In embodiments, the oligonucleotide primer includes at least one target-specific region. In embodiments, the oligonucleotide primer includes two target-specific regions. In embodiments, the oligonucleotide primer includes at least one flanking-target region (i.e., an oligonucleotide sequence that flanks the region of interest). In embodiments, the oligonucleotide primer includes two flanking-target regions. A target-specific region is a single stranded polynucleotide that is at least 50% complementary, at least 75% complementary, at least 85% complementary, at least 90% complementary, at least 95% complementary, at least 98%, at least 99% complementary, or 100% complementary to a portion of a nucleic acid molecule that includes a target sequence (e.g., a gene of interest). In embodiments, the target-specific region is capable of hybridizing to at least a portion of the target sequence. In embodiments, the target-specific region is substantially non-complementary to other target sequences present in the sample.

In embodiments, the oligonucleotide primer (i.e., the circularizable oligonucleotide) includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the circularizable oligonucleotide includes one or more LNA nucleotides. In embodiments, the sequence complementary to the first hybridization sequence and/or the second sequence complementary to the second hybridization sequence of the circularizable oligonucleotide includes one or more LNA nucleotides.

In embodiments, the circularizable probe (e.g., the circularizable oligonucleotide) comprises a 5′ end and a 3′ end, wherein a first region at the 5′ end is complementary to a first sequence of a target polynucleotide, and wherein a second region at the 3′ end is complementary to a second sequence of the target polynucleotide. In embodiments, the first sequence and the second sequence of the target polynucleotide are adjacent to each other. In embodiments, the first sequence and the second sequence of the target polynucleotide are separated by 1 or more nucleotides. In embodiments, the first sequence and the second sequence of the target polynucleotide are separated by 1, 5, 10, 20, 30, 40, 50, 75, 100, or more nucleotides. In embodiments, the first sequence and the second sequence of the target polynucleotide flank a target sequence. In embodiments, the target sequence is a barcode sequence.

In embodiments, the circularizable oligonucleotide includes a primer binding sequence. In embodiments, the circularizable oligonucleotide includes at least one primer binding sequence. In embodiments, the circularizable oligonucleotide includes at least two primer binding sequences. In embodiments, the circularizable oligonucleotide includes a primer binding sequence from a known set of primer binding sequences. In embodiments, the circularizable oligonucleotide includes at least two primer binding sequences from a known set of primer binding sequences. In embodiments, the circularizable oligonucleotide includes up to 50 different primer binding sequences from a known set of primer binding sequences. In embodiments, the circularizable oligonucleotide includes up to 10 different primer binding sequences from a known set of primer binding sequences. In embodiments, the circularizable oligonucleotide includes up to 5 different primer binding sequences from a known set of primer binding sequences. In embodiments, the circularizable oligonucleotide includes two or more sequencing primer binding sequences from a known set of sequencing primer binding sequences. In embodiments, the circularizable oligonucleotide includes 1, 2, 3, 4, 5, 6, 7, 8, 9, or 10 primer binding sequences from a known set of primer binding sequences. In embodiments, the circularizable oligonucleotide includes two or more different primer binding sequences from a known set of primer binding sequences. In embodiments, the circularizable oligonucleotide includes 1, 2, 3, 4, 5, 6, 7, 8, 9, or 10 different primer binding sequences from a known set of primer binding sequences. In embodiments, the circularizable oligonucleotide includes 2 to 5 primer binding sequences from a known set of primer binding sequences. In embodiments, the circularizable oligonucleotide includes 2 to 5 different primer binding sequences from a known set of primer binding sequences. In embodiments, the circularizable oligonucleotide includes 2 to 5 sequencing primer binding sequences from a known set of sequencing primer binding sequences. In embodiments, the circularizable oligonucleotide includes 2 to 5 different sequencing primer binding sequences from a known set of sequencing primer binding sequences. In embodiments, the circularizable oligonucleotide includes at least two different primer binding sequences. In embodiments, the circularizable oligonucleotide includes two different sequencing primer binding sequences.

In embodiments, the circularizable oligonucleotide includes one or more ribonucleotides. In embodiments, the circularizable oligonucleotide includes at least one ribonucleotide at or near the ligation site (i.e., any of the 10 nucleotides within 5 nucleotides of the ligation site, wherein the ligation site includes the 5′ or 3′ end of the circularizable oligonucleotide). In embodiments, the circularizable oligonucleotide includes a ribonucleotide at a 3′ terminal and/or 3′ penultimate nucleotide. In embodiments, the circularizable oligonucleotide does not include a ribonucleotide at the 5′ end. In embodiments, the circularizable oligonucleotide does not include more than 4 consecutive ribonucleotides. Additional compositions and methods thereof of circularizable oligonucleotides including ribonucleotides are described in, e.g., U.S. Pat. Pub. No. US 2020/0224244, which is incorporated herein by reference in its entirety.

In embodiments, the oligonucleotide primer is approximately 50 to 200 nucleotides. In embodiments, the oligonucleotide primer has a first domain that is capable of hybridizing to a first target sequence domain, and a second ligation domain, capable of hybridizing to a target nucleic acid sequence-adjacent second sequence domain. In embodiments, following hybridization there is a gap between the first target sequence domain, and the second ligation domain, wherein the gap spans the length of the target nucleic acid sequence.

In embodiments, the oligonucleotide primer includes at least one primer binding sequence. In embodiments, the oligonucleotide primer includes at least two primer binding sequences. In embodiments, the oligonucleotide primer includes an amplification primer binding sequence. In embodiments, the oligonucleotide primer includes a sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, the method further includes amplifying the circular oligonucleotide by extending an amplification primer hybridized to the circular oligonucleotide with a strand-displacing polymerase, wherein the amplification primer extension generates an extension product including multiple complements of the circular oligonucleotide. In embodiments, the method further includes sequencing the extension product.

In embodiments, the amplification primer binding sequence and/or sequencing primer binding sequence includes any one of the sequences (e.g., all or a portion thereof), or complement thereof, as described in Table 2. In embodiments, the amplification primer binding sequence includes any one of the sequences, or complement thereof, of SEQ ID NO:21 to SEQ ID NO:74. In embodiments, the sequencing primer binding sequence includes any one of the sequences, or complement thereof, of SEQ ID NO:21 to SEQ ID NO:74. In embodiments, the amplification primer binding sequence includes any one of the sequences, or complement thereof, of SEQ ID NO: 21, SEQ ID NO:22, SEQ ID NO:23, SEQ ID NO:25, SEQ ID NO:27, SEQ ID NO:48, or SEQ ID NO:53. In embodiments, the sequencing primer binding sequence includes any one of the sequences, or complement thereof, of SEQ ID NO: 21, SEQ ID NO:22, SEQ ID NO:23, SEQ ID NO:25, SEQ ID NO:27, SEQ ID NO:48, or SEQ ID NO:53. In embodiments, the amplification primer binding sequence includes any one of the sequences, or complement thereof, of SEQ ID NO: 27, SEQ ID NO:62, SEQ ID NO:37, SEQ ID NO:48, SEQ ID NO:22, SEQ ID NO:67, or SEQ ID NO:53. In embodiments, the sequencing primer binding sequence includes any one of the sequences, or complement thereof, of SEQ ID NO: 27, SEQ ID NO:62, SEQ ID NO:37, SEQ ID NO:48, SEQ ID NO:22, SEQ ID NO:67, or SEQ ID NO:53.

TABLE 2
Effective primer sequences. It is understood that white space,
line breaks, and text formatting are not indicative of separate
sequences or structural implications. The target polynucleotides
may be amplified using primers with the sequences identified in
this table. In embodiments, one or more of the nucleotides are
LNA nucleotides, e.g., nucleotides at the 5' end, to modulate
the melting temperature.

| Primer | | SEQ ID |
|---|---|---|
| Name | Sequence (5′→3′) | Num. |
| S1 | ACAAAGGCAGCCACG CACTCCTTCCCTGT | SEQ ID |
| | | NO: 21 |
| SP1 | ACACTCTTTCCCTACA C GACGCTCTTCCGATCT | SEQ ID |
| | | NO: 22 |
| S2 | CTCCAGCGAGATGACC CTCACCAACCACT | SEQ ID |
| | | NO: 23 |
| SP2 | GTGACTGGAGTTCAGA CGTGTGCTCTTCCGATCT | SEQ ID |
| | | NO: 24 |
| P5 | AATGATACGGCGACCACCG | SEQ ID |
| | | NO: 25 |
| P7 | CAAGCAGAAGACGGCATACGAGAT | SEQ ID |
| | | NO: 26 |
| M1A | AACGCCAAACCTACGGCTTTACTTCCTGTGGCT | SEQ ID |
| | | NO: 27 |
| M2A | TCTTGAGTCATTCGCAGGGCATGTGCCAGACCT | SEQ ID |
| | | NO: 28 |
| M3A | TCGGCGTTGTCTGCTATCGTTCTTGGCACTCCT | SEQ ID |
| | | NO: 29 |
| M4A | GGAGCAATAACCATAAGGCCGTTGACAAGCCCT | SEQ ID |
| | | NO: 30 |
| M5A | GGCGTATTGCCTTGGTTCTGGCAGCCTCATTGT | SEQ ID |
| | | NO: 31 |
| M1B | CAGCAGAGGGAACGATTTCAACTTCCTGTGGCT | SEQ ID |
| | | NO: 32 |
| M2B | CTACTGCAAGGGTGTCTAGAATGTGCCAGACCT | SEQ ID |
| | | NO: 33 |
| M3B | GACCGACTCGTGAAACGTAATCTTGGCACTCCT | SEQ ID |
| | | NO: 34 |
| M4B | ACACATTCTTTGCGCCCAGAGTTGACAAGCCCT | SEQ ID |
| | | NO: 35 |
| M5B | ATTTCATTCGACACCCGGTCGCAGCCTCATTGT | SEQ ID |
| | | NO: 36 |
| M1A_RC | AGCCACAGGAAGTAAAGCCGTAGGTTTGGCGTT | SEQ ID |
| | | NO: 37 |
| M2A_RC | AGGTCTGGCACATGCCCTGCGAATGACTCAAGA | SEQ ID |
| | | NO: 38 |
| M3A_RC | AGGAGTGCCAAGAACGATAGCAGACAACGCCGA | SEQ ID |
| | | NO: 39 |
| M4A_RC | AGGGCTTGTCAACGGCCTTATGGTTATTGCTCC | SEQ ID |
| | | NO: 40 |
| M5A_RC | ACAATGAGGCTGCCAGAACCAAGGCAATACGCC | SEQ ID |
| | | NO: 41 |
| M1B_RC | AGCCACAGGAAGTTGAAATCGTTCCCTCTGCTG | SEQ ID |
| | | NO: 42 |
| M2B_RC | AGGTCTGGCACATTCTAGACACCCTTGCAGTAG | SEQ ID |
| | | NO: 43 |
| M3B_RC | AGGAGTGCCAAGATTACGTTTCACGAGTCGGTC | SEQ ID |
| | | NO: 44 |
| M4B_RC | AGGGCTTGTCAACTCTGGGCGCAAAGAATGTGT | SEQ ID |
| | | NO: 45 |
| M5B_RC | ACAATGAGGCTGCGACCGGGTGTCGAATGAAAT | SEQ ID |
| | | NO: 46 |
| M6A | TGTTGCATCTCCACCCGGATTGAGCCTTCAGCT | SEQ ID |
| | | NO: 47 |
| M7A | CACAACGGGAGCTGTGGAATTGGTTCACCTGGT | SEQ ID |
| | | NO: 48 |
| M8A | TGGACTAAGACTCGTCCTCCAGCGGACCTAAGT | SEQ ID |
| | | NO: 49 |
| M9A | GTATGATGGTGTTGCGGCTTCTCGCTTAACGCT | SEQ ID |
| | | NO: 50 |
| M10A | TCTGAGTGCCAGTGACTTCACGCATTCGCTTGT | SEQ ID |
| | | NO: 51 |
| M11A | TACGACACACTCGGGCTCTATGGGCTTCATGGT | SEQ ID |
| | | NO: 52 |
| M12A | GTTTGAGTGAAGGCGGTCCAACCCTTAGTGCGT | SEQ ID |
| | | NO: 53 |
| M6B | CTATAAGTTTGTCGTGCCCGTGAGCCTTCAGCT | SEQ ID |
| | | NO: 54 |
| M7B | GGAGTGACACTGACTACGTTTGGTTCACCTGGT | SEQ ID |
| | | NO: 55 |
| M8B | GTCAACGCCCTAGCAGACATAGCGGACCTAAGT | SEQ ID |
| | | NO: 56 |
| M9B | CCAGAACCTATTGAGCCTGACTCGCTTAACGCT | SEQ ID |
| | | NO: 57 |
| M10B | AGGTGTTCGTACAATGAGGCCGCATTCGCTTGT | SEQ ID |
| | | NO: 58 |
| M11B | TGGTCAAGGGCAACTAATCCTGGGCTTCATGGT | SEQ ID |
| | | NO: 59 |
| M12B | ACAATTACCCGTTTACCGGCACCCTTAGTGCGT | SEQ ID |
| | | NO: 60 |
| M6A_RC | AGCTGAAGGCTCAATCCGGGTGGAGATGCAACA | SEQ ID NO: |
| | | 61 |
| M7A_RC | ACCAGGTGAACCAATTCCACAGCTCCCGTTGTG | SEQ ID NO: |
| | | 62 |
| M8A_RC | ACTTAGGTCCGCTGGAGGACGAGTCTTAGTCCA | SEQ ID NO: |
| | | 63 |
| M9A_RC | AGCGTTAAGCGAGAAGCCGCAACACCATCATAC | SEQ ID NO: |
| | | 64 |
| M10A_RC | ACAAGCGAATGCGTGAAGTCACTGGCACTCAGA | SEQ ID NO: |
| | | 65 |
| M11A_RC | ACCATGAAGCCCATAGAGCCCGAGTGTGTCGTA | SEQ ID NO: |
| | | 66 |
| M12A_RC | ACGCACTAAGGGTTGGACCGCCTTCACTCAAAC | SEQ ID NO: |
| | | 67 |
| M6B_RC | AGCTGAAGGCTCACGGGCACGACAAACTTATAG | SEQ ID NO: |
| | | 68 |
| M7B_RC | ACCAGGTGAACCAAACGTAGTCAGTGTCACTCC | SEQ ID NO: |
| | | 69 |
| M8B_RC | ACTTAGGTCCGCTATGTCTGCTAGGGCGTTGAC | SEQ ID NO: |
| | | 70 |
| M9B_RC | AGCGTTAAGCGAGTCAGGCTCAATAGGTTCTGG | SEQ ID NO: |
| | | 71 |
| M10B_RC | ACAAGCGAATGCGGCCTCATTGTACGAACACCT | SEQ ID NO: |
| | | 72 |
| M11B_RC | ACCATGAAGCCCAGGATTAGTTGCCCTTGACCA | SEQ ID NO: |
| | | 73 |
| M12B_RC | ACGCACTAAGGGTGCCGGTAAACGGGTAATTGT | SEQ ID NO: |
| | | 74 |

In embodiments, the method further includes sequencing the circular oligonucleotide. In embodiments, the method further includes sequencing the one or more barcodes, or complements thereof, of the circular oligonucleotide. In embodiments, the method further includes sequencing the two or more barcodes, or complements thereof, of the circular oligonucleotide. In embodiments, the method further includes sequencing the three or more barcodes, or complements thereof, of the circular oligonucleotide. Sequencing may be performed in situ or in embodiments, the circular oligonucleotide is isolated and sequenced on a separate instrument.

In embodiments, the circular oligonucleotide that is about 100 to about 1000 nucleotides in length, about 100 to about 300 nucleotides in length, about 300 to about 500 nucleotides in length, or about 500 to about 1000 nucleotides in length. In embodiments, the circular oligonucleotide is about 300 to about 600 nucleotides in length. In embodiments, the circular oligonucleotide is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides in length. In embodiments, the circular oligonucleotide molecule is about 100-1000 nucleotides in length. In embodiments, the circular oligonucleotide molecule is about 100-300 nucleotides in length. In embodiments, the circular oligonucleotide molecule is about 300-500 nucleotides in length. In embodiments, the circular oligonucleotide molecule is about 500-1000 nucleotides in length. In embodiments, the circular oligonucleotide molecule is about 100 nucleotides. In embodiments, the circular oligonucleotide molecule is about 300 nucleotides. In embodiments, the circular oligonucleotide molecule is about 500 nucleotides. In embodiments, the circular oligonucleotide molecule is about 1000 nucleotides. Circular oligonucleotides may be conveniently isolated by a conventional purification column, digestion of non-circular DNA by one or more appropriate exonucleases, or both.

In embodiments, the first biomolecule, the second biomolecule, and the third biomolecule are different biomolecules (e.g., the first, second, and third biomolecule are on different proteins). In embodiments, the first biomolecule, the second biomolecule, and the third biomolecule are the same biomolecules (e.g., the first, second, and third biomolecule are on the same protein). In embodiments, the first biomolecule and the second biomolecule are different biomolecules (e.g., the first and second biomolecules are on different proteins). In embodiments, the first biomolecule and the second biomolecule are the same biomolecules (e.g., the first and second biomolecules are on the same protein). In embodiments, the first biomolecule and the third biomolecule are different biomolecules (e.g., the first and third biomolecules are on different proteins). In embodiments, the first biomolecule and the third biomolecule are the same biomolecules (e.g., the first and third biomolecules are on the same protein). In embodiments, the second biomolecule and the third biomolecule are different biomolecules (e.g., the second and third biomolecules are on different proteins). In embodiments, the second biomolecule and the third biomolecule are the same biomolecules (e.g., the second and third biomolecules are on the same protein). In embodiments, all of the biomolecules are different biomolecules. In embodiments, all of the biomolecules are the same biomolecule. In embodiments, a portion of the biomolecules are different biomolecules. In embodiments, a portion of the biomolecules are the same biomolecule.

In embodiments, the biomolecule is a nucleic acid molecule. In embodiments, the biomolecule is a lipid, carbohydrate, peptide, protein, or antigen binding fragment. In embodiments, the biomolecule is a glycoprotein, lipoprotein, or phosphoprotein.

In embodiments, the biomolecule is in a cell. In embodiments, the biomolecule is on a cell. In embodiments, the biomolecule is in a tissue.

In embodiments, the method further includes sequencing each barcode to obtain a multiplexed signal in the cell in situ; demultiplexing the multiplexed signal by comparison with the known set of barcodes; and detecting the plurality of targets (e.g., the plurality of target biomolecules) by identifying the associated barcodes detected in the cell. In embodiments, demultiplexing the multiplexed signal includes a linear decomposition of the multiplexed signal. Any of a variety of techniques may be employed for decomposition of the multiplexed signal. Examples include, but are not limited to, Zimmerman et al. Chapter 5: Clearing Up the Signal: Spectral Imaging and Linear Unmixing in Fluorescence Microscopy; Confocal Microscopy: Methods and Protocols, Methods in Molecular Biology, vol. 1075 (2014); Shirawaka H. et al.; Biophysical Journal Volume 86, Issue 3, March 2004, Pages 1739-1752; and S. Schlachter, et al, Opt. Express 17, 22747-22760 (2009); the content of each of which is incorporated herein by reference in its entirety. In embodiments, multiplexed signal includes overlap of a first signal and a second signal and is computationally resolved, for example, by imaging software. In embodiments, more than one analyte type (e.g., nucleic acids and proteins) from a biological sample can be detected (e.g., simultaneously or sequentially) using any appropriate multiplexing technique.

In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. An oligonucleotide barcode is at least about 1, 2, 3, 4, 5, 6, 7, 8, 9, 10, 15, 20, 25, 30, 35, 40, 45, 50 or more nucleotides in length. An oligonucleotide barcode can be at most about 300, 200, 100, 90, 80, 70, 60, 50, 40, 30, 20, 15, 12, 10, 9, 8, 7, 6, 5, 4 or fewer or more nucleotides in length. In embodiments, an oligonucleotide barcode includes between about 5 to about 8, about 5 to about 10, about 5 to about 15, about 5 to about 20, about 10 to about 150 nucleotides. In embodiments, an oligonucleotide barcode includes between 5 to 8, 5 to 10, 5 to 15, 5 to 20, 10 to 150 nucleotides. In embodiments, an oligonucleotide barcode is 10 nucleotides. An oligonucleotide barcode may include a unique sequence (e.g., a barcode sequence) that gives the oligonucleotide barcode its identifying functionality. The unique sequence may be random or non-random. Attachment of the barcode sequence (via bind of a proximity probe conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the oligonucleotide barcode consists only of a unique barcode sequence. In embodiments, the 5′ end of a barcoded oligonucleotide is phosphorylated. In embodiments, the oligonucleotide barcode is known (i.e., the nucleic sequence is known before sequencing) and is sorted into a basis-set according to their Hamming distance. Oligonucleotide barcodes can be associated with a target of interest by knowing, a priori, the target of interest, such as a gene or protein. In embodiments, the oligonucleotide barcodes further include one or more sequences capable of specifically binding a gene or nucleic acid sequence of interest. For example, in embodiments, the oligonucleotide barcode include a sequence capable of hybridizing to mRNA, e.g., one containing a poly-T sequence (e.g., having several T's in a row, e.g., 4, 5, 6, 7, 8, or more T's).

In embodiments, the oligonucleotide barcode is included as part of an oligonucleotide of longer sequence length, such as a primer or a random sequence (e.g., a random N-mer). In embodiments, the oligonucleotide barcode contains random sequences to increase the mass or size of the oligonucleotide tag. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides.

In embodiments, the oligonucleotide barcode is a nucleic acid molecule which can hybridize specifically to a target (e.g., a nucleic acid of interest). The unique identifier sequence of the barcode can be a nucleic acid sequence which associates the oligonucleotide barcode with the nucleic acid of interest to which it hybridizes.

In embodiments, the oligonucleotide barcode is taken from a “pool” or “set” or “basis-set” of potential oligonucleotide barcode sequences. The set of oligonucleotide barcodes may be selected using any suitable technique, e.g., randomly, or such that the sequences allow for error detection and/or correction, or having a particular feature, such as by being separated by a certain distance (e.g., Hamming distance). In embodiments, the method includes selecting a basis-set of oligonucleotide barcodes having a specified Hamming distance (e.g., a Hamming distance of 10; a Hamming distance of 5). The pool may have any number of potential barcode sequences, e.g., at least 100, at least 300, at least 500, at least 1,000, at least 3,000, at least 5,000, at least 10,000, at least 30,000, at least 50,000, at least 100,000, at least 300,000, at least 500,000, or at least 1,000,000 barcode sequences. In embodiments, a barcode is a degenerate or partially-degenerate sequence, such that one or more nucleotides are selected at random from a set of two or more different nucleotides at one or more positions, with each of the different nucleotides selected at one or more positions represented in a pool of oligonucleotides including the degenerate or partially-degenerate sequence. The number of possible barcodes in a given set of barcodes will vary with the number of degenerate positions, and the number of bases permitted at each such position. For example, a barcode of five nucleotides (consecutive or non-consecutive), in which each position can be any of A, T, G, or C represents 54, or 1024 possible barcodes. In embodiments, certain barcode sequences may be excluded from a pool, such as barcodes in which every position is the same base. In embodiments, there are about, 102, 103 104, 105, 106, 107, 108, 109, or a number or a range between any two of these values, unique nucleotide barcode sequences. In embodiments, there are at least, or at most 102, 103 104, 105, 106, 107, 108, 109 unique barcode sequences. In embodiments, a barcode is about, 1, 2, 3, 4, 5, 10, 15, 20, 25, 30, 35, 40, 45, 50, or a number or a range between any two of these values, nucleotides in length. A barcode can be at least, or at most, 1, 2, 3, 4, 5, 10, 15, 20, 25, 30, 35, 40, 45, 50, 100, or 200 nucleotides in length.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest. The use of oligonucleotide barcodes with unique identifier sequences as described herein allows for simultaneous detection of 2, 3, 4, 5, 6, 7, 8, 9, 10, 20, 30, 40, 50, 60, 70, 80, 90, 100, 200, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000 or more than 10,000 unique targets within a single cell. In contrast to existing in situ detection methods, the methods presented herein have the advantage of virtually limitless numbers of individually detected molecules in parallel and in situ.

In embodiments, the proximity probe is an antibody, an antibody fragment, an affimer, an aptamer, or a nucleic acid. The antibodies used for the protein proximity probes may be polyclonal or monoclonal antibodies, or fragments of antibodies. Further, the antibodies linked to each member of the protein proximity probe pair may have the same binding specificity or differ in their binding specificities. Further contemplated herein is the use of variations of this assay, e.g., that are described in WO2012/104261, which is incorporated herein by reference in its entirety. For example, the probes may each be linked to their respective antibody at the 5′ end, or one probe may be linked at the 5′ end and the other at the 3′ end.

A proximity probe is defined herein as an entity including an analyte-binding domain specific for a biomolecule, and a nucleic acid domain (e.g., a probe oligonucleotide). By “specific for biomolecule” is meant that the biomolecule-binding domain specifically recognizes and binds a particular target biomolecule, i.e., it binds its target biomolecule with higher affinity than it binds to other biomolecules or moieties. In embodiments, the biomolecule-binding domain is an antibody, in particular a monoclonal antibody. Antibody fragments or derivatives of antibodies including the biomolecule-binding domain are also suitable for use as the biomolecule binding domain. Examples of such antibody fragments or derivatives include Fab, Fab′, F(ab′)2 and scFv molecules.

A Fab fragment consists of the antigen-binding domain of an antibody. An individual antibody may be seen to contain two Fab fragments, each consisting of a light chain and its conjoined N-terminal section of the heavy chain. Thus, a Fab fragment contains an entire light chain and the VH and CH1 domains of the heavy chain to which it is bound. Fab fragments may be obtained by digesting an antibody with papain.

F(ab′)2 fragments consist of the two Fab fragments of an antibody, plus the hinge regions of the heavy domains, including the disulfide bonds linking the two heavy chains together. In other words, a F(ab′)2 fragment can be seen as two covalently joined Fab fragments. F(ab′)2 fragments may be obtained by digesting an antibody with pepsin. Reduction of F(ab′)2 fragments yields two Fab′ fragments, which can be seen as Fab fragments containing an additional sulfhydryl group which can be useful for conjugation of the fragment to other molecules. ScFv molecules are synthetic constructs produced by fusing together the variable domains of the light and heavy chains of an antibody. Typically, this fusion is achieved recombinantly, by engineering the antibody gene to produce a fusion protein which includes both the heavy and light chain variable domains. The nucleic acid domain of a proximity probe may be a DNA domain or an RNA domain. Preferably it is a DNA domain. The nucleic acid domains (e.g., probe oligonucleotide) of the proximity probes typically are designed to hybridize to one another, or to one or more common oligonucleotide molecules (e.g., one or more probe sequences in the probe oligonucleotide of one or more proximity probes, to which the probe oligonucleotides of both proximity probes of a pair may hybridize). Accordingly, the probe oligonucleotides must be at least partially single-stranded. In certain embodiments the probe oligonucleotides of the proximity probes are wholly single-stranded. In other embodiments, the probe oligonucleotides of the proximity probes are partially single-stranded, including both a single-stranded part and a double-stranded part.

In embodiments, the first proximity probe and the second proximity probe bind to the same target biomolecule (e.g., an individual protein). In this embodiment, both proximity probes bind the target biomolecule (e.g. protein), but at different epitopes. The epitopes are non-overlapping, so that the binding of one probe in the pair to its epitope does not interfere with or block binding of the other probe in the pair to its epitope. Alternatively, the target biomolecule may be a complex, e.g. a protein complex, in which case one probe in the pair binds one member of the complex and the other probe in the pair binds the other member of the complex. The probes bind the proteins within the complex at sites different to the interaction sites of the proteins (i.e., the sites in the proteins through which they interact with each other).

In embodiments, steps (a)-(c) are performed in situ. In embodiments, steps (d)-(f) are performed in situ. In embodiments, all steps of a method described herein are performed in situ.

In embodiments, following step (f), the method further includes: (g) cleaving the complement of the cleavable site of the second extended oligonucleotide, cleaving the cleavable site of the third oligonucleotide, and removing the third oligonucleotide. In embodiments, the method further includes (h) hybridizing the complement of the fourth probe sequence of the second extended oligonucleotide to a fourth proximity probe including a fourth oligonucleotide, and extending the second extended oligonucleotide with a polymerase to form a third extended oligonucleotide, wherein the fourth proximity probe is contacted to a fourth biomolecule, and wherein the fourth oligonucleotide includes a fourth barcode sequence. In embodiments, the method further includes cleaving a cleavable site on the third extended oligonucleotide and repeating steps (g)-(h) for one or more additional proximity probes include an oligonucleotide including a barcode sequence.

In embodiments, the first oligonucleotide is attached to the first proximity probe via a linker, and the second oligonucleotide is attached to the second proximity probe via a linker. In embodiments, the second oligonucleotide is attached to the second proximity probe via a cleavable linker. In embodiments, the third oligonucleotide is attached to the third proximity probe via a cleavable linker. In embodiments, the cleavable linker includes one or more cleavable sites. In embodiments, the cleavable linker includes a polynucleotide or a polypeptide sequence. In embodiments, the cleavable linker includes a cleavable site as described herein.

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell, a fungal cell, a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the cell is a stem cell, an immune cell, a cancer cell, a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell.

In embodiments, the cell is a prokaryotic cell. In embodiments, the cell is a bacterial cell. In embodiments, the bacterial cell is a Bacteroides, Clostridium, Faecalibacterium, Eubacterium, Ruminococcus, Peptococcus, Peptostreptococcus, or Bifidobacterium cell. In embodiments, the bacterial cell is a Bacteroides fragilis, Bacteroides melaninogenicus, Bacteroides oralis, Enterococcus faecalis, Escherichia coli, Enterobacter sp., Klebsiella sp., Bifidobacterium bifidum, Staphylococcus aureus, Lactobacillus, Clostridium perfringens, Proteus mirabilis, Clostridium tetani, Clostridium septicum, Pseudomonas aeruginosa, Salmonella enterica, Faecalibacterium prausnitzii, Peptostreptococcus sp., or Peptococcus sp. cell. In embodiments, the cell is a fungal cell. In embodiments, the fungal cell is a Candida, Saccharomyces, Aspergillus, Penicillium, Rhodotorula, Trametes, Pleospora, Sclerotinia, Bullera, or a Galactomyces cell. In embodiments, the cell is a viral-host cell. A “viral-host cell” is used in accordance with its ordinary meaning in virology and refers to a cell that is infected with a viral genome (e.g., viral DNA or viral RNA). The cell, prior to infection with a viral genome, can be any cell that is susceptible to viral entry. In embodiments, the viral-host cell is a lytic viral-host cell. In embodiments, the viral-host cell is capable of producing viral protein. In embodiments, the viral-host cell is a lysogenic viral-host cell. In embodiments, the cell is a viral-host cell including a viral nucleic acid sequence, wherein the viral nucleic acid sequence is from a Hepadnaviridae, Adenoviridae, Herpesviridae, Poxviridae, Parvoviridae, Reoviridae, Coronaviridae, Retroviridae virus.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. Examples of such cells include, but are not limited to, cell lines derived from hematopoietic cells, and from the following cell lines: Colo205, CCRF-CEM, HL-60, K562, MOLT-4, RPMI-8226, SR, HOP-92, NCI-H322M, and MALME-3M. Non-limiting examples of adherent cells include DU145 (prostate cancer) cells, H295R (adrenocortical cancer) cells, HeLa (cervical cancer) cells, KBM-7 (chronic myelogenous leukemia) cells, LNCaP (prostate cancer) cells, MCF-7 (breast cancer) cells, MDA-MB-468 (breast cancer) cells, PC3 (prostate cancer) cells, SaOS-2 (bone cancer) cells, SH-SY5Y (neuroblastoma, cloned from a myeloma) cells, T-47D (breast cancer) cells, THP-1 (acute myeloid leukemia) cells, U87 (glioblastoma) cells, National Cancer Institute's 60 cancer cell line panel (NCI60), vero (African green monkey Chlorocebus kidney epithelial cell line) cells, MC3T3 (embryonic calvarium) cells, GH3 (pituitary tumor) cells, PC12 (pheochromocytoma) cells, dog MDCK kidney epithelial cells, Xenopus A6 kidney epithelial cells, zebrafish AB9 cells, and Sf9 insect epithelial cells. In embodiments, the cell is a neuronal cell, an endothelial cell, epithelial cell, germ cell, plasma cell, a muscle cell, peripheral blood mononuclear cell (PBMC), a myocardial cell, or a retina cell.

In embodiments, the cell is bound to a known antigen. In embodiments, the cell is a cell that selectively binds to a desired target, wherein the target is an antibody, or antigen binding fragment, an aptamer, affimer, non-immunoglobulin scaffold, small molecule, or genetic modifying agent. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a T cell, an NK cell, or a B cell. In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In embodiments, the cell is a cancer cell. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. In embodiments, the cancer cell includes a HER2 gene (see for example FIG. 6). In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma. In embodiments, the cancer-associated gene is a nucleic acid sequence identified within The Cancer Genome Atlas Program, accessible at www.cancer.gov/tcga.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface (e.g., a microplate). In embodiments, the cell is permeabilized and immobilized within a well of the microplate. In embodiments, the cell is immobilized to a solid support surface (e.g., a well or a slide). In embodiments, the surface includes a patterned surface (e.g., suitable for immobilization of a plurality of cells in an ordered pattern. In embodiments, a plurality of cells is immobilized in wells of a microplate that have a mean or median separation from one another of about 10-20 μm. In embodiments, a plurality of cells is immobilized in wells of a microplate that have a mean or median separation from one another of about 10-20; 10-50; or 100 μm. In embodiments, a plurality of cells is arrayed on a substrate.

In embodiments, the cell is attached to the substrate via a bioconjugate reactive linker. In embodiments, the cell is attached to the substrate via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. Substrates may be prepared for selective capture of particular cells. For example, a substrate containing a plurality of bioconjugate reactive moieties or a plurality of specific binding reagents, optionally in an ordered pattern, contacts a plurality of cells. Only cells containing complementary bioconjugate reactive moieties or complementary specific binding reagents are capable of reacting, and thus adhering, to the substrate. In embodiments, the cell is immobilized to a substrate. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.

In embodiments, the methods are performed in situ on isolated cells or in tissue sections (alternatively referred to as a sample) that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the cell is cleared (e.g., digested) of proteins, lipids, or proteins and lipids. In embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypin, proteases (e.g., pepsin and/or proteinase K)). In embodiments, the biological sample can be permeabilized by contacting the sample with a permeabilization solution. In some embodiments, the biological sample is permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 w/v %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some embodiments, the biological sample can be permeabilized by exposing the sample (e.g., for about 5 minutes to about 1 hour, about 5 minutes to about 40 minutes, about 5 minutes to about 30 minutes, about 5 minutes to about 20 minutes, or about 5 minutes to about 10 minutes) to about 1.0 w/v % to about 14.0 w/v % (e.g., about 2.0 w/v % to about 14.0 w/v %, about 2.0 w/v % to about 12.0 w/v %, about 2.0 w/v % to about 10.0 w/v %, about 4.0 w/v % to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v % to about 14.0 w/v %) SDS and/or N-lauroylsarcosine salt solution and/or proteinase K (e.g., at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

In embodiments, the cell is exposed to paraformaldehyde (i.e., by contacting the cell with paraformaldehyde). In embodiments, the cell is exposed to glutaraldehyde (i.e., by contacting the cell with glutaraldehyde). Any suitable permeabilization and fixation technologies can be used for making the cell available for the detection methods provided herein. In embodiments the method includes affixing single cells or tissues to a transparent substrate. Exemplary tissue includes those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv©, FineFIX®, RCL2/KINFix, UMFIX, Glyo-Fixx®, Histochoice®, or PAXgene®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM). In embodiments, the sample can be a biological sample selected from the group consisting of a freshly isolated sample, a fixed sample, a frozen sample, an embedded sample, a processed sample, or a combination thereof.

In embodiments the cell is lysed to release nucleic acid or other materials from the cells. For example, the cells may be lysed using reagents (e.g., a surfactant such as Triton-X or SDS, an enzyme such as lysozyme, lysostaphin, zymolase, cellulase, mutanolysin, glycanases, proteases, mannase, proteinase K, etc.) or a physical lysing mechanism a physical condition (e.g., ultrasound, ultraviolet light, mechanical agitation, etc.). The cells may release, for instance, DNA, RNA, mRNA, proteins, or enzymes. The cells may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally occurring sample (e.g., pond water, soil, etc.), or the like. In some cases, the cells may be dissociated from tissue. In embodiments, the method does not include dissociating the cell from the tissue or the cellular microenvironment. In embodiments, the method does not include lysing the cell.

In embodiments, a permeabilization solution can contain additional reagents or a biological sample may be treated with additional reagents in order to optimize biological sample permeabilization. In some embodiments, an additional reagent is an RNA protectant. As used herein, the term “RNA protectant” typically refers to a reagent that protects RNA from RNA nucleases (e.g., RNases). Any appropriate RNA protectant that protects RNA from degradation can be used. A non-limiting example of an RNA protectant includes organic solvents (e.g., at least 60%, 65%, 70%, 75%, 80%, 85%, 90%, or 95% v/v organic solvent), which includes ethanol, methanol, propan-2-ol, acetone, trichloroacetic acid, propanol, polyethylene glycol, acetic acid, or a combination thereof. In embodiments, the RNA protectant includes ethanol, methanol and/or propan-2-ol, or a combination thereof. In embodiments, the RNA protectant includes RNAlater ICE (ThermoFisher Scientific). In embodiments, the RNA protectant includes a salt. The salt may include ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate and zinc sulfate. In some embodiments, the biological sample is treated with one or more RNA protectants before, contemporaneously with, or after permeabilization.

In embodiments, the method further includes subjecting the cell to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety.

In embodiments, the method does not include subjecting the cell to expansion microscopy. Typically, expansion microscopy techniques utilize a swellable polymer or hydrogel (e.g., a synthetic matrix-forming material) which can significantly slow diffusion of enzymes and nucleotides. Matrix forming materials (e.g., a synthetic matrix) include polyacrylamide, cellulose, alginate, polyamide, cross-linked agarose, cross-linked dextran or cross-linked polyethylene glycol. The matrix forming materials can form a matrix by polymerization and/or crosslinking of the matrix forming materials using methods specific for the matrix forming materials and methods, reagents and conditions known to those of skill in the art. Additionally, expansion microscopy techniques may render the temperature of the cell sample difficult to modulate in a uniform, controlled manner. Modulating temperature provides a useful parameter to optimize amplification and sequencing methods.

In embodiments the biomolecule (otherwise referred to herein as a target) is an RNA transcript. In embodiments the target is a single stranded RNA nucleic acid sequence. In embodiments, the target is an RNA nucleic acid sequence or a DNA nucleic acid sequence (e.g., cDNA). In embodiments, the target is a cDNA target nucleic acid sequence and before step i), the RNA nucleic acid sequence is reverse transcribed to generate the cDNA target nucleic acid sequence. In embodiments, the target is genomic DNA (gDNA), mitochondrial DNA, chloroplast DNA, episomal DNA, viral DNA, or copy DNA (cDNA). In embodiments, the target is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target is a cancer-associated gene. In embodiments, to minimize amplification errors or bias, the target is not reverse transcribed to generate cDNA.

In embodiments, the target is an RNA nucleic acid sequence or DNA nucleic acid sequence. In embodiments, the target is an RNA nucleic acid sequence or DNA nucleic acid sequence from the same cell. In embodiments, the target is an RNA nucleic acid sequence. In embodiments, the RNA nucleic acid sequence is stabilized using known techniques in the art. For example, RNA degradation by RNase should be minimized using commercially available solutions, e.g., RNA Later®, RNA Lysis Buffer, or Keratinocyte serum-free medium). In embodiments, the target is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA). In embodiments, the target is mRNA, tRNA (transfer RNA), rRNA (ribosomal RNA), or noncoding RNA (such as lncRNA (long noncoding RNA)). In embodiments, the targets are on different regions of the same RNA nucleic acid sequence. In embodiments, the targets are cDNA target nucleic acid sequences and before step i), the RNA nucleic acid sequences are reverse transcribed to generate the cDNA target nucleic acid sequences. In embodiments, the targets are not reverse transcribed to cDNA, i.e., the proximity probe is bound directly to the target nucleic acid.

In embodiments, the biomolecules, otherwise referred to herein as targets, are proteins. In embodiments when the target are proteins, the method includes contacting the proteins with a plurality of proximity probes, wherein each proximity probe includes an oligonucleotide barcode (e.g., an oligonucleotide barcode associated with that particular target protein). In embodiments, the proximity probe includes an antibody, single-chain Fv fragment (scFv), antibody fragment-antigen binding (Fab), or an aptamer. In embodiments, the biomolecule is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. In embodiments, the biomolecule interacts (e.g., contacts, or binds) with one or more proximity probes on the cell surface. Cell surface biomolecules corresponding to analytes can include a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, or a posttranslational modification (e.g., phosphorylation, glycosylation, ubiquitination, nitrosylation, methylation, acetylation or lipidation).

In embodiments, the methods further includes imaging the cell (e.g., obtaining bright field images (i.e., transmitted light) or dark field images (i.e., scattered light). In embodiments, the method further includes identifying and/or quantifying additional targets of interest (e.g., proteins, nucleic acids, glycolipids, or cellular structures (e.g., nucleus, mitochondria, or organelles). In embodiments, the light transmittance of the sample is measured. For example, light transmittance may be measured with a visible near-infrared optical fiber spectrometer, wherein a circular spot of light (e.g., diameter, 5 mm) is irradiated on the central part a sample and the transmitted light is collected using an optical sensor. In embodiments, the method includes obtaining cell images for analysis of cell morphology. In embodiments, a plurality of cells are immobilized in a 96-well microplate having a mean or median well-to-well spacing of about 8 mm to about 12 mm (e.g., about 9 mm). In embodiments, a plurality of cells is immobilized in a 384-well microplate having a mean or median well-to-well spacing of about 3 mm to about 6 mm (e.g., about 4.5 mm). In embodiments, the device as described herein detects scattered light from the sample. In embodiments, the device as described herein detects diffracted light from the sample. In embodiments, the device as described herein detects reflected light from the sample. In embodiments, the device as described herein detects absorbed light from the sample. In embodiments, the device as described herein detects refracted light from the sample. In embodiments, the device as described herein detects transmitted light not absorbed by the sample. In embodiments, the sample does not include a label. In embodiments, the methods and system as described herein detect scattered light from the sample. In embodiments, the methods and system as described herein detect diffracted light from the sample. In embodiments, the methods and system as described herein detect reflected light from the sample. In embodiments, the methods and system as described herein detect absorbed light from the sample. In embodiments, the methods and system as described herein detect refracted light from the sample. In embodiments, the methods and system as described herein detect transmitted light not absorbed by the sample. In embodiments, the device is configured to determine the cell morphology (e.g., the cell boundary, granularity, or cell shape). For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013).

In embodiments, the cell is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a z-step interval of 1 to 5 μm. A typical light sheet fluorescence microscope includes an sCMOS camera, a 2×/0.5 objective lens, and zoom microscope body (magnification range of ×0.63 to ×6.3). For entire scanning of whole samples, the z-step interval is 5 or 10 m, and for image acquisition in the regions of interest, an interval in the range of 2 to 5 m may be used.

In embodiments, the method includes performing an additional image processing techniques (e.g., filtering, masking, smoothing, UnSharp Mask filter (USM), deconvolution, or maximum intensity projection (MIP)). In embodiments, the method includes computationally filtering the emissions using a linear or nonlinear filter that amplifies the high-frequency components of the emission. For example, USM method applies a Gaussian blur to a duplicate of the original image and then compares it to the original. If the difference is greater than a threshold setting, the images are subtracted. In embodiments, the method includes a maximum intensity projection (MIP). A maximum intensity projection is a visualization technique that takes three-dimensional data (e.g., emissions from varying depths obtained according to the methods described herein) and turns it into a single two-dimensional image. For example, the projection takes the brightest pixel (voxel) in each depth and displays that pixel intensity value in the final two-dimensional image. Various machine learning approaches may be used, for example, the methods described in Lugagne et al. Sci Rep 8, 11455 (2018) and Pattarone, G., et al. Sci Rep 11, 10304 (2021), each of which are incorporated herein by reference. In embodiments, the method includes focus stacking (e.g., z-stacking) which combines multiple images taken at different focus distances to give a resulting image with a greater depth of field (DOF) than any of the individual source images. The devices and methods described herein provide for the detection analytes and analyte levels (e.g., gene and/or protein expression) within different cells in a tissue of a mammal or within a single cell. For example, the methods can be used to detect analytes (e.g., genes and/or proteins) within different cells in histological slide samples, the data from which can be reassembled to generate a three-dimensional map of analytes of a tissue sample.

In embodiments, the method further includes sequencing the amplification product(s). Sequencing includes, for example, detecting a sequence of signals within the sample (e.g., within the cell or within the tissue). Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the readout is accomplished by epifluorescence imaging. A variety of sequencing chemistries are available, non-limiting examples of which are described herein.

In embodiments, sequencing includes extending a sequencing primer to incorporate a nucleotide containing a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product of a target nucleic acid). In embodiments, the sequencing includes sequencing-by-synthesis, sequencing-by-binding, sequencing by ligation, sequencing-by-hybridization, or pyrosequencing, and generates a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

In embodiments, the sequencing includes extending a sequencing primer by incorporating a labeled nucleotide or labeled nucleotide analogue, and detecting the label to generate a signal for each incorporated nucleotide or nucleotide analogue, wherein the sequencing primer is hybridized to the extension product.

In embodiments, the sequencing primer includes a reversible 3′ blocking moiety. In embodiments, the reversible blocking moiety includes a dideoxy nucleotide triphosphate. In embodiments, prior to hybridizing the sequencing primer to the extension product, the reversible blocking moiety is removed, thereby generating an extendible sequencing primer. In embodiments, the sequencing primer is immobilized to a matrix or a cellular component of the cell. In embodiments, the sequencing primer is immobilized to a solid support.

In embodiments, the one or more immobilized oligonucleotides (e.g., the one or more immobilized primers in a cell or on a solid support) include blocking groups at their 3′ ends that prevent polymerase extension. A blocking moiety prevents formation of a covalent bond between the 3′ hydroxyl moiety of the nucleotide and the 5′ phosphate of another nucleotide. A blocking moiety can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. Non-limiting examples of 3′ blocking groups include a 3′-ONH₂ blocking group, a 3′-O-allyl blocking group, or a 3′-O-azidomethyl blocking group. In embodiments, the 3′ blocking group is a C3, C9, C12, or C18 spacer phosphoramidite, a 3′phosphate, a C3, C6, C12 amino modifier, or a reversible blocking moiety (e.g., reversible blocking moieties are described in U.S. Pat. Nos. 7,541,444 and 7,057,026). In embodiments, the 3′ modification is a 3′-phosphate modification includes a 3′ phosphate moiety, which is removed by a PNK enzyme.

In embodiments, sequencing includes a plurality of sequencing cycles. In embodiments, sequencing includes 10 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, detecting includes two-dimensional (2D) or three-dimensional (3D) fluorescent microscopy. Suitable imaging technologies are known in the art, as exemplified by Larsson et al., Nat. Methods (2010) 7:395-397 and associated supplemental materials, the entire content of which is incorporated by reference herein in its entirety. In embodiments of the methods provided herein, the imaging is accomplished by confocal microscopy. Confocal fluorescence microscopy involves scanning a focused laser beam across the sample, and imaging the emission from the focal point through an appropriately-sized pinhole. This suppresses the unwanted fluorescence from sections at other depths in the sample. In embodiments, the imaging is accomplished by multi-photon microscopy (e.g., two-photon excited fluorescence or two-photon-pumped microscopy). Unlike conventional single-photon emission, multi-photon microscopy can utilize much longer excitation wavelength up to the red or near-infrared spectral region. This lower energy excitation requirement enables the implementation of semiconductor diode lasers as pump sources to significantly enhance the photostability of materials. Scanning a single focal point across the field of view is likely to be too slow for many sequencing applications. To speed up the image acquisition, an array of multiple focal points can be used. The emission from each of these focal points can be imaged onto a detector, and the time information from the scanning mirrors can be translated into image coordinates. Alternatively, the multiple focal points can be used just for the purpose of confining the fluorescence to a narrow axial section, and the emission can be imaged onto an imaging detector, such as a CCD, EMCCD, or s-CMOS detector. A scientific grade CMOS detector offers an optimal combination of sensitivity, readout speed, and low cost. One configuration used for confocal microscopy is spinning disk confocal microscopy. In 2-photon microscopy, the technique of using multiple focal points simultaneously to parallelize the readout has been called Multifocal Two-Photon Microscopy (MTPM). Several techniques for MTPM are available, with applications typically involving imaging in biological tissue. In embodiments of the methods provided herein, the imaging is accomplished by light sheet fluorescence microscopy (LSFM). In embodiments, detecting includes 3D structured illumination (3DSIM). In 3DSIM, patterned light is used for excitation, and fringes in the Moird pattern generated by interference of the illumination pattern and the sample, are used to reconstruct the source of light in three dimensions. In order to illuminate the entire field, multiple spatial patterns are used to excite the same physical area, which are then digitally processed to reconstruct the final image. See York, Andrew G., et al. “Instant super-resolution imaging in live cells and embryos via analog image processing.” Nature methods 10.11 (2013): 1122-1126, which is incorporated herein by reference. In embodiments, detecting includes selective planar illumination microscopy, light sheet microscopy, emission manipulation, pinhole confocal microscopy, aperture correlation confocal microscopy, volumetric reconstruction from slices, deconvolution microscopy, or aberration-corrected multifocus microscopy. In embodiments, detecting includes digital holographic microscopy (see for example Manoharan, V. N. Frontiers of Engineering: Reports on Leading-edge Engineering from the 2009 Symposium, 2010, 5-12, which is incorporated herein by reference). In embodiments, detecting includes confocal microscopy, light sheet microscopy, or multi-photon microscopy.

In embodiments, detecting includes contacting the target of interest (e.g., a nucleic acid, protein, or biomolecule) with a fluorescently labeled probe and detecting the probe following hybridization. In embodiments, detecting includes contacting the circularized product with a fluorescently labeled probe and detecting the probe following hybridization. In embodiments, detecting includes contacting the amplification product with a fluorescently labeled probe and detecting the probe following hybridization. In embodiments, detecting includes contacting the sample (e.g., the sample including the circularized product and/or amplification product) with an detection solution (e.g., a buffered solution including a detectable agent, such as a fluorescently labeled probe) for about 5 minutes to about 1 hour, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 1 hour, about 10 minutes to about 50 minutes, about 10 minutes to about 40 minutes, about 10 minutes to about 30 minutes, about 10 minutes to about 20 minutes, about 20 minutes to about 1 hour, about 20 minutes to about 50 minutes, about 20 minutes to about 40 minutes, about 20 minutes to about 30 minutes, about 30 minutes to about 1 hour, about 30 minutes to about 50 minutes, about 30 minutes to about 40 minutes, about 40 minutes to about 1 hour, about 40 minutes to about 50 minutes, or about 50 minutes to about 1 hour, at a temperature of about 4° C. to about 35° C., about 4° C. to about 30° C., about 4° C. to about 25° C., about 4° C. to about 20° C., about 4° C. to about 15° C., about 4° C. to about 10° C., about 10° C. to about 35° C., about 10° C. to about 30° C., about 10° C. to about 25° C., about 10° C. to about 20° C., about 10° C. to about 15° C., about 15° C. to about 35° C., about 15° C. to about 30° C., about 15° C. to about 25° C., about 15° C. to about 20° C., about 20° C. to about 35° C., about 20° C. to about 30° C., about 20° C. to about 25° C., about 25° C. to about 35° C., about 25° C. to about 30° C., or about 30° C. to about 35° C., and detecting the detectable agent of the detection solution. The phrase “labeled probes” refers to mixture of nucleic acids that are detectably labeled, e.g., fluorescently labeled, such that the presence of the probe, as well as any target sequence to which the probe is bound, can be detected by assessing the presence of the label. In some embodiments, the probes are about 30-300 bases in length, 40-300 bases in length, or 70-300 bases in length. In some embodiments, the probes are relatively uniform in length (e.g., an average length+/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety.

In embodiments, the method includes sequencing the first and/or the second strand of an amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which are incorporated herein by reference in their entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids and amplicons thereof that are present at features of an array are subjected to repeated cycles of oligonucleotide delivery and detection. SBL methods include those described in Shendure et al. Science 309:1728-1732 (2005); U.S. Pat. Nos. 5,599,675; and 5,750,341, each of which are incorporated herein by reference in their entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which are incorporated herein by reference in their entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by an SBS process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. No. 10,738,072. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, sequencing is performed according to a “sequencing-by-binding” method (see, e.g., U.S. Pat. Pubs. US2017/0022553 and US2019/0048404, each of which is incorporated herein by reference in its entirety), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

In embodiments, the circular polynucleotide includes an endogenous nucleic acid sequence, or a complement thereof. In embodiments, the circular polynucleotide includes a genomic sequence, or a complement thereof. In embodiments, the circular polynucleotide includes a synthetic sequence, or a complement thereof.

In embodiments, the method includes amplifying the circular polynucleotide of the cell in situ. In embodiments, amplifying the circular polynucleotide generates an amplification product. In embodiments, the amplification product includes three or more copies of the circular polynucleotide. In embodiments, the amplification product includes at least three or more copies of the circular polynucleotide. In embodiments, the amplification product includes at least five or more copies of the circular polynucleotide. In embodiments, the amplification product includes at 5 to 10 copies of the circular polynucleotide. In embodiments, the amplification product includes 10 to 20 copies of the circular polynucleotide. In embodiments, the amplification product includes 20 to 50 copies of the circular polynucleotide.

In embodiments, amplifying the circular polynucleotide includes incubating the circular polynucleotide with the strand-displacing polymerase (a) for about 1 minute to about 2 hours, and/or (b) at a temperature of about 20° C. to about 50° C. In embodiments, amplifying the circular polynucleotide includes incubating the circular polynucleotide with the strand-displacing polymerase for about 1 minute to about 2 hours. In embodiments, amplifying the circular polynucleotide includes incubating the circular polynucleotide with the strand-displacing polymerase for about 5, about 10, about 20, about 30, about 40, about 45, about 50, about 55, or about 60 minutes. In embodiments, amplifying the circular polynucleotide includes incubating the circular polynucleotide with the strand-displacing polymerase for about 5 minutes. In embodiments, amplifying the circular polynucleotide includes incubating the circular polynucleotide with the strand-displacing polymerase for about 10 minutes. In embodiments, amplifying the circular polynucleotide includes incubating the circular polynucleotide with the strand-displacing polymerase for about 20 minutes. In embodiments, amplifying the circular polynucleotide includes incubating the circular polynucleotide with the strand-displacing polymerase for about 30 minutes. In embodiments, amplifying the circular polynucleotide includes incubating the circular polynucleotide with the strand-displacing polymerase for about 45 minutes. In embodiments, amplifying the circular polynucleotide includes incubating the circular polynucleotide with the strand-displacing polymerase for about 60 minutes.

In embodiments, amplifying the circular polynucleotide includes incubating the circular polynucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular polynucleotide includes incubating the circular polynucleotide with the strand-displacing polymerase for about 1, about 2, about 3, about 4, about 5, about 6, about 7, about 8, about 9, about 10, about 11, or about 12 hours. In embodiments, amplifying the circular polynucleotide includes incubating the circular polynucleotide with the strand-displacing polymerase for more than 12 hours.

In embodiments, amplifying the circular polynucleotide includes incubating the circular polynucleotide with the strand-displacing polymerase at a temperature of about 20° C. to about 50° C. In embodiments, incubation with the strand-displacing polymerase is at a temperature of about 20° C., about 25° C., about 30° C., about 35° C., about 40° C., about 45° C., or about 50° C. In embodiments, incubation with the strand-displacing polymerase is at a temperature of about 35° C. to 42° C. In embodiments, incubation with the strand-displacing polymerase is at a temperature of about 35° C., about 36° C., about 37° C., about 38° C., about 39° C., about 40° C., about 41° C., or about 42° C. In embodiments, the strand-displacing polymerase is a phi29 polymerase, a SD polymerase, a Bst large fragment polymerase, phi29 mutant polymerase, a Thermus aquaticus polymerase, or a thermostable phi29 mutant polymerase.

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is Bst DNA Polymerase Large Fragment, Thermus aquaticus (Taq) polymerase, or a mutant thereof. In embodiments, the strand-displacing polymerase is a phi29 polymerase, a phi29 mutant polymerase or a thermostable phi29 mutant polymerase. A “phi polymerase” (or “Φ29 polymerase”) is a DNA polymerase from the (29 phage or from one of the related phages that, like Φ29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase). In embodiments, the polymerase is a phage or bacterial RNA polymerases (RNAPs). In embodiments, the polymerase is a T7 RNA polymerase. In embodiments, the polymerase is an RNA polymerase. Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaea RNA polymerase.

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g. a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)9)).

In embodiments, the circularizable oligonucleotide (e.g., the oligonucleotide primer) contains one or more functional moieties (e.g., bioconjugate reactive groups) that serve as attachment points to the cell (i.e., the internal cellular scaffold) or to the matrix in which the cell is embedded (e.g. a hydrogel). In embodiments, the bioconjugate reactive group is located at the 5′ and/or 3′ end of the oligonucleotide. In embodiments, the bioconjugate reactive group is located at an internal position of the oligonucleotide e.g., the oligonucleotide contains one or more modified nucleotides, such as aminoallyl deoxyuridine 5′-triphosphate (dUTP) nucleotide(s). In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, the oligonucleotide primer contains a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, prior to amplification, the modified nucleotide-containing primer is attached to the cell protein matrix by using a cross-linking reagent (e.g., an amine-reactive crosslinking agent with PEG spacers, such as (PEGylated bis(sulfosuccinimidyl)suberate) (BS(PEG)9)).

It will be appreciated that any of the amplification methodologies described herein or known in the art can be utilized with universal or target-specific primers to amplify the target polynucleotide ex situ (e.g., the one or more extended polynucleotides, or circularized probes, including two or more barcodes are removed from the sample, for example the cell or tissue, and amplified on a different solid support or in solution). Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest ex situ. In embodiments, amplification includes an isothermal amplification reaction. In embodiments, amplification includes bridge amplification. In general, bridge amplification uses repeated steps of annealing of primers to templates, primer extension, and separation of extended primers from templates. Because primers are attached within the core polymer, the extension products released upon separation from an initial template is also attached within the core. The 3′ end of an amplification product is then permitted to anneal to a nearby reverse primer that is also attached within the core, forming a “bridge” structure. The reverse primer is then extended to produce a further template molecule that can form another bridge. In embodiments, forward and reverse primers hybridize to primer binding sites that are specific to a particular target nucleic acid. In embodiments, forward and reverse primers hybridize to primer binding sites that have been added to, and are common among, target polynucleotides. Adding a primer binding site to target nucleic acids can be accomplished by any suitable method, examples of which include the use of random primers having common 5′ sequences and ligating adapter nucleotides that include the primer binding site.

In certain embodiments the term “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” or “cluster” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments of the methods provided herein, the amplicon clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μm or a number or a range between any two of these values. The mean or median separation may be measured center-to-center (i.e., the center of one amplicon cluster to the center of a second amplicon cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm.

In embodiments of the methods provided herein, the amplicon clusters have a mean or median diameter of about 100-2000 nm, or about 200-1000 nm. In embodiments, the mean or median diameter is about 100-3000 nanometers, about 500-2500 nanometers, about 1000-2000 nanometers, or a number or a range between any two of these values. In embodiments, the mean or median diameter is about or at most about 100, 200, 300, 400, 500, 600, 700, 800, 900, 1000, 1100, 1200, 1300, 1400, 1500, 1600, 1700, 1800, 1900, 2000 nanometers or a number or a range between any two of these values.

In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, amplifying includes a bridge polymerase chain reaction amplification. In embodiments, amplifying includes a thermal bridge polymerase chain reaction (t-bPCR) amplification. In embodiments, amplifying includes a chemical bridge polymerase chain reaction (c-bPCR) amplification. Chemical bridge polymerase chain reactions include fluidically cycling a denaturant (e.g., formamide) and one or more additives (e.g., ethylene glycol) and maintaining the temperature within a narrow temperature range (e.g., +/−5° C.) or isothermally. In embodiments, c-bPCR does not include isothermal amplification, rather it requires minor (e.g., +/−5° C.) thermal oscillations. In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. In embodiments, amplifying includes generating a double-stranded amplification product.

In embodiments, amplifying a template polynucleotide generates amplification products. In embodiments, amplifying includes a plurality of cycles of strand denaturation, primer hybridization, and primer extension. In embodiments, amplifying includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In embodiments, the total volume of the cell is about 1 to 25 μm³. In embodiments, the volume of the cell is about 5 to 10 μm³. In embodiments, the volume of the cell is about 3 to 7 μm³.

In embodiments, the optically resolved volume has an axial resolution (i.e., depth, or z) that is greater than the lateral resolution (i.e., xy plane). In embodiments, the optically resolved volume has an axial resolution that is greater than twice the lateral resolution. In embodiments, the dimensions (i.e., the x, y, and z dimensions) of the optically resolved volume are about 0.5 μm×0.5 μm×0.5 μm; 1 μm×1 μm×1 μm; 2 μm×2 μm×2 μm; 0.5 μm×0.5 μm×1 μm; 0.5 μm×0.5 μm×2 μm; 2 μm×2 μm×1 μm; or 1 μm×1 μm×2 μm. In embodiments, the dimensions (i.e., the x, y, and z dimensions) of the optically resolved volume are about 1 μm×1 μm×2 μm; 1 μm×1 μm×3 μm; 1 μm×1 μm×4 μm; or about 1 μm×1 μm×5 μm. In embodiments, the dimensions (i.e., the x, y, and z dimensions) of the optically resolved volume are about 1 μm×1 μm×5 μm. In embodiments, the dimensions (i.e., the x, y, and z dimensions) of the optically resolved volume are about 1 μm×1 μm×6 μm. In embodiments, the dimensions (i.e., the x, y, and z dimensions) of the optically resolved volume are about 1 μm×1 μm×7 μm. In embodiments, the optically resolved volume is a cubic micron. In embodiments, the optically resolved volume has a lateral resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has an axial resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has an axial resolution from about 1 to 2 m, from 2 to 3 m, from 3 to 4 m, from 4 to 5 m, from 5 to 6 m, or from 6 to 10 μm.

In embodiments, the method further includes an additional imaging modality, immunofluorescence (IF), or immunohistochemistry modality (e.g., immunostaining). In embodiments, the method includes ER staining (e.g., contacting the cell with a cell-permeable dye which localizes to the endoplasmic reticula), Golgi staining (e.g., contacting the cell with a cell-permeable dye which localizes to the Golgi), F-actin staining (e.g., contacting the cell with a phalloidin-conjugated dye that binds to actin filaments), lysosomal staining (e.g., contacting the cell with a cell-permeable dye that accumulates in the lysosome via the lysosome pH gradient), mitochondrial staining (e.g., contacting the cell with a cell-permeable dye which localizes to the mitochondria), nucleolar staining, or plasma membrane staining. For example, the method includes live cell imaging (e.g., obtaining images of the cell) prior to or during fixing, immobilizing, and permeabilizing the cell. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology (e.g., the cell boundary or cell shape) using known methods in the art. For example, determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013).

In aspects and embodiments described herein, the methods are useful in the field of predictive medicine in which diagnostic assays, prognostic assays, pharmacogenomics, and monitoring clinical trials are used for prognostic (i.e., predictive) purposes to thereby treat an individual prophylactically. Accordingly, in embodiments the methods of diagnosing and/or prognosing one or more diseases and/or disorders using one or more of expression profiling methods described herein are provided.

In embodiments, the method includes fixing and/or staining the sample. In embodiments of any of the methods described herein, the non-permeabilized biological sample is fixed and/or stained prior. In embodiments, the step of fixing the sample includes the use of a fixative (e.g., contacting and/or incubating with the sample) such as ethanol, methanol, acetone, formaldehyde, paraformaldehyde-Triton, glutaraldehyde, and combinations thereof. In embodiments, the staining the sample includes contacting and/or incubating with the sample acridine orange, Bismarck brown, carmine, coomassie blue, cresyl violet, DAPI, eosin, ethidium bromide, acid fuchsin, hematoxylin, Hoechst stains, iodine, methyl green, methylene blue, neutral red, Nile blue, Nile red, osmium tetroxide, propidium iodide, rhodamine, safranin, and combinations thereof. In embodiments, staining includes contacting the sample with eosin and hematoxylin. In embodiments, staining includes contacting the sample with a detectable label selected from the group consisting of a radioisotope, a fluorophore, a chemiluminescent compound, a bioluminescent compound, or a combination thereof.

The biological targets or molecules to be detected can be any biological molecules including but not limited to proteins, nucleic acids, lipids, carbohydrates, ions, or multicomponent complexes containing any of the above. Examples of subcellular targets include organelles, e.g., mitochondria, Golgi apparatus, endoplasmic reticulum, chloroplasts, endocytic vesicles, exocytic vesicles, vacuoles, lysosomes, etc. Exemplary nucleic acid targets can include genomic DNA of various conformations (e.g., A-DNA, B-DNA, Z-DNA), mitochondria DNA (mtDNA), mRNA, tRNA, rRNA, hRNA, miRNA, and piRNA.

In embodiments, the collection of information (e.g., sequencing information and/or cell morphology) is referred to as a signature. The term “signature” may encompass any gene or genes, protein or proteins, or epigenetic element(s) whose expression profile or whose occurrence is associated with a specific cell type, subtype, or cell state of a specific cell type or subtype within a population of cells. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity of signatures may be compared between different cells in order to characterize or identify for instance specific cell (sub)populations.

In embodiments, the methods described herein may further include constructing a 3-dimensional pattern of abundance, expression, and/or activity of each target from spatial patterns of abundance, expression, and/or activity of each target of multiple samples. In embodiments, the multiple samples can be consecutive tissue sections of a 3-dimensional tissue sample.

In embodiments, the method further includes removing the embedding material from the sample. For example, if the embedding material is paraffin wax, the embedding material is removed by contacting the sample-carrier construct with a hydrocarbon solvent, such as xylene or hexane, followed by two or more washes with decreasing concentrations of an alcohol, such as ethanol.

The methods can be used to characterize a cancer or metastasis thereof, including without limitation, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangioendothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma. Lymphoma and leukemia include without limitation chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma. Germ cell tumors include without limitation germinoma, dysgerminoma, seminoma, nongerminomatous germ cell tumor, embryonal carcinoma, endodermal sinus tumor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In embodiments, the method includes imaging the immobilized tissue section. In embodiments, the method further includes an imaging modality, immunofluorescence (IF), or immunohistochemistry modality (e.g., immunostaining). In embodiments, the method includes ER staining (e.g., contacting the tissue section with a cell-permeable dye which localizes to the endoplasmic reticula), Golgi staining (e.g., contacting the tissue section with a cell-permeable dye which localizes to the Golgi), F-actin staining (e.g., contacting the tissue section with a phalloidin-conjugated dye that binds to actin filaments), lysosomal staining (e.g., contacting the tissue section with a cell-permeable dye that accumulates in the lysosome via the lysosome pH gradient), mitochondrial staining (e.g., contacting the tissue section with a cell-permeable dye which localizes to the mitochondria), nucleolar staining, or plasma membrane staining. For example, the method includes live cell imaging (e.g., obtaining images of the tissue section) prior to or during fixing, immobilizing, and permeabilizing the tissue section. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology of the tissue section (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen.

In embodiments, additional methods may be performed to further characterize the sample. For example, in addition to sequencing, the method includes protein analysis, lipid analysis, metabolite analysis (e.g., glucose analysis), or measuring the transcriptomic profile, gene expression activity, genomic profile, protein expression activity, proteomic profile, protein interaction activity, cellular receptor expression activity, lipid profile, lipid activity, carbohydrate profile, microvesicle activity, glucose activity, and combinations thereof.

It will be appreciated that a barcode sequence and a complement of the barcode sequence, as described in the methods and compositions herein, are equivalent, in that if one sequence is known then the other sequence may be deduced and/or inferred.

### EXAMPLES

### Example 1. Detecting De Novo Proximal Protein Complexes In Situ

Early biological experiments revealed proteins as the main agents of biological function. As such, proteins ultimately determine the phenotype of all organisms. Proteins do not function in isolation; instead, it is their interactions with one another and also with other molecules (e.g., DNA, RNA, hormones, carbohydrates) that mediate metabolic and signaling pathways, cellular processes, and organismal systems. The concept of “protein interaction” is generally used to describe the physical contact between proteins and their interacting partners and any subsequent downstream effects. Proteins typically interact in pairs to form dimers (e.g., reverse transcriptase), multi-protein complexes (e.g., the proteasome for molecular degradation), or long chains (e.g., actin filaments in muscle fibers). The subunits creating the various complexes can be identical or heterogeneous (e.g., homodimers vs. heterodimers) and the duration of the interaction can be transient (e.g., proteins involved in signal transduction) or permanent (e.g., some ribosomal proteins). Historically, the main source of knowledge about protein interactions has come from biophysical methods, particularly from those based on deducing information based on structural information (e.g., X-ray crystallography, NMR spectroscopy, fluorescence, and/or atomic force microscopy) (see, Gonzalez M W and Kann M G. PLoS Comput. Biol. 2012; 8(12):e1002819, which is incorporated herein by reference in its entirety). Biophysical methods can identify interacting partners, and also provide detailed information about the biochemical features of the interactions, such as binding mechanism and allosteric changes involved. Yet, since they are time- and resource-consuming, biophysical characterizations only permit the study of a few complexes at a time, typically without any spatial information about the cellular or tissue-specific localization of a protein complex.

Protein biomarker discovery enables identification of signatures with pathophysiological importance, bridging the gap between genomes and phenotypes. This type of data may have a profound impact on improving future healthcare, particularly with respect to precision medicine, but progress has been hampered by the lack of technologies that can provide reliable specificity, high throughput, sufficient precision, and high sensitivity. Expanding the knowledge of cellular protein interaction networks is vital to improve our understanding of several types of diseases, including cancer. Improved methods to study these interaction networks, especially in clinical settings, is therefore of great importance both for increasing the knowledge of the underlying disease mechanics, but also for finding new biomarkers for improved disease diagnostics and treatment response prediction. Another context where multiplexed detection of protein-protein interactions provides decisive importance is in the field of network pharmacology, where drugs are designed to act on several drug targets simultaneously. The rationale being that as cellular interaction networks are quite robust because of their underlying structure, to perturb these networks and to avoid escape mutations in malignancy, it may prove crucial to target several proteins simultaneously.

There is a need for new methods that can provide information on more than isolated protein interaction events, such as the simultaneous detection of several interactions. Such methods can uncover protein interaction networks, aid in understanding protein complex architectures in tissue-specific contexts, and provide better diagnostics and treatment options. Existing in situ proteomic assays provide information on protein expression and cellular localization, while sequencing information is obtained ex situ. Beyond quantifying protein expression data, obtaining precise information about the identity and localization of protein complexes will support the identification of malignancies, monitor aberrant protein activity, and support the development of targeted treatments at the molecular level. Disclosed herein are solutions to these and other problems in the art.

Mammalian cells are organized into different compartments that separate and facilitate physiological processes by providing specialized local environments and allowing different, otherwise incompatible biological processes to be carried out simultaneously. Proteins are targeted to these subcellular locations where they fulfill specialized, compartment-specific functions. Spatial proteomics aim to localize and quantify proteins within subcellular structures to provide three important biological insights. Firstly, spatial proteomics enables placing a protein in a specific location within the cell provides a hypothesis about what function the protein might have. For example, proteins localized to the mitochondria could have roles in energy production or apoptosis. Secondly, it can indicate a specific state of the cell or provide potential hypotheses about a new function of a protein if the protein is found in different subcellular locations simultaneously or upon perturbation. Thirdly, determining the localization of proteins is important to understand the functions of organelles and compartments. Most importantly, spatial proteomics of the non-perturbed state also provides a baseline for detecting aberrant localization of proteins, which is an important cause for a number of different human diseases (see, Pankow S et al. Curr. Opin. Chem. Biol. 2019; 48:19-25).

Studies of the human proteome have begun to reveal a complex architecture, including single-cell variations, dynamic protein translocations, changing interaction networks, and proteins locating to multiple compartments. A typical human cell expresses more than 10,000 different proteins, spanning an abundance range of seven orders of magnitude. Current large-scale studies of the human spatial proteome suggest that it has a highly complex architecture that includes single-cell variation (in both protein level and localization), dynamic protein translocation, changing interaction networks and the localization of approximately half of all proteins to multiple compartments. The incorporation of global quantification data enables cellular model building and systems analyses that go beyond qualitative descriptions. Furthermore, several studies have successfully harnessed the power of global spatial proteomics to investigate diseases, including acute viral infection and liver disease, or to pinpoint the cellular defects that underlie monogenic disorders (see, e.g., Lundberg E and Borner G H H. Nat. Rev. Mol. Cell. Biol. 2019; 20(5):285-302).

Sensitive detection of protein interactions and post-translational modifications of native proteins is a challenge for research and diagnostic purposes. A method for this, which could be used in point-of-care devices and high-throughput screening, should be reliable, cost effective and robust. Existing in situ proteomic assays provide information on protein expression and cellular localization, typically with the use of fluorophore-labeled probes or enzyme-labeled oligonucleotides, while any associated sequencing information (e.g., sequencing of an antibody-conjugated oligonucleotide barcode) is obtained ex situ, therein losing any spatial proteomic information. Two approaches used for detecting protein interactions in situ include the proximity ligation assay (PLA) and the proximity extension assay (PEA). Proximity probes include protein binding domain, such as antibodies or aptamers. Examples of aptamer affinity probes may be found in, for example, Fredriksson S et al. Nat. Biotechnol. 2002; 20(5):473-477.

Proximity ligation assay (PLA) combines multiple recognition events with potent signal amplification. The method is based on pairs of proximity probes (that is, antibodies conjugated to strands of DNA) to detect the proteins of interest (see, e.g., Alam M. Curr. Protoc. Immunol. 2018; 123(1): e58, which is incorporated herein by reference in its entirety). PLA assays have been commercialized, for example as Duolink® PLA technology from Sigma. Only on proximal binding of these probes can an amplifiable DNA strand be generated by ligation, which then is amplified by PCR. For localized detection, rolling circle amplification (RCA), an isothermal DNA amplification technique, may be used. RCA amplifies a circular template and generates long DNA strands that collapse into bundles of DNA. These bundles can be visualized by hybridizing fluorophore-labelled oligonucleotides to them and quantifying the number and intensity of dots by fluorescence microscopy, or by enzyme-labeled detection oligonucleotides, making it possible to detect single molecules in situ (see, e.g., Klaesson A et al. Sci. Rep. 2018; 8(1):5400, which is incorporated herein by reference in its entirety).

Proximity extension assay (PEA) typically utilizes two matched antibodies (e.g., two antibodies targeting the same protein) labelled with unique DNA oligonucleotides that simultaneously bind to a target protein in solution (for example, as commercialized by the Olink® PEA platform; for additional information on PEA see, e.g., International Patent Pub. Nos. WO 01/61037, WO 03/044231, WO 2004/094456, WO 2005/123963, WO 2006/137932, WO 2013/113699, and WO 2021/191442, each of which are incorporated herein by reference in their entirety). This brings the two antibodies into proximity, allowing their DNA oligonucleotides to hybridize, serving as template for a DNA polymerase-dependent extension step. This creates a double-stranded DNA “barcode” which is unique for the specific antigen and quantitatively proportional to the initial concentration of target protein. The hybridization and extension are immediately followed by PCR amplification. The resulting DNA amplicon can then be quantified either by qPCR, or by NGS-based approaches, depending on the specific protocol used. The exponential amplification properties of PCR are utilized in PEA to achieve a strong readout signal, providing assay sensitivity on par or better than traditional enzyme-linked immunosorbent assays (ELISAs). Importantly, this also means that extremely small sample volumes are needed to measure large numbers of proteins simultaneously, which is greatly beneficial when precious samples are in limited supply, such as in studies using human samples from clinical cohorts or biobank material (see, e.g., Lundberg M et al. Nucleic Acids Res. 2011; 39(15):e102 and Assarsson E et al. PLoS ONE. 2014; 9(4):e95129, each of which is incorporated herein by reference). A significant limitation of existing PLA and PEA methods for protein interaction detection, though, is that a priori knowledge of the protein targets of interest is required to select, for example, the antibodies of interest. These methods may therefore require additional validation of protein interactions, or complementary data from other experiments, such as mass spectrometry analysis of protein complexes, prior to performing.

There is a need for new methods that can provide information on more than isolated protein interaction events, such as the simultaneous detection of several interactions. Such methods can uncover protein interaction networks, aid in understanding protein complex architectures in tissue-specific contexts and provide better diagnostics and treatment options. Beyond quantifying protein expression data, obtaining precise information about the identity and localization of protein complexes will support the identification of malignancies, monitor aberrant protein activity, and support the development of targeted treatments at the molecular level. The compositions and methods described herein provide sequence-level resolution of protein interactions while retaining spatial information. Additionally, these methods allow for de novo identification of protein interaction networks in an in situ context, providing significantly more information than existing proteomic methods which either require known targets to be used or which require sequencing of target antibody barcodes to be performed ex situ, losing any spatial information.

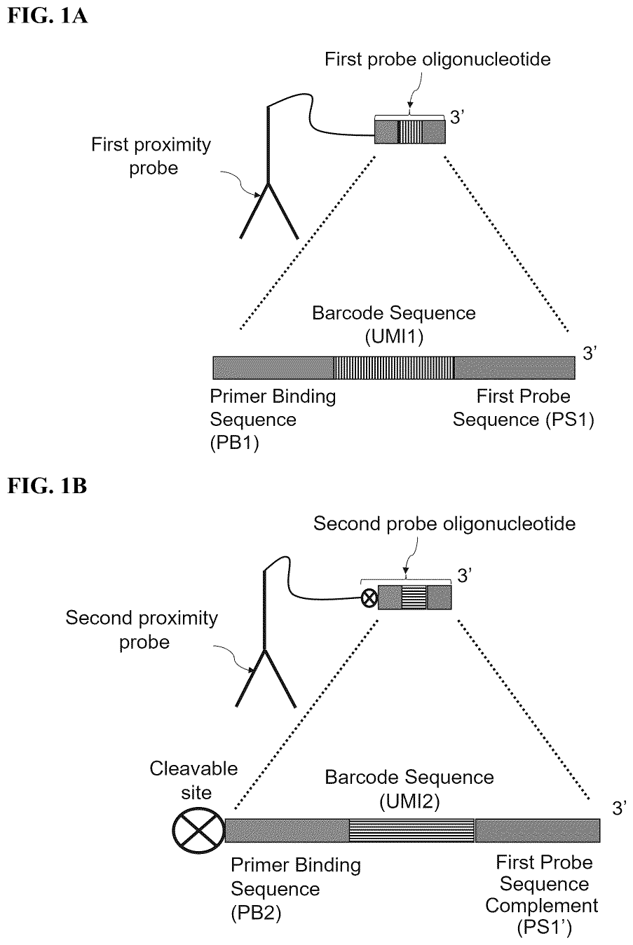

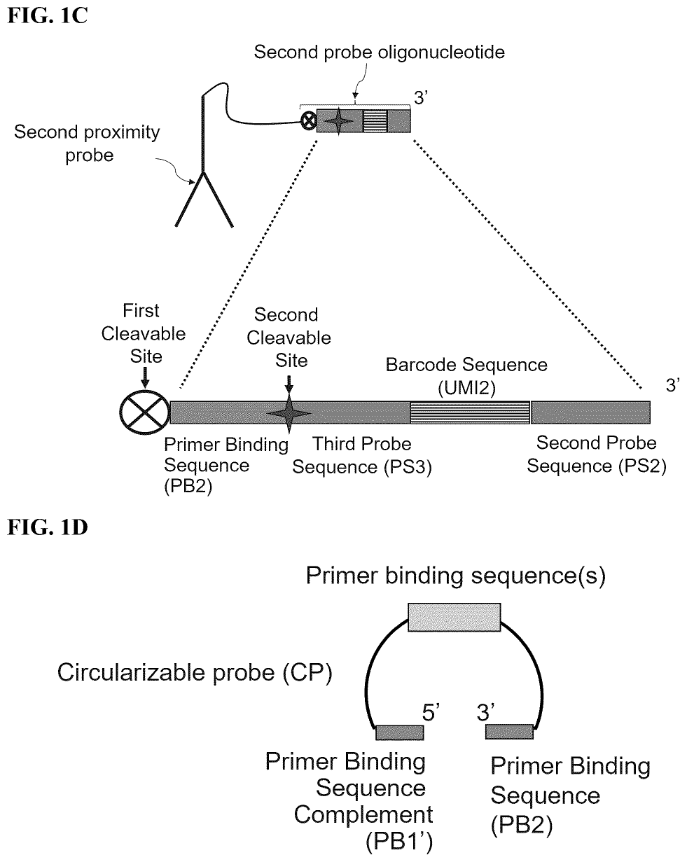

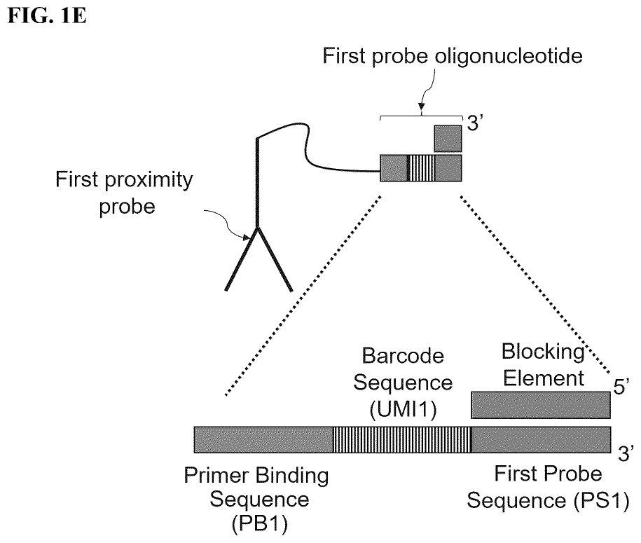

The approach described herein utilizes proximity probes, consisting of an analyte-binding domain, for example an aptamer or an antibody (e.g., a polyclonal or monoclonal antibody), that is conjugated to a multi-domain probe oligonucleotide. The proximity probes are designated as either “primary target probes” or “secondary target probes”, denoting the composition of the probe oligonucleotide conjugated to the probe. FIGS. 1A-1E illustrate embodiments of proximity probes (e.g., oligonucleotide-conjugated antibodies). FIG. 1A shows an embodiment of an oligonucleotide-conjugated proximity probe, referred to herein as a first proximity probe (or also referred to as a primary proximity probe). The first proximity probe includes a specific binding molecule (e.g., an antibody, affimer, aptamer, etc.) linked to a first probe oligonucleotide (also referred to herein as a first oligonucleotide or a primary probe oligonucleotide). The first probe oligonucleotide includes, from 5′ to 3′, a first primer binding sequence (PB1; also referred to herein as a first padlock probe (PLP) binding sequence), a first barcode sequence (UMI1; also referred to herein as a first unique molecular identifier), and a first probe sequence (PS1; also referred to herein as a first oligo interaction sequence). FIG. 1B shows an embodiment of a second proximity probe (or also referred to as a secondary proximity probe). The secondary proximity probe includes a specific binding molecule (e.g., an antibody, affimer, aptamer, etc.) linked to a second probe oligonucleotide (also referred to herein as a second oligonucleotide or a secondary probe oligonucleotide). The second probe oligonucleotide includes, from 5′ to 3′, a cleavable site, a second primer binding sequence (PB2; also referred to herein as a second padlock probe (PLP) binding sequence), a second barcode sequence (UMI2; also referred to herein as a second unique molecular identifier), and a complement to the first probe sequence (PS1′). FIG. 1C illustrates an alternate embodiment of a second proximity probe that includes two orthogonal cleavable sites. The second probe oligonucleotide includes, from 5′ to 3′, a first cleavable site, a second primer binding sequence (PB2), a second internal cleavable site, a third probe sequence (PS3; also referred to herein as a third oligo interaction sequence), a second barcode sequence (UMI2), and a second probe sequence (PS2; also referred to herein as a second oligo interaction sequence). The second cleavable site (also referred to herein as a second internal cleavable site) may be cleaved by an orthogonal mechanism to the first cleavable site (e.g., the first cleavable site is cleaved by a RNAse and the second internal cleavable site is cleaved by a restriction endonuclease). FIG. 1D illustrates a circularizable probe (CP; also referred to herein as a padlock probe or gap-fill padlock probe). The circularizable probe includes, from 5′ to 3′, a first primer binding sequence complement (PB1′), optionally, one or more primer binding sequences (e.g., one or more sequencing primer binding sequences and/or one or more amplification primer binding sequences), and a second primer binding sequence (PB2), wherein, for example, the PB1′ sequence of the circularizable probe is complementary to the PB1 sequence of the first probe oligonucleotide, and the PB2 sequence of the circularizable probe is complementary to the PB2′ sequence of the second probe oligonucleotide, as described herein. FIG. 1E illustrates an embodiment of the first proximity probe described in FIG. 1A, wherein the probe sequence (PS1) is hybridized to a blocking element, thereby preventing non-specific hybridization of the probe sequence and complement of the probe sequence on the first and second probe oligonucleotides. As illustrated in FIGS. 2A-2D, the proximity probes described herein may be used to detect two or more proteins present in a complex in situ. Additionally, as shown in FIG. 2B, the same approach may be used to detect single proteins through the use of two proximity probes targeting the same protein. In contrast to existing methods for profiling protein expression, the methods described herein allow for parallel sequencing-based detection in situ and spatial profiling, including de novo biomolecular interactions.

### Example 2. Spatial Detection of Binary Protein Complexes

Proximity probes of the art are generally used in pairs, and individually consist of an analyte-binding domain with specificity to the target analyte, and a nucleic acid domain coupled thereto. The analyte-binding domain can be, for example, a nucleic acid “aptamer” (Fredriksson et al (2002) Nat Biotech 20:473-477) or can be proteinaceous, such as a monoclonal or polyclonal antibody (Gullberg et al (2004) Proc Natl Acad Sci USA 101:8420-8424). The respective analyte-binding domains of each proximity probe pair may have specificity for different binding sites on the analyte, which analyte may consist of a single molecule or a complex of interacting molecules, or may have identical specificities, for example in the event that the target analyte exists as a multimer. When a proximity probe pair come into close proximity with each other, which will primarily occur when both are bound to their respective sites on the same analyte molecule, the nucleic acid domains are able to be joined to form a new nucleic acid sequence by means of a ligation reaction templated by a splint oligonucleotide subsequently added to the reaction, where the splint oligonucleotide contains regions of complementarity for the ends of the respective nucleic acid domains of the proximity probe pair. The new nucleic acid sequence thereby generated serves to report the presence or amount of analyte in a sample, and can be qualitatively or quantitatively detected, for example by real-time, quantitative PCR (q-PCR).

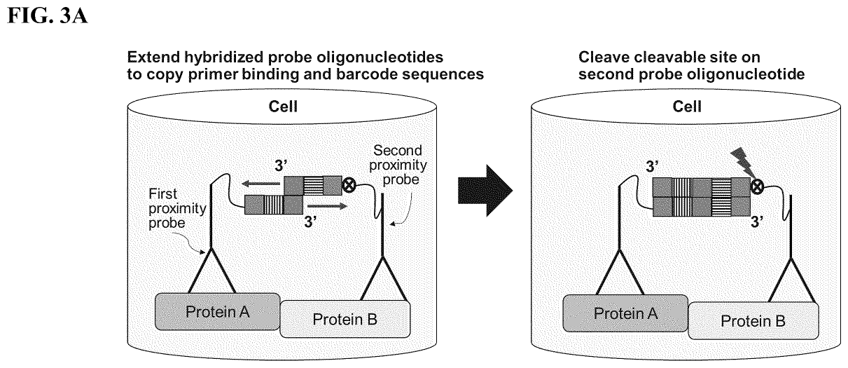

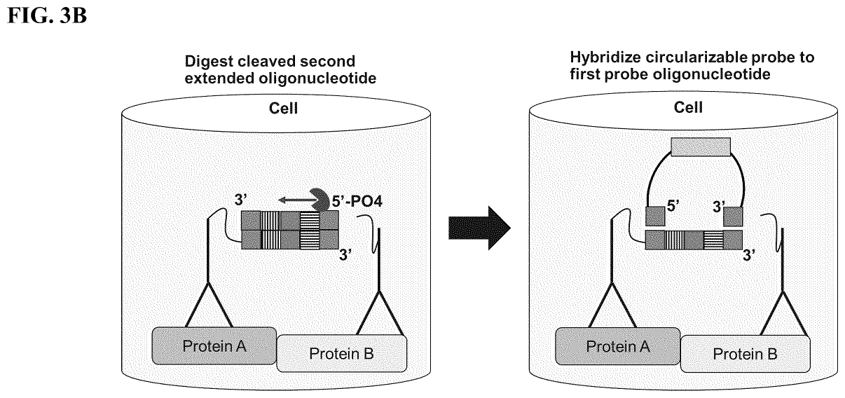

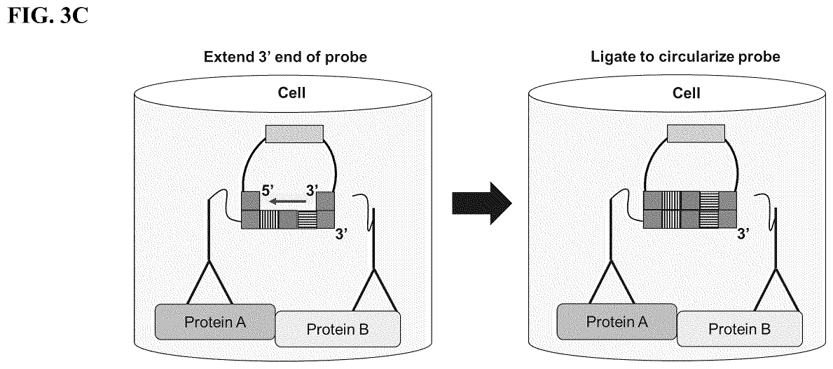

In situ sequencing involves tissue and/or cellular extraction, combined with the fixation and permeabilization of cells, followed by amplification of the target nucleic acid fragments for sequencing. Briefly, cells and their surrounding milieu are attached to a substrate surface, fixed, and permeabilized using known methods. FIGS. 3A-3D illustrate an embodiment of a method described herein for spatial detection of protein interactions using the proximity probes (e.g., oligonucleotide-conjugated antibodies) described herein. FIG. 3A illustrates a protein complex in a cell, wherein the complex includes Protein A bound to Protein B. A first proximity probe is bound to Protein A and is proximal to a second proximity probe bound to Protein B, such that the first and second probe oligonucleotides hybridize, as described in FIG. 2A. Using a polymerase, the 3′ end of each hybridized probe oligonucleotide is extended, generating a first extended oligonucleotide conjugated to the first proximity probe including, from 5′ to 3′, a first primer binding sequence (PB1), a first barcode sequence (UMI1), a first probe sequence (PS1), a complement of the second barcode sequence (UMI2′), and a complement of the second primer binding sequence (PB2′), and a second extended oligonucleotide conjugated to the secondary proximity antibody including, from 5′ to 3′, a second primer binding sequence (PB2), a second barcode sequence (UMI2), a complement of the first probe sequence (PS1′), a complement of the first barcode sequence (UMI1′), and a complement to the first primer binding sequence (PB1′). The cleavable site on the second probe oligonucleotide is then cleaved (e.g., RNAse cleavage of a ribonucleotide at or near the 5′ end of the second probe oligonucleotide), releasing the strand from the proximity probe (e.g., the antibody). In embodiments, the cleavable site is located in the linker between the specific binding molecule (e.g., antibody) and the probe oligonucleotide, rather than at the 5′ end of the secondary probe oligonucleotide.

FIG. 3B illustrates the steps of removing the cleaved strand (e.g., by lambda exonuclease 5′ to 3′ digestion), and subsequently hybridizing a circularizable probe onto the target nucleic acid sequence, wherein the PB1′ region at the 5′ end of the probe anneals to the PB1 sequence of the oligonucleotide, and wherein the PB2 region at the 3′ end anneals to the PB2′ sequence of the oligonucleotide. FIG. 3C illustrates the steps of extending the 3′ end of the circularizable probe (e.g., using a non-strand displace polymerase) to generate a complementary sequence, including from 3′ to 5′, the second barcode sequence (UMI2), the complement of the first probe sequence (PS1′), and the complement of the first barcode sequence (UMI1′). Following extension, the 3′ end of the complementary sequence is ligated to the 5′ end of the circularizable probe using, for example, a ligase, thereby generating a circularized probe. FIG. 3D illustrates the steps of amplifying the circularized probe (e.g., by rolling circle amplification using a processive strand-displacing polymerase), thereby generating a concatemer of amplification products. The amplification products are then detected, for example, by hybridizing a sequencing primer to a plurality of sequencing primer binding sequences on the amplification product, incorporated a labeled nucleotide (shown as a star) with a polymerase (shown as a cloud-like object), and detecting the label to identify the incorporated base. The amplification products may also be detected using fluorescently labeled probes.

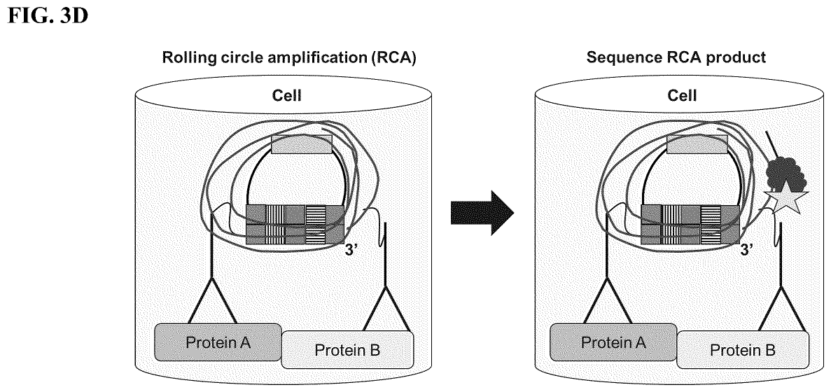

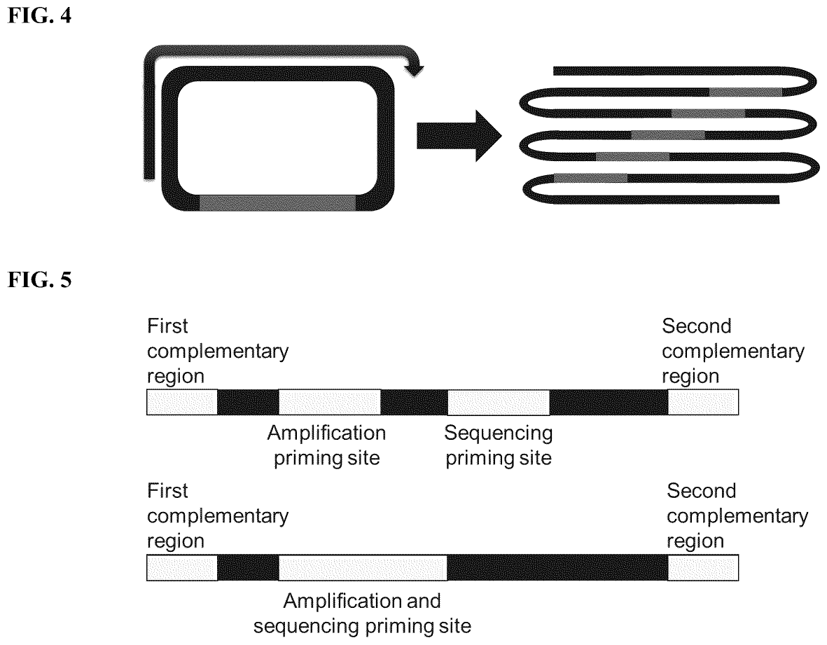

FIG. 4 illustrates a circularized probe (e.g., of FIG. 3C), primed with an amplification primer and extended with a strand-displacing polymerase to generate a concatemer containing multiple copies of the target nucleic acid sequence. As illustrated in FIG. 5, the padlock probe (PLP) is a single-stranded oligonucleotide containing a first complementary region and a second complementary region (i.e., nucleic acid sequences complementary to nucleic acid sequences flanking the target nucleic acid sequence). In embodiments, the padlock probe further includes an amplification priming site (i.e., a nucleic acid sequence complementary to an amplification primer) and a distinct sequencing priming site (i.e., a nucleic acid sequence complementary to a sequencing primer). Alternatively, in embodiments, the padlock probe further includes an amplification priming site and a sequencing priming site that are the same, are partially overlapping, or in which one is internal to the other. The amplification products are then detected, for example, by hybridizing a sequencing primer to a plurality of sequencing primer binding sequences on the amplification product, incorporated a labeled nucleotide (shown as a star) with a polymerase (shown as a cloud-like object), and detecting the label to identify the incorporated base. Alternative modes of detection are contemplated herein, for example FISH, SBB, and the like. In embodiments, the primer binding sequence is complementary to a fluorescent in situ hybridization (FISH) probe. FISH probes may be custom designed using known techniques in the art, see for example Gelali, E., et al. Nat Commun 10, 1636 (2019). Additional methods based on single molecule fluorescence in situ hybridization may also be used for detection. These include MERFISH (Multiplexed Error-Robust Fluorescence In Situ Hybridization), STARmap (Spatially-resolved Transcript Amplicon Readout mapping), FISSEQ, BaristaSeq, seq-FISH (Sequential Fluorescence In Situ Hybridization) and others (see for example Chen, K. H., et al. (2015). Science, 348(6233), aaa6090; Wang, G., Moffitt, J. R. & Zhuang, X. Sci Rep. 2018; 8, 4847; Wang X. et al; Science, 2018; 27, Vol 361, Issue 6400, eaat5691; Cai, M. Dissertation, (2019) UC San Diego. ProQuest ID: Cai_ucsd_0033D_18822; and Sansone, A. Nat Methods 16, 458; 2019).

The methods described herein provide a novel way to obtain a comprehensive in situ view of protein interactions without the need to perform ex situ sequencing or use laborious and expensive techniques such as mass spectrometry. The barcoded proximity probes can be scaled up or down to target numerous protein complexes in a sample. The methods provide unique insight into the spatial localization of protein complexes, for example, how protein complex components may vary depending on the tissue or cell under investigation, or under disease conditions.

### Example 3. Spatial Detection of Cellular Protein Interactomes

Spatial proteomics aims to localize and quantify proteins within subcellular structures to provide three important biological insights. Firstly, spatial proteomics enable placing a protein in a specific location within the cell provides a hypothesis about what function the protein might have. For example, proteins localized to the mitochondria could have roles in energy production or apoptosis. Secondly, it can indicate a specific state of the cell or provide potential hypotheses about a new function of a protein if the protein is found in different subcellular locations simultaneously or upon perturbation. Thirdly, determining the localization of proteins is important to understand the functions of organelles and compartments. Most importantly, spatial proteomics of the non-perturbed state also provides a baseline for detecting aberrant localization of proteins, which is an important cause for a number of different human diseases. Because spatial proteomics typically requires the enrichment of proteins prior to identification, results are fundamentally limited with regards to several basic aspects in subcellular biology of proteins (see, Pankow S et al. Curr. Opin. Chem. Biol. 2019; 48:19-25). Differences in protein abundance and localization can be dynamic and observed across macro-, meso-, and microscopic scales of tissues and cells. Existing methods for detecting cellular proteomes involve complex and expensive workflows, such as performing tandem-affinity purification of affinity tag-labeled proteins followed by mass spectrometry (see, e.g., Adelmant G et al. Curr. Protoc. Protein Sci. 2019; 96(1):e84, which is incorporated herein by reference in its entirety). New methods are needed to assess protein interactomes in situ, retaining spatial information while providing high-resolution identification of novel protein complexes.

In situ sequencing involves tissue and/or cellular extraction, combined with the fixation and permeabilization of cells, followed by amplification of the target nucleic acid fragments for sequencing. Briefly, cells and their surrounding milieu are attached to a substrate surface, fixed, and permeabilized using known methods. FIGS. 6A-6F illustrate an embodiment of the methods described herein for detecting a protein complex in situ using the proximity probes (e.g., oligonucleotide-conjugated antibodies) described herein. FIG. 6A illustrates a protein complex in a cell including Protein A, Protein B, and Protein C. A first proximity probe (as described in FIG. 1A) is bound to Protein A, and a second proximity probe and third proximity probe (each as described in FIG. 1C, each including both a first cleavable site and a second internal cleavable site), wherein the second proximity probe is bound to Protein B and the third proximity probe is bound to Protein C. Under conditions suitable for hybridization of the probe oligonucleotides (e.g., a buffered solution of suitable ionic strength for nucleic acid hybridization), two different probe oligonucleotide duplexes are possible between the first proximity probe bound to Protein A and either the second proximity probe bound to Protein B or the third proximity probe bound to Protein C.

FIG. 6B illustrates extension of the annealed Protein A and Protein C probe oligonucleotides, wherein the first probe sequence (1) of the first probe oligonucleotide is duplexed to the second probe sequence (2) of the second probe oligonucleotide. Using a polymerase, the 3′ end of each hybridized probe oligonucleotide is extended, generating: a first extended oligonucleotide conjugated to the first proximity probe including, from 5′ to 3′, a first primer binding sequence (PB1), a first barcode sequence (UMI1), the first probe sequence (1), a complement to the second barcode sequence (UMI2′), a complement to the third probe sequence (2′), a cleavable complement of the second internal cleavable site, and a complement to the second primer binding sequence (PB2′); and a second extended oligonucleotide conjugated to the second proximity probe including, from 5′ to 3′, a second primer binding sequence (PB2), a second internal cleavable site, a third probe sequence (3), a second barcode sequence (UMI2), a second probe sequence (2), a complement of the first barcode sequence (UMI1′), and a complement of first primer binding sequence complement (PB1′). The second internal cleavable site of the second probe oligonucleotide and the cleavable complement of the second internal cleavable site are then cleaved (e.g., by endonuclease digestion with an enzyme that recognizes the duplexed second cleavable site and cleavable complement of the second cleavable site, as illustrated by the lightning bolts), releasing the second extended oligonucleotide from the second proximity probe. FIG. 6C illustrates the steps of removing the cleaved second probe oligonucleotide (e.g., by lambda exonuclease digestion at the free 5′-PO4 of the second probe oligonucleotide), and subsequently hybridizing the first probe oligonucleotide to the third probe oligonucleotide on Protein B, wherein the complement of the third probe sequence (3′) of the first probe oligonucleotide anneals to the fourth probe sequence (4) of the third probe oligonucleotide.

FIG. 6D illustrates extension of the annealed Protein A and Protein B probe oligonucleotides. Using a polymerase, the 3′ end of each hybridized probe oligonucleotide is extended generating: a third extended oligonucleotide including, from 5′ to 3′, the first primer binding sequence (PB1), the first barcode sequence (UMI1), the first probe sequence (1), the complement of the second barcode sequence (UMI2′), the complement of the third probe sequence (3′), a complement of the third barcode sequence (UMI3′), a complement of the fifth probe sequence (5′), a complement of the second internal cleavable site, and the complement of the second primer binding sequence (PB2′); and a fourth extended oligonucleotide including, from 5′ to 3′, a second PLP binding sequence (PB2), a second internal cleavable site, a fifth probe sequence (5), a third barcode sequence (UMI3), the fourth probe sequence (4), the second barcode sequence (UMI2), the complement of the first probe sequence (1′), a complement of the first barcode sequence (UMI1′), and a complement of the first primer binding sequence (PB1′). The first cleavable site on the fourth extended oligonucleotide is then cleaved (e.g., RNAse cleavage of a ribonucleotide), releasing the fourth extended oligonucleotide from the antibody. In embodiments, the first cleavable site is located in the linker between the specific binding molecule (e.g., antibody) and the probe oligonucleotide, rather than at the 5′ end of the secondary probe oligonucleotide.

FIG. 6E illustrates the steps of removing the cleaved fourth extended oligonucleotide (e.g., by lambda exonuclease 5′ to 3′ digestion), and subsequently hybridizing a circularizable probe onto the third extended oligonucleotide, wherein the PB1′ region at the 5′ end of the probe anneals to the PB1 sequence of the third extended oligonucleotide, and wherein the PB2 region at the 3′ end anneals to the PB2′ sequence of the third extended oligonucleotide. FIG. 6F illustrates the steps of extending the 3′ end of the circularizable probe (e.g., using a non-strand displacing polymerase) to generate a complementary sequence, including from 3′ to 5′, the second internal cleavable site, a fifth probe sequence (5), a third barcode sequence (UMI3), the third probe sequence (3), the second barcode sequence (UMI2), the complement of the first barcode sequence (1′), and the complement of the first barcode sequence (UMI1′). Following extension, the 3′ end of the complementary sequence is ligated to the 5′ end of the circularizable probe using, for example, a ligase, thereby generating a circularized probe. The circularized probe may then be amplified and detected, for example by sequencing, as described in FIG. 3D.

The methods described herein provide a novel way to obtain a comprehensive in situ view of protein interactions without the need to perform ex situ sequencing or use laborious and expensive techniques such as mass spectrometry. Cellular protein interactomes are able to be identified in their native context without the need to introduce exogenously expressed proteins with affinity tags (e.g. FLAG and/or HA peptide epitopes). The barcoded proximity probes described herein can be scaled up or down to multiplex targeting of numerous protein complexes in a sample. These methods provide unique insight into the spatial localization of protein complexes, for example, how protein complex components may vary depending on the tissue or cell under investigation, or under disease conditions.

Although the Examples described supra and herein outline in situ sequencing approaches, it will be appreciated that the methods may be modified such that the barcode-containing oligonucleotides are removed from the cell (e.g., the cell is harvested and the oligonucleotides purified or captured using affinity capture) and then sequenced on an instrument ex situ. In embodiments, following extension of the first oligonucleotide to copy the barcode sequence of the second oligonucleotide, the double-stranded extended oligonucleotide is cleaved and removed from the cell. For example, the cleavable linker is cleaved, and the double-stranded oligonucleotide include the two or more barcode sequences is removed and sequenced outside of the cell using standard sequencing approaches (e.g., sequenced on a Singular Genomics G4™ system). Alternatively, the padlock probe including the complementary sequences of the two or more barcode sequences is purified and/or capture from the cell, and sequenced ex situ. The padlock probe may be circularized in the cell or after removal from the cell, and may be amplified prior to sequencing, wherein the amplification occurred in the cell or the amplification is performed outside of the cell prior to sequencing.

### Example 4: Characterizing Protein-Protein Interactions in Disease States

Protein interaction networks (e.g., network maps that annotate protein interactions with single or multiple binding partners in a given biological context) are useful resources in the abstraction of basic science knowledge and in the development of biomedical applications. By studying protein interaction networks, we can learn about the evolution of individual proteins and about the different systems in which they are involved. Due to their central role in biological function, protein interactions also control the mechanisms leading to healthy and diseased states in organisms. Diseases are often caused by mutations affecting the binding interface or leading to biochemically dysfunctional allosteric changes in proteins. Therefore, protein interaction networks can elucidate the molecular basis of disease, which in turn can inform methods for prevention, diagnosis, and treatment (see, Gonzalez M W and Kann M G. PLoS Comput. Biol. 2012; 8(12):e1002819). As protein interactions mediate the healthy states in all biological processes, it follows that they should be key targets of the molecular-based studies of biological diseased states.

Protein interactions are known to be disrupted or altered in several human disease states. For example, pathogen-host interactions play a key role in bacterial and viral infections. The Human papillomavirus, upon infection, expression two viral genes, E6 and E7, which interaction with negative cell regulatory proteins to target them for degradation, allowing the virus to bypass the immune system (see, Scheffner M et al. Semin. Cancer Biol. 2003; 13:59-67). In other diseases, such as Huntington's disease, cystic fibrois, and Alzheimer's disease, mutations may lead to unwanted protein interactions (e.g., mutations that lead to toxic misfolded proteins) that can alter homeostatic protein networks and lead to disease. In the case of Huntington's disease, glutamine expansion in the Huntingtin protein leads to alternate conformational states that induce toxic protein interactions (see, Duennwald M L et al. Proc. Natl. Acad. Sci. USA. 2006; 103(29): 11051-6). Further, oncogenic protein-protein interactions have been found to have a high correlation with patient survival and drug resistance/sensitivity, with the recent observation that somatic missense mutations are enriched at the interfaces of protein-protein interactions in cancer (see, Cheng F et al. Nat. Genet. 2021; 53:342-353, which in incorporated herein by reference in its entirety).

Amino acid substitutions in vinculin (VCL), located at the interaction interface between VCL and fragile X mental retardation syndrome-related protein 1 (FXFR1) are significantly correlated with resistance to incorafenib, an FDA-approved BRAF inhibitor for the treatment of melanoma, compared to patients without VCL-FXFR1-perturbing mutations (see, Koelblinger P et al. Curr. Opin. Oncol. 2018; 30:125-133). The methods described herein provide a novel in situ proteomic approach for obtaining detailed protein-protein interaction information from diseased tissue, such as tumor tissue, for example, in a patient undergoing treatment for melanoma. Briefly, a tumor tissue section is attached to a substrate surface, fixed, and permeabilized according to known methods in the art. The methods described in Example 2 are then performed, using a first proximal probe specific for VCL and a second proximal probe for FXFR1. Following extension and removal (e.g., digestion) of the second probe oligonucleotide, a circularizable probe is hybridized to the first probe oligonucleotide, extended, circularized, and amplified, as illustrated in FIGS. 3B-3D. This extension product is then primed with a sequencing primer and subjected to sequencing processes as described herein, thereby providing a high-resolution view of molecular features that can be combined with additional histological findings for clinical decision-making.

### P-EMBODIMENTS

Embodiment P1. A method of forming an oligonucleotide comprising two barcode sequences, said method comprising: a) contacting a first biomolecule with a first proximity probe, wherein the first proximity probe comprises a first oligonucleotide comprising, from 5′ to 3′, a first primer binding sequence, a first barcode sequence, and a first probe sequence; b) contacting a second biomolecule with a second proximity probe, wherein the second proximity probe comprises a second oligonucleotide comprising, from 5′ to 3′, a second primer binding sequence, a second barcode sequence, and a second probe sequence; c) hybridizing the first probe sequence of said first oligonucleotide to the second probe sequence of said second oligonucleotide and extending the first probe sequence with a polymerase to form a first extended oligonucleotide comprising, from 5′ to 3′, the first primer binding sequence, the first barcode sequence, the first probe sequence, a complement of the second barcode sequence, and a complement of the second primer binding sequence.

Embodiment P2. The method of Embodiment P1, wherein both the first and the second oligonucleotide comprise a first cleavable site.

Embodiment P3. The method of Embodiment P2, wherein the first cleavable site of the first oligonucleotide is 5′ of the first primer binding sequence, and wherein the first cleavable site of the second oligonucleotide is 5′ of the second primer binding sequence.

Embodiment P4. The method of Embodiment P1, wherein the second oligonucleotide comprises a first cleavable site.

Embodiment P5. The method of Embodiment P4, wherein the first cleavable site of the second oligonucleotide is 5′ of the second primer binding sequence.

Embodiment P6. The method of Embodiment P2 or Embodiment P3, comprising cleaving the first cleavable site, amplifying the first extended oligonucleotide comprising said two barcode sequences, or complements thereof, to form amplification products, and sequencing the amplification products.

Embodiment P7. The method of Embodiment P4 or Embodiment P5, comprising cleaving the first cleavable site and removing the second oligonucleotide.

Embodiment P8. The method of any one of Embodiment P1 to Embodiment P7, further comprising detecting the first extended oligonucleotide.

Embodiment P9. The method of Embodiment P7, further comprising hybridizing an oligonucleotide primer to the first extended oligonucleotide, wherein the oligonucleotide primer comprises, from 5′ to 3′, a first sequence complementary to the first primer binding sequence and a second sequence complementary to the complement of the second primer binding sequence, extending the second sequence along the first extended oligonucleotide to generate a complementary sequence, and ligating the complementary sequence to the first sequence of the oligonucleotide primer to form a circular oligonucleotide comprising the complement of the first barcode sequence and the second barcode sequence.

Embodiment P10. The method of Embodiment P1, wherein: the second oligonucleotide comprises, from 5′ to 3′, a second primer binding sequence, a second internal cleavable site, a third probe sequence, a second barcode sequence, and a second probe sequence, and the first extended oligonucleotide comprises, from 5′ to 3′, the first primer binding sequence, the first barcode sequence, the first probe sequence, a complement of the second barcode sequence, a complement of the third probe sequence, a cleavable complement of the second internal cleavable site, and a complement of the second primer binding sequence.

Embodiment P11. The method of Embodiment P10, further comprising: d) cleaving the second internal cleavable site of said second oligonucleotide and the cleavable complement of the second internal cleavable site of said first extended oligonucleotide, thereby forming a cleaved second oligonucleotide and a cleaved first extended oligonucleotide, and removing said cleaved second oligonucleotide.

Embodiment P12. The method of Embodiment P10, further comprising: d) extending the second oligonucleotide with a polymerase to form a second extended oligonucleotide comprising, from 5′ to 3′, the second primer binding sequence, the second internal cleavable site, the third probe sequence, the second barcode sequence, the second probe sequence, a complement of the first barcode sequence, and the second primer binding sequence.

Embodiment P13. The method of Embodiment P12, further comprising cleaving the second internal cleavable site of said second extended oligonucleotide and the cleavable complement of the second internal cleavable site of said first extended oligonucleotide, thereby forming a cleaved second extended oligonucleotide and a cleaved first extended oligonucleotide, and removing said cleaved second extended oligonucleotide.

Embodiment P14. The method of Embodiment P11 or Embodiment P13, wherein the cleaved first extended oligonucleotide comprises, from 5′ to 3′, the first primer binding sequence, the first barcode sequence, the first probe sequence, a complement of the second barcode sequence, and the complement of the third probe sequence.

Embodiment P15. The method of any one of Embodiment P11, Embodiment P13, or Embodiment P14, further comprising: e) contacting a third biomolecule with a third proximity probe, wherein the third proximity probe comprises a third oligonucleotide comprising, from 5′ to 3′, the second primer binding sequence, the second internal cleavable site, a fifth probe sequence, a third barcode sequence, and a fourth probe sequence; and f) hybridizing the complement of the third probe sequence of said cleaved first extended oligonucleotide to the fourth probe sequence of said third oligonucleotide and extending the complement of the third probe sequence with a polymerase to form a third extended oligonucleotide comprising, from 5′ to 3′, the first primer binding sequence, the first barcode sequence, the first probe sequence, the complement of the second barcode sequence, the complement of the third probe sequence, a complement of the third barcode sequence, a complement of the fifth probe sequence; the cleavable complement of the second internal cleavable site, and the complement of the second primer binding sequence.

Embodiment P16. The method of Embodiment P15, further comprising: g) extending the third oligonucleotide with the polymerase to form a fourth extended oligonucleotide comprising, from 5′ to 3′, the second primer binding sequence, the second internal cleavable site, the fifth probe sequence, the third barcode sequence, the fourth probe sequence, a complement of the first barcode sequence, a complement of the first probe sequence, the complement of the first barcode sequence, and the complement of the first primer binding sequence.

Embodiment P17. The method of Embodiment P15 or Embodiment P16, wherein the third oligonucleotide comprises the first cleavable site at or near the 5′ end.

Embodiment P18. The method of Embodiment P17, wherein the first cleavable site of the third oligonucleotide is 5′ of the second primer binding sequence.

Embodiment P19. The method of Embodiment P17 or Embodiment P18, comprising cleaving the first cleavable site of the third oligonucleotide, amplifying the third extended oligonucleotide comprising said three barcode sequences, or complements thereof, to form amplification products, and sequencing the amplification products.

Embodiment P20. The method of any one of Embodiment P15 to Embodiment P19, further comprising detecting the third extended oligonucleotide.

Embodiment P21. The method of Embodiment P17 or Embodiment P18, further comprising cleaving the first cleavable site at or near the 5′ end of the third oligonucleotide and removing the third oligonucleotide.

Embodiment P22. The method of Embodiment P17 or Embodiment P18, further comprising cleaving the first cleavable site at or near the 5′ end of the third oligonucleotide, removing the fourth extended oligonucleotide, and detecting the third extended oligonucleotide.

Embodiment P23. The method of Embodiment P21 or Embodiment P22, further comprising hybridizing an oligonucleotide primer to the third extended oligonucleotide, wherein the oligonucleotide primer comprises, from 5′ to 3′, a first sequence complementary to the first primer binding sequence and a second sequence complementary to the complement of the second primer binding sequence, extending the second sequence along the third extended oligonucleotide to generate a complementary sequence, and ligating the complementary sequence to the first sequence of the oligonucleotide primer to form a circular oligonucleotide comprising the complement of the first barcode sequence, the second barcode sequence, and the third barcode sequence.

Embodiment P24. The method of Embodiment P9 or Embodiment P23, further comprising amplifying the circular oligonucleotide by extending an amplification primer hybridized to the circular oligonucleotide with a strand-displacing polymerase, wherein the amplification primer extension generates an extension product comprising multiple complements of the circular oligonucleotide.

Embodiment P25. The method of Embodiment P9 or Embodiment P23, further comprising sequencing the circular oligonucleotide.

Embodiment P26. The method of Embodiment P24, further comprising sequencing the extension product.

Embodiment P27. The method of any one of Embodiment P1 to Embodiment P26, wherein said first oligonucleotide is attached to the first proximity probe via a linker, and wherein said second oligonucleotide is attached to the second proximity probe via a linker.

Embodiment P28. The method of Embodiment P27, wherein said second oligonucleotide is attached to the second proximity probe via a cleavable linker.

Embodiment P29. The method of any one of Embodiment P15 to Embodiment P26, wherein said third oligonucleotide is attached to the third proximity probe via a cleavable linker.

Embodiment P30. The method of Embodiment P28 or Embodiment P29, wherein said cleavable linker comprises a polynucleotide or a polypeptide sequence.

Embodiment P31. The method of any one of Embodiment P1 to Embodiment P30, wherein the proximity probe is an antibody, an antibody fragment, an affimer, an aptamer, or a nucleic acid.

Embodiment P32. A composition comprising: i) a biomolecule bound to a proximity probe, wherein the proximity probe comprises an extended probe oligonucleotide comprising, from 5′ to 3′, a first primer binding sequence, a first barcode sequence, a first probe sequence, a complement of a second barcode sequence, and a complement of a second primer binding sequence; and ii) an oligonucleotide primer hybridized to the extended probe oligonucleotide, wherein the oligonucleotide primer comprises, from 5′ to 3′, a first sequence complementary to the first primer binding sequence and a second sequence complementary to the complement of the second primer binding sequence.

Embodiment P33. A composition comprising: i) a biomolecule bound by a proximity probe, wherein the proximity probe comprises an extended probe oligonucleotide comprising, from 5′ to 3′, a first primer binding sequence, a first barcode sequence, a first probe sequence, a complement of a second barcode sequence, a complement of a third probe sequence, a complement of a third barcode sequence, a complement of a fifth probe sequence, an internal cleavable site, and a complement of a second primer binding sequence; and ii) an oligonucleotide primer hybridized to the extended probe oligonucleotide, wherein the oligonucleotide primer comprises, from 5′ to 3′, a first sequence complementary to the first primer binding sequence and a second sequence complementary to the complement of the second primer binding sequence.

What is claimed is:

1. A method of forming an oligonucleotide comprising two barcode sequences, said method comprising:
a) contacting a first biomolecule with a first proximity probe, wherein the first proximity probe comprises a first oligonucleotide comprising, from 5′ to 3′, a first primer binding sequence, a first barcode sequence, and a first probe sequence;
b) contacting a second biomolecule with a second proximity probe, wherein the second proximity probe comprises a second oligonucleotide comprising, from 5′ to 3′, a second primer binding sequence, a second barcode sequence, and a second probe sequence;
c) hybridizing the first probe sequence of said first oligonucleotide to the second probe sequence of said second oligonucleotide and extending the first probe sequence with a polymerase to form a first extended oligonucleotide comprising, from 5′ to 3′, the first primer binding sequence, the first barcode sequence, the first probe sequence, a complement of the second barcode sequence, and a complement of the second primer binding sequence.

2. The method of claim 1, wherein the first oligonucleotide and the second oligonucleotide comprise a first cleavable site.

3. The method of claim 2, wherein the first cleavable site of the first oligonucleotide is 5′ of the first primer binding sequence, and wherein the first cleavable site of the second oligonucleotide is 5′ of the second primer binding sequence.

4. The method of claim 1, wherein the second oligonucleotide comprises a first cleavable site.

5. The method of claim 2, further comprising cleaving the first cleavable site, amplifying the first extended oligonucleotide to form amplification products, and sequencing the amplification products.

6. The method of claim 4, further comprising cleaving the first cleavable site and removing the second oligonucleotide.

7. The method of claim 6, further comprising hybridizing an oligonucleotide primer to the first extended oligonucleotide, wherein the oligonucleotide primer comprises, from 5′ to 3′, a first sequence complementary to the first primer binding sequence and a second sequence complementary to the complement of the second primer binding sequence, extending the second sequence along the first extended oligonucleotide to generate a complementary sequence, and ligating the complementary sequence to the first sequence of the oligonucleotide primer to form a circular oligonucleotide comprising the complement of the first barcode sequence and the second barcode sequence.

5. The method of claim 1, wherein:
the second oligonucleotide comprises, from 5′ to 3′, a second primer binding sequence, a second internal cleavable site, a third probe sequence, a second barcode sequence, and a second probe sequence, and
the first extended oligonucleotide comprises, from 5′ to 3′, the first primer binding sequence, the first barcode sequence, the first probe sequence, a complement of the second barcode sequence, a complement of the third probe sequence, a cleavable complement of the second internal cleavable site, and a complement of the second primer binding sequence.

9. The method of claim 8, further comprising:
d) cleaving the second internal cleavable site of said second oligonucleotide and the cleavable complement of the second internal cleavable site of said first extended oligonucleotide, thereby forming a cleaved second oligonucleotide and a cleaved first extended oligonucleotide, and removing said cleaved second oligonucleotide.

10. The method of claim 8, further comprising:
d) extending the second oligonucleotide with a polymerase to form a second extended oligonucleotide comprising, from 5′ to 3′, the second primer binding sequence, the second internal cleavable site, the third probe sequence, the second barcode sequence, the second probe sequence, a complement of the first barcode sequence, and the second primer binding sequence.

11. The method of claim 10, further comprising cleaving the second internal cleavable site of said second extended oligonucleotide and the cleavable complement of the second internal cleavable site of said first extended oligonucleotide, thereby forming a cleaved second extended oligonucleotide and a cleaved first extended oligonucleotide, and removing said cleaved second extended oligonucleotide.

12. The method of claim 9, wherein the cleaved first extended oligonucleotide comprises, from 5′ to 3′, the first primer binding sequence, the first barcode sequence, the first probe sequence, a complement of the second barcode sequence, and the complement of the third probe sequence.

13. The method of claim 7, further comprising amplifying the circular oligonucleotide by extending an amplification primer hybridized to the circular oligonucleotide with a strand-displacing polymerase, wherein the amplification primer extension generates an extension product comprising multiple complements of the circular oligonucleotide.

14. The method of claim 7, further comprising sequencing the circular oligonucleotide.

15. The method of claim 13, further comprising sequencing the extension product.

6. The method of claim 1, wherein said first oligonucleotide is attached to the first proximity probe via a linker, and wherein said second oligonucleotide is attached to the second proximity probe via a cleavable linker.

7. The method of claim 16, wherein said cleavable linker comprises a polynucleotide or a polypeptide sequence.

8. The method of claim 1, wherein the first proximity probe and the second proximity probe are an antibody, an antibody fragment, an affimer, an aptamer, or a nucleic acid.

9. A composition comprising:
i) a biomolecule bound to a proximity probe, wherein the proximity probe comprises an extended probe oligonucleotide comprising, from 5′ to 3′, a first primer binding sequence, a first barcode sequence, a first probe sequence, a complement of a second barcode sequence, and a complement of a second primer binding sequence; and
ii) an oligonucleotide primer hybridized to the extended probe oligonucleotide, wherein the oligonucleotide primer comprises, from 5′ to 3′, a first sequence complementary to the first primer binding sequence and a second sequence complementary to the complement of the second primer binding sequence.

10. A composition comprising:
i) a biomolecule bound by a proximity probe, wherein the proximity probe comprises an extended probe oligonucleotide comprising, from 5′ to 3′, a first primer binding sequence, a first barcode sequence, a first probe sequence, a complement of a second barcode sequence, a complement of a third probe sequence, a complement of a third barcode sequence, a complement of a fifth probe sequence, an internal cleavable site, and a complement of a second primer binding sequence; and
ii) an oligonucleotide primer hybridized to the extended probe oligonucleotide, wherein the oligonucleotide primer comprises, from 5′ to 3′, a first sequence complementary to the first primer binding sequence and a second sequence complementary to the complement of the second primer binding sequence.


SEQUENCE LISTING (74 sequences)

SEQ ID NO: 1-20 - skipped (20 entries)

SEQ ID NO: 21 - DNA, 29 bp, synthetic construct
  misc_feature 1..29: note = Synthetic construct
  source 1..29: mol_type = other DNA
acaaaggcagccacgcactccttccctgt

SEQ ID NO: 22 - DNA, 33 bp, synthetic construct
  misc_feature 1..33: note = Synthetic construct
  source 1..33: mol_type = other DNA
acactctttccctacacgacgctcttccgatct

SEQ ID NO: 23 - DNA, 29 bp, synthetic construct
  misc_feature 1..29: note = Synthetic construct
  source 1..29: mol_type = other DNA
ctccagcgagatgaccctcaccaaccact

SEQ ID NO: 24 - DNA, 34 bp, synthetic construct
  misc_feature 1..34: note = Synthetic construct
  source 1..34: mol_type = other DNA
gtgactggagttcagacgtgtgctcttccgatct

SEQ ID NO: 25 - DNA, 19 bp, synthetic construct
  misc_feature 1..19: note = Synthetic construct
  source 1..19: mol_type = other DNA
aatgatacggcgaccaccg

SEQ ID NO: 26 - DNA, 24 bp, synthetic construct
  misc_feature 1..24: note = Synthetic construct
  source 1..24: mol_type = other DNA
caagcagaagacggcatacgagat

SEQ ID NO: 27 - DNA, 33 bp, synthetic construct
  misc_feature 1..33: note = Synthetic construct
  source 1..33: mol_type = other DNA
aacgccaaacctacggctttacttcctgtggct

SEQ ID NO: 28 - DNA, 33 bp, synthetic construct
  misc_feature 1..33: note = Synthetic construct
  source 1..33: mol_type = other DNA
tcttgagtcattcgcagggcatgtgccagacct

SEQ ID NO: 29 - DNA, 33 bp, synthetic construct
  misc_feature 1..33: note = Synthetic construct
  source 1..33: mol_type = other DNA
tcggcgttgtctgctatcgttcttggcactcct

SEQ ID NO: 30 - DNA, 33 bp, synthetic construct
  misc_feature 1..33: note = synthetic construct
  source 1..33: mol_type = other DNA
ggagcaataaccataaggccgttgacaagccct

SEQ ID NO: 31 - DNA, 33 bp, synthetic construct
  misc_feature 1..33: note = synthetic construct
  source 1..33: mol_type = other DNA
ggcgtattgccttggttctggcagcctcattgt

SEQ ID NO: 32 - DNA, 33 bp, synthetic construct
  misc_feature 1..33: note = synthetic construct
  source 1..33: mol_type = other DNA
cagcagagggaacgatttcaacttcctgtggct

SEQ ID NO: 33 - DNA, 33 bp, synthetic construct
  misc_feature 1..33: note = Synthetic construct
  source 1..33: mol_type = other DNA
ctactgcaagggtgtctagaatgtgccagacct

SEQ ID NO: 34 - DNA, 33 bp, synthetic construct
  misc_feature 1..33: note = synthetic construct
  source 1..33: mol_type = other DNA
gaccgactcgtgaaacgtaatcttggcactcct

SEQ ID NO: 35 - DNA, 33 bp, synthetic construct
  misc_feature 1..33: note = synthetic construct
  source 1..33: mol_type = other DNA
acacattctttgcgcccagagttgacaagccct

SEQ ID NO: 36 - DNA, 33 bp, synthetic construct
  misc_feature 1..33: note = synthetic construct
  source 1..33: mol_type = other DNA
atttcattcgacacccggtcgcagcctcattgt

SEQ ID NO: 37 - DNA, 33 bp, synthetic construct
  misc_feature 1..33: note = synthetic construct
  source 1..33: mol_type = other DNA
agccacaggaagtaaagccgtaggtttggcgtt

SEQ ID NO: 38 - DNA, 33 bp, synthetic construct
  misc_feature 1..33: note = synthetic construct
  source 1..33: mol_type = other DNA
aggtctggcacatgccctgcgaatgactcaaga

SEQ ID NO: 39 - DNA, 33 bp, synthetic construct
  misc_feature 1..33: note = Synthetic construct
  source 1..33: mol_type = other DNA
aggagtgccaagaacgatagcagacaacgccga

SEQ ID NO: 40 - DNA, 33 bp, synthetic construct
  misc_feature 1..33: note = synthetic construct
  source 1..33: mol_type = other DNA
agggcttgtcaacggccttatggttattgctcc

SEQ ID NO: 41 - DNA, 33 bp, synthetic construct
  misc_feature 1..33: note = synthetic construct
  source 1..33: mol_type = other DNA
acaatgaggctgccagaaccaaggcaatacgcc

SEQ ID NO: 42 - DNA, 33 bp, synthetic construct
  misc_feature 1..33: note = synthetic construct
  source 1..33: mol_type = other DNA
agccacaggaagttgaaatcgttccctctgctg

SEQ ID NO: 43 - DNA, 33 bp, synthetic construct
  misc_feature 1..33: note = synthetic construct
  source 1..33: mol_type = other DNA
aggtctggcacattctagacacccttgcagtag

SEQ ID NO: 44 - DNA, 33 bp, synthetic construct
  misc_feature 1..33: note = synthetic construct
  source 1..33: mol_type = other DNA
aggagtgccaagattacgtttcacgagtcggtc

SEQ ID NO: 45 - DNA, 33 bp, synthetic construct
  misc_feature 1..33: note = synthetic construct
  source 1..33: mol_type = other DNA
agggcttgtcaactctgggcgcaaagaatgtgt

SEQ ID NO: 46 - DNA, 33 bp, synthetic construct
  misc_feature 1..33: note = synthetic construct
  source 1..33: mol_type = other DNA
acaatgaggctgcgaccgggtgtcgaatgaaat

SEQ ID NO: 47 - DNA, 33 bp, synthetic construct
  misc_feature 1..33: note = synthetic construct
  source 1..33: mol_type = other DNA
tgttgcatctccacccggattgagccttcagct

SEQ ID NO: 48 - DNA, 33 bp, synthetic construct
  misc_feature 1..33: note = synthetic construct
  source 1..33: mol_type = other DNA
cacaacgggagctgtggaattggttcacctggt

SEQ ID NO: 49 - DNA, 33 bp, synthetic construct
  misc_feature 1..33: note = synthetic construct
  source 1..33: mol_type = other DNA
tggactaagactcgtcctccagcggacctaagt

SEQ ID NO: 50 - DNA, 33 bp, synthetic construct
  misc_feature 1..33: note = synthetic construct
  source 1..33: mol_type = other DNA
gtatgatggtgttgcggcttctcgcttaacgct

SEQ ID NO: 51 - DNA, 33 bp, synthetic construct
  misc_feature 1..33: note = synthetic construct
  source 1..33: mol_type = other DNA
tctgagtgccagtgacttcacgcattcgcttgt

SEQ ID NO: 52 - DNA, 33 bp, synthetic construct
  misc_feature 1..33: note = synthetic construct
  source 1..33: mol_type = other DNA
tacgacacactcgggctctatgggcttcatggt

SEQ ID NO: 53 - DNA, 33 bp, synthetic construct
  misc_feature 1..33: note = synthetic construct
  source 1..33: mol_type = other DNA
gtttgagtgaaggcggtccaacccttagtgcgt

SEQ ID NO: 54 - DNA, 33 bp, synthetic construct
  misc_feature 1..33: note = synthetic construct
  source 1..33: mol_type = other DNA
ctataagtttgtcgtgcccgtgagccttcagct

SEQ ID NO: 55 - DNA, 33 bp, synthetic construct
  misc_feature 1..33: note = synthetic construct
  source 1..33: mol_type = other DNA
ggagtgacactgactacgtttggttcacctggt

SEQ ID NO: 56 - DNA, 33 bp, synthetic construct
  misc_feature 1..33: note = synthetic construct
  source 1..33: mol_type = other DNA
gtcaacgccctagcagacatagcggacctaagt

SEQ ID NO: 57 - DNA, 33 bp, synthetic construct
  misc_feature 1..33: note = synthetic construct
  source 1..33: mol_type = other DNA
ccagaacctattgagcctgactcgcttaacgct

SEQ ID NO: 58 - DNA, 33 bp, synthetic construct
  misc_feature 1..33: note = synthetic construct
  source 1..33: mol_type = other DNA
aggtgttcgtacaatgaggccgcattcgcttgt

SEQ ID NO: 59 - DNA, 33 bp, synthetic construct
  misc_feature 1..33: note = synthetic construct
  source 1..33: mol_type = other DNA
tggtcaagggcaactaatcctgggcttcatggt

SEQ ID NO: 60 - DNA, 33 bp, synthetic construct
  misc_feature 1..33: note = synthetic construct
  source 1..33: mol_type = other DNA
acaattacccgtttaccggcacccttagtgcgt

SEQ ID NO: 61 - DNA, 33 bp, synthetic construct
  misc_feature 1..33: note = synthetic construct
  source 1..33: mol_type = other DNA
agctgaaggctcaatccgggtggagatgcaaca

SEQ ID NO: 62 - DNA, 33 bp, synthetic construct
  misc_feature 1..33: note = synthetic construct
  source 1..33: mol_type = other DNA
accaggtgaaccaattccacagctcccgttgtg

SEQ ID NO: 63 - DNA, 33 bp, synthetic construct
  misc_feature 1..33: note = synthetic construct
  source 1..33: mol_type = other DNA
acttaggtccgctggaggacgagtcttagtcca

SEQ ID NO: 64 - DNA, 33 bp, synthetic construct
  misc_feature 1..33: note = synthetic construct
  source 1..33: mol_type = other DNA
agcgttaagcgagaagccgcaacaccatcatac

SEQ ID NO: 65 - DNA, 33 bp, synthetic construct
  misc_feature 1..33: note = synthetic construct
  source 1..33: mol_type = other DNA
acaagcgaatgcgtgaagtcactggcactcaga

SEQ ID NO: 66 - DNA, 33 bp, synthetic construct
  misc_feature 1..33: note = synthetic construct
  source 1..33: mol_type = other DNA
accatgaagcccatagagcccgagtgtgtcgta

SEQ ID NO: 67 - DNA, 33 bp, synthetic construct
  misc_feature 1..33: note = synthetic construct
  source 1..33: mol_type = other DNA
acgcactaagggttggaccgccttcactcaaac

SEQ ID NO: 68 - DNA, 33 bp, synthetic construct
  misc_feature 1..33: note = synthetic construct
  source 1..33: mol_type = other DNA
agctgaaggctcacgggcacgacaaacttatag

SEQ ID NO: 69 - DNA, 33 bp, synthetic construct
  misc_feature 1..33: note = synthetic construct
  source 1..33: mol_type = other DNA
accaggtgaaccaaacgtagtcagtgtcactcc

SEQ ID NO: 70 - DNA, 33 bp, synthetic construct
  misc_feature 1..33: note = synthetic construct
  source 1..33: mol_type = other DNA
acttaggtccgctatgtctgctagggcgttgac

SEQ ID NO: 71 - DNA, 33 bp, synthetic construct
  misc_feature 1..33: note = synthetic construct
  source 1..33: mol_type = other DNA
agcgttaagcgagtcaggctcaataggttctgg

SEQ ID NO: 72 - DNA, 33 bp, synthetic construct
  misc_feature 1..33: note = synthetic construct
  source 1..33: mol_type = other DNA
acaagcgaatgcggcctcattgtacgaacacct

SEQ ID NO: 73 - DNA, 33 bp, synthetic construct
  misc_feature 1..33: note = synthetic construct
  source 1..33: mol_type = other DNA
accatgaagcccaggattagttgcccttgacca

SEQ ID NO: 74 - DNA, 33 bp, synthetic construct
  misc_feature 1..33: note = synthetic construct
  source 1..33: mol_type = other DNA
acgcactaagggtgccggtaaacgggtaattgt